(19) United States
(12) Patent Application Publication
(10) Pub. No.: US 2026/0152467 A1
(43) Pub. Date: Jun. 4, 2026

(54) DISPERSING AND WETTING AGENTS AND COMPOSITIONS AND METHODS OF USE THEREOF

(71) Applicant: MOJAVE BIO, INC., Los Altos, CA (US)

(72) Inventor: Eric STIMPSON, Montgomery, OH (US)

(21) Appl. No.: 19/114,750

(22) PCT Filed: Sep. 26, 2023
(86) PCT No.: PCT/US2023/033747

§ 371 Date: Mar. 24, 2025

Related U.S. Application Data
(60) Provisional application No. 63/410,186, filed on Sep. 26, 2022.

(51) Int. Cl. C07C 233/69; C08F 132/06; C08F 134/00; G01N 33/53; G01N 33/533

(52) U.S. Cl. CPC C07C 233/69; C08F 132/06; C08F 134/00; G01N 33/5306; G01N 33/533

(57) ABSTRACT

Aspects of the disclosure include compositions, reaction mixtures, and methods of use thereof, for preventing or reversing aggregation of molecular entities prone to aggregation.

### CROSS-REFERENCE TO RELATED APPLICATIONS

This application claims priority to, and the benefit of, U.S. Provisional Patent Application No. 63/410,186, filed Sep. 26, 2022, the entire contents of which are herein incorporated by reference in its entirety for all purposes.

### FIELD OF DISCLOSURE

The present disclosure relates generally to fluorophores and use thereof, and particularly to compounds, compositions, and methods of their use that prevent or significantly reduce fluorophore aggregation.

### BACKGROUND OF THE DISCLOSURE

Fluorophores are microscopic molecules, including but not limited to proteins, small organic or inorganic compounds, and synthetic polymers that absorb light of specific wavelengths and emit light of longer wavelengths. Use of fluorophores has revolutionized the ability to probe biological phenomenon, including the monitoring of proteins, RNA, DNA, small molecules, cells, and even cellular properties such as pH and membrane potential. Accordingly, fluorescent dyes find use in a variety of applications in biochemistry, biology, and medicine, e.g., in diagnostic kits, in microscopy applications, fluorescence activated cell sorting (FACS), drug screening applications, and the like.

However, there are a myriad of technical issues that can limit the effectiveness of the use of fluorophores to probe biological phenomenon. Examples include intrinsic brightness, degree to which intrinsic background of a sample overlaps with the dye's emission, broadness of excitation and emission spectra that can lead to overlap of multiple probe emissions, resistance to photobleaching, and the like. Accordingly, efforts are continually underway to improve fluorophore capability with regard to one or more of the aforementioned issues, with varying levels of success.

Under conditions where a particular use-case relies on a plurality (e.g., two or more) of different fluorophores, dye aggregation represents yet another technical issue that can limit the effectiveness of the use of fluorophores to probe biological phenomenon. As a representative example, dye aggregation in FACS analysis can result in impure spectral signatures and distorted maximum fluorescence intensities (MFIs), hence degrading the ability the effectively interpret experimental results. Dye aggregation artifacts can thus adversely impact the use of fluorophores, even under circumstances where in theory the selected dyes do not exhibit overlaps in emissions or exhibit any otherwise undesirable properties. Dye aggregation artifacts are not solely relevant to use-cases where a plurality of dyes are used, as even in single dye use-cases, chemical properties of the dye can lead to reduced yield, e.g., where the selected dye has an affinity for the labware used in the particular application. Accordingly, the issue of dye aggregation/affinity artifacts represents a clear technical problem in need of a technical solution.

### SUMMARY OF DISCLOSURE

The present disclosure provides compounds, compositions, and reaction mixtures thereof that substantially reduce or eliminate aggregation issues for fluorophores that exhibit, in particular, hydrophobic interaction characteristics. Additionally provided are methods of use of said compounds, compositions, and reaction mixtures. The compounds, compositions, reaction mixtures, and methods of use thereof disclosed herein are particularly advantageous with regard to applications that rely on a plurality of different fluorophores (e.g., multiplex fluorescence assays, FACS, and the like).

In one aspect the disclosure provides a polymer comprising a saturated hydrocarbon substituted with at least one or more substituents, wherein each substituent, individually in each instance is (a) selected from the group consisting of aryl, alkyl-aryl, heteroalkyl-aryl; heteroaryl, alkyl-heteroaryl, heteroalkyl-heteroaryl, and combinations thereof; and (b) non-ionic at 6≤pH≤8, wherein the polymer (c) is water soluble, and (d) is transparent to 350 nm to 800 nm wavelength light. In some examples, the substituents selected from the group consisting of aryl, alkyl-aryl; heteroalkyl-aryl; heteroaryl, alkyl-heteroaryl, heteroalkyl-heteroaryl, and combinations thereof, are further substituted with one to six substituents selected from the group consisting of polyethylene glycol (PEG), —OH, —SH, —NO₂, —NO₃, —C(O)OH, —NH₃, —CH₃, —CH₂—OH, —CH₂—CH₂—OH, and —CN, wherein the optional substituents are not further substituted.

In some examples, the polymer does not exhibit an aqueous critical micelle concentration (CMC_{aq}).

In a first embodiments, the at least one or more substituents constitute 5%-20% of the polymer w/w.

In some examples, the at least one or more substituents constitute 1% to 100% of the substituents present on the polymer.

In some other examples, the at least one or more substituents constitute 1% to 50% of the substituents present on the polymer.

In some other examples, the at least one or more substituents constitute 1% to 20% of the substituents present on the polymer.

In some other examples, the at least one or more substituents constitute 5% to 20% of the substituents present on the polymer.

For certain applications, different amounts and/or types of substituents may be preferred.

In a second embodiment that optionally includes the first embodiment, the polymer is non-ionic at 6≤pH≤8.

In a third embodiment, optionally including any one or more or each of the first through second embodiments, the at least one or more substituents comprises two or more substituents, for example five or more substituents.

In a fourth embodiment, which optionally includes any one or more or each of the first through third embodiments, the at least one or more substituents are substituted with PEG linkers. In some examples, the PEG linkers comprise mPEG₆. In some examples, the PEG linkers comprise mPEG₅. In some examples, the PEG linkers comprise mPEG₄. In some examples, the PEG linkers comprise mPEG₃. In some examples, the PEG linkers comprise mPEG₂. In some examples, the PEG linkers comprise mPEG.

In a fifth embodiment, which optionally includes any one or more or each of the first through fourth embodiments, the polymer has an aqueous solubility of ≥1 mg/mL, for example an aqueous solubility of ≥10 mg/mL, or ≥100 mg/mL.

In a sixth embodiment, which optionally includes any one or more or each of the first through fifth embodiments, the polymer has average molecular weight (MW) between 500-5,000,000 Daltons, for example an average MW between 1000-100,000 Daltons, for example between 1000-10,000 Daltons.

In a seventh embodiment, which optionally includes any one or more or each of the first through sixth embodiments, the polymer comprises poly(vinyl alcohol). According to the seventh embodiment where the polymer comprises poly(vinyl alcohol), in some examples at least one or more substitutes are selected from alkyl-aryl. In a particular example the polymer is mPEG₄ and the alkyl-aryl is benzaldehyde.

In an eighth embodiment, which optionally includes any one or more or each of the first through seventh embodiments, the polymer is made by a process that comprises polymerizing monomers having an aqueous solubility of ≥10 mg/mL.

In a ninth embodiment, which optionally includes any one or more or each of the first through eighth embodiments, the polymer is made by a process that comprises radical or ionic polymerization of vinyl monomers.

In a tenth embodiment, which optionally incudes any one or more or each of the first through eighth embodiments, the polymer is made by a process that comprises post polymerization modification of polyvinyl alcohol (PVA).

In an eleventh embodiment, which optionally includes any one or more or each of the first through eighth embodiments, the polymer is made by a process that comprises arylation of PVA by using acetalization of the at least one or more substituents as monomers.

In a twelfth embodiment, which optionally includes any one or more or each of the first through eighth embodiments, the polymer is made by a process that comprises post polymerization modification of styrene-alt-maleic anhydride copolymer—SMA (50:50).

In a thirteenth embodiment, which optionally includes any one or more or each of the first through eighth embodiments, the polymer is made by a process that comprises aromatic monomer copolymerization with maleic anhydride and post polymerization modification of the maleic anhydride.

In a fourteenth embodiment, which optionally includes any one or more or each of the first through eighth embodiments, the polymer is made by a process that comprises post polymerization modification of methyl vinyl ether-alt-maleic anhydride copolymer (50:50).

In a fifteenth embodiment, which optionally includes any one or more or each of the first through ninth embodiments, wherein the polymer has a structure selected from Formula (I)

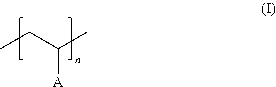

In examples according to the fifteenth embodiment, A is, independently in each instance, selected from the group consisting of selected from the group consisting of aryl, alkyl-aryl, heteroalkyl-aryl; heteroaryl, alkyl-heteroaryl, and heteroalkyl-heteroaryl, and subscript n is an integer from 1 to 100,000.

In some examples according to the fifteenth embodiment, A is selected from:

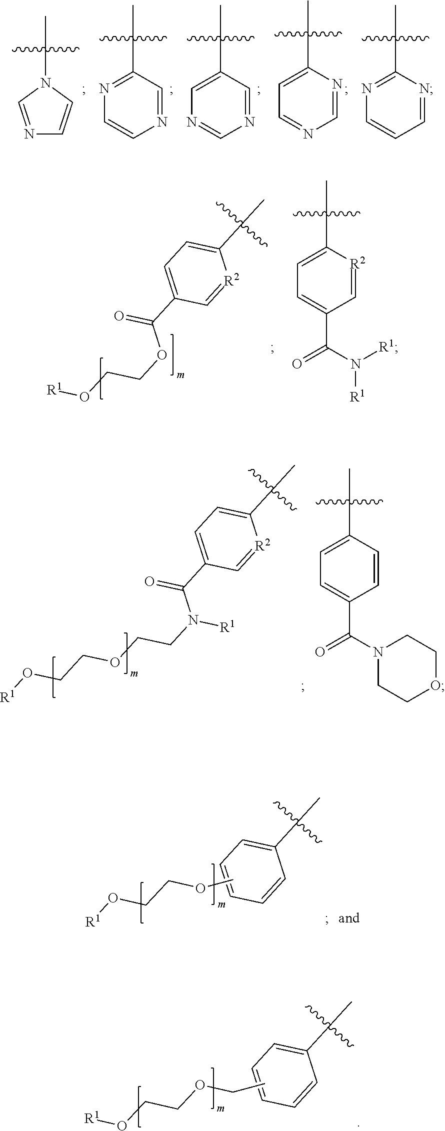

In such an example, subscript m is an integer from 1 to 10; R¹ is H or CH₃; R² is CH or N; and

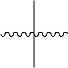

is the bond from A to Formula (I).

In a sixteenth embodiment, which optionally includes any one or more or each of the first through ninth embodiments, the polymer has a structure selected from Formula (II):

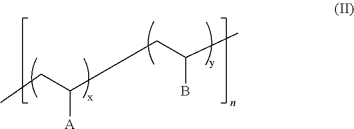

In examples according to the sixteenth embodiment, A and B are each, independently in each instance, selected from the group consisting of aryl, alkyl-aryl; heteroalkyl-aryl; heteroaryl, alkyl-heteroaryl, and heteroalkyl-heteroaryl. In examples, subscript x is an integer from 1 to 50, subscript y is an integer from 1 to 50, and subscript n is an integer from 1 to 100,000.

In examples according to the sixteenth embodiment, B is selected from the group consisting of:

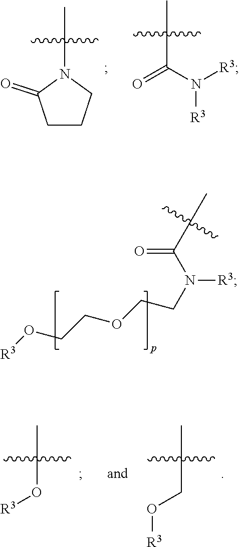

In such an example, subscript p is an integer from 1 to 10, wherein R³ is H or CH₃, and

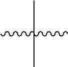

is the bond from B to Formula (II).

In examples according to the sixteenth embodiment, A is selected from:

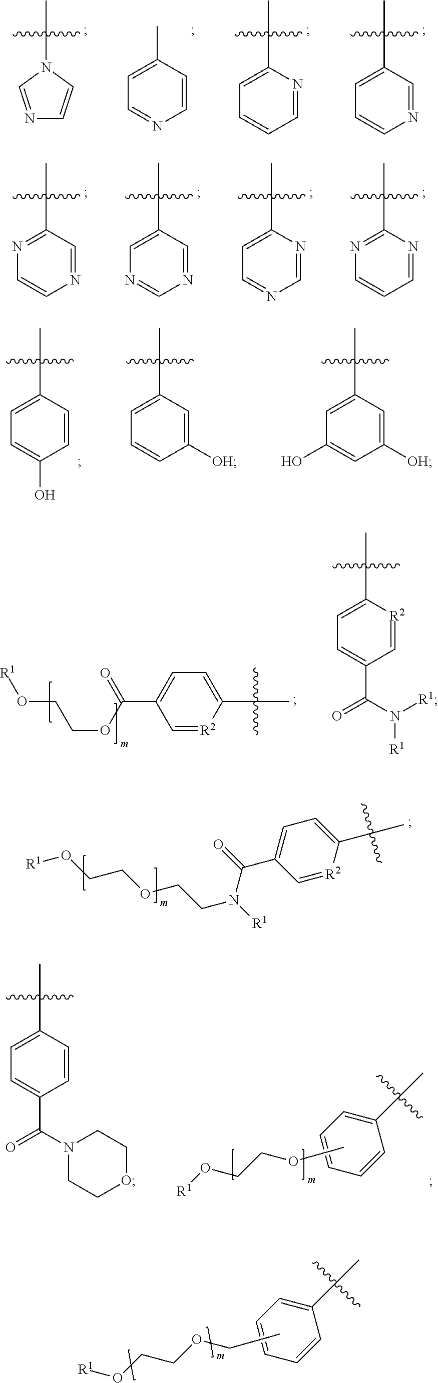

In such an example, subscript mi is an integer from 1 to 10, R¹ is H or CH₃, R² is CH or N, and

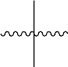

is the bond from A to Formula (11).

In a seventeenth embodiment, which optionally includes any one or more or each of the first through eighth embodiments and/or the tenth embodiment, the polymer has a structure selected from:

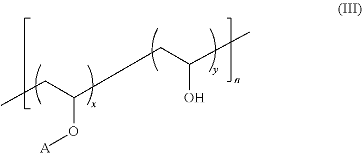

In examples according to the seventeenth embodiment, A is, independently in each instance, selected from the group consisting of aryl, alkyl-aryl; heteroalkyl-aryl; heteroaryl, alkyl-heteroaryl, and heteroalkyl-heteroaryl, subscript x is an integer from 1 to 50, subscript y is an integer from 1 to 50, and subscript n is an integer from 1 to 100,000.

In such an example, A is selected from:

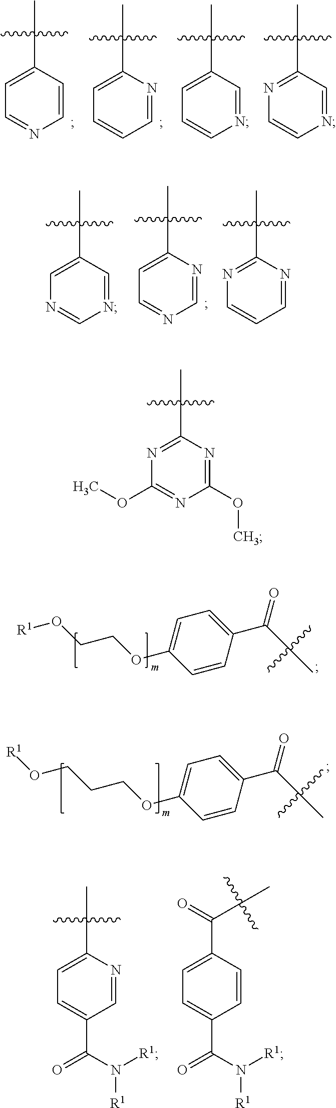

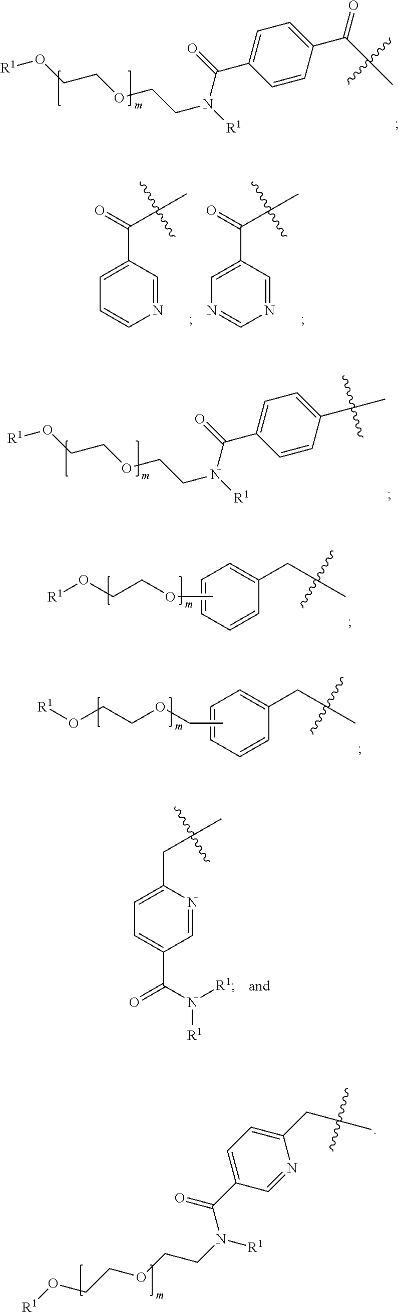

In such an example, subscript m is an integer from 1 to 10, R¹ is H or CH₃, R² is CH or N, and

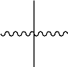

is the bond from A to Formula (III).

In an eighteenth embodiment, which optionally includes any one or more or each of the first through eighth embodiments and/or the eleventh embodiment, the polymer has a structure selected from Formula (IV):

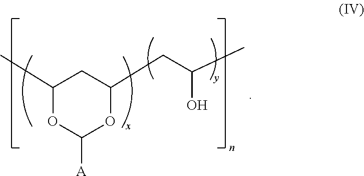

In examples according to the eighteenth embodiment, A is, independently in each instance, selected from the group consisting of aryl, alkyl-aryl; heteroalkyl-aryl; heteroaryl, alkyl-heteroaryl, and heteroalkyl-heteroaryl, subscript x is an integer from 1 to 50, subscript y is an integer from 1 to 50, and subscript n is an integer from 1 to 100,000.

In such an example, A is selected from:

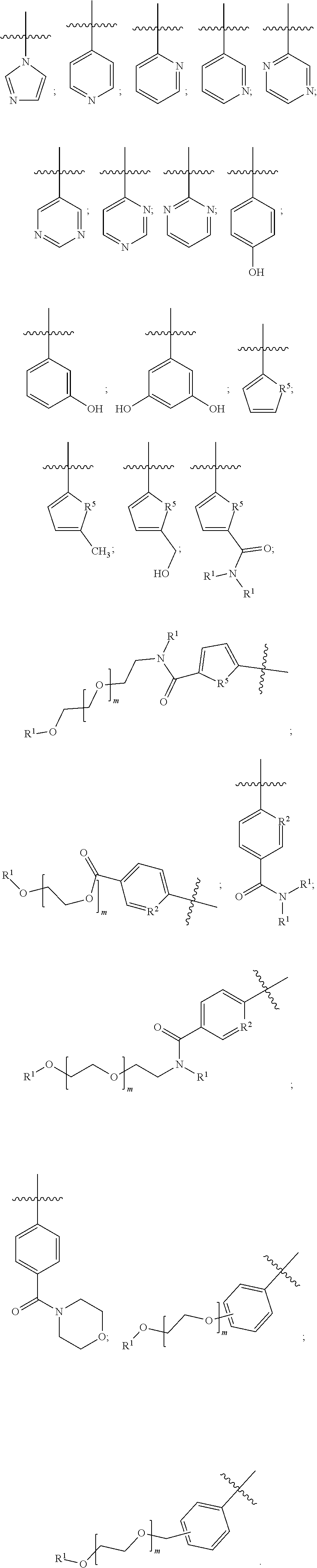

In such an example, subscript m is an integer from 1 to 10, R¹ is H or CH₃, R² is CH or N, R⁵ is O or S, and

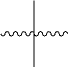

is the bond from A to Formula (III).

In a nineteenth embodiment, which optionally includes any one or more or each of the first through eighth embodiments and/or the twelfth embodiment, the polymer has a structure selected from Formula (V), (VI), (VII), or (VIII):

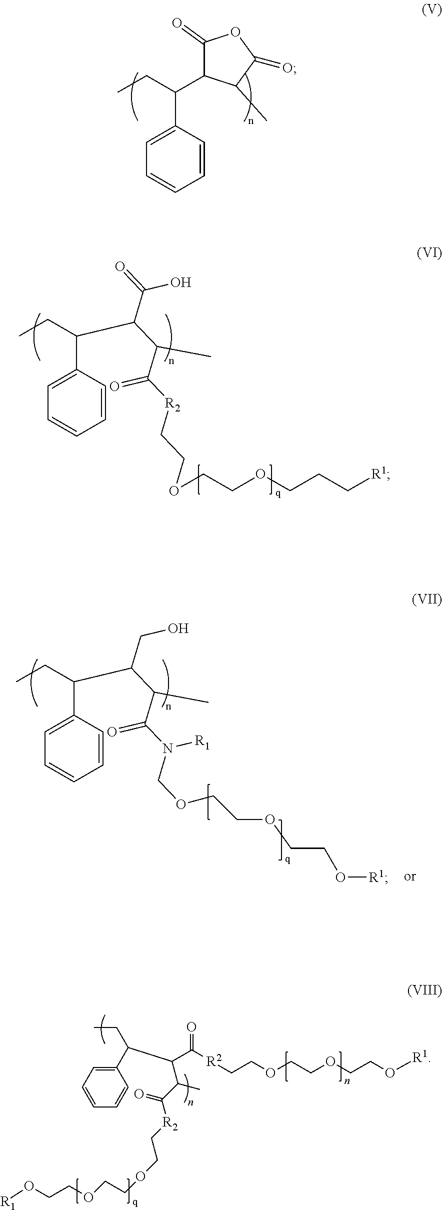

In examples according to the nineteenth embodiment, subscript n is an integer from 1 to 100,000, subscript q is an integer from 1 to 50, R¹ is H or CH₃, and R² is selected from O, N, and S.

In a twentieth embodiment, which optionally includes any one or more or each of the first through ninth embodiments and/or the fourteenth embodiment, the polymer has a structure selected from:

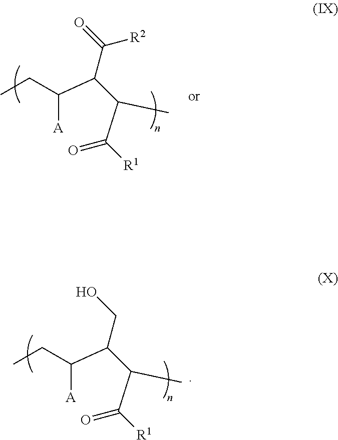

In examples according to the twentieth embodiment, A is, independently in each instance, selected from the group consisting of aryl, alkyl-aryl; heteroalkyl-aryl; heteroaryl, alkyl-heteroaryl, and heteroalkyl-heteroaryl, and subscript n is, independently in each instance, an integer from 1 to 10,000.

In examples according to the twentieth embodiment, R¹ and R² are each, individually in each instance, selected from:

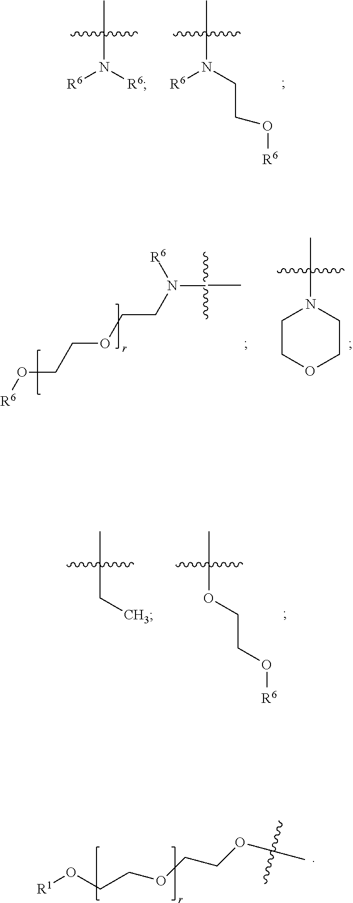

In such examples, subscript r is an integer from 1 to 50, R⁶ is either H or CH₃, and

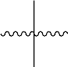

is point of attachment to structure IX or X.

In such an example, A is selected from:

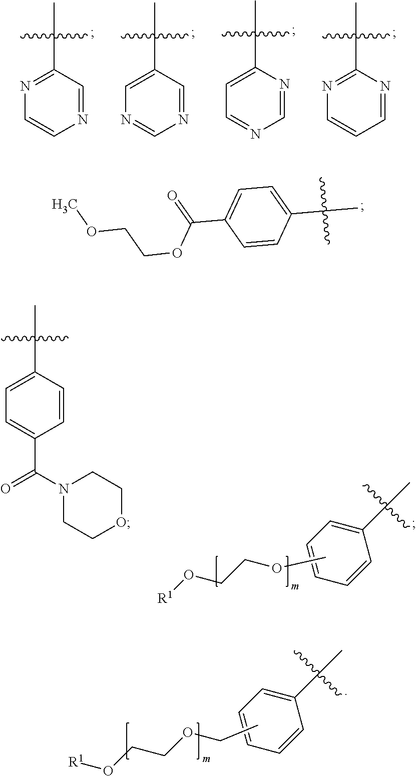

In such an example, subscript m is an integer from 1 to 10, and R¹ is H or CH₃.

In a twenty-first embodiment, which optionally includes any one or more or each of the first through eighth embodiments and/or the fourteenth embodiment, the polymer has a structure selected from:

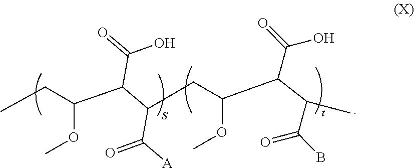

In examples according to the twenty-first embodiment, A and B are each, independently in each instance, selected from the group consisting of aryl, alkyl-aryl; heteroalkyl-aryl; heteroaryl, alkyl-heteroaryl, and heteroalkyl-heteroaryl, and subscript s and t are each, independently in each instance, an integer from 1 to 10,000.

In such an example, A is selected from:

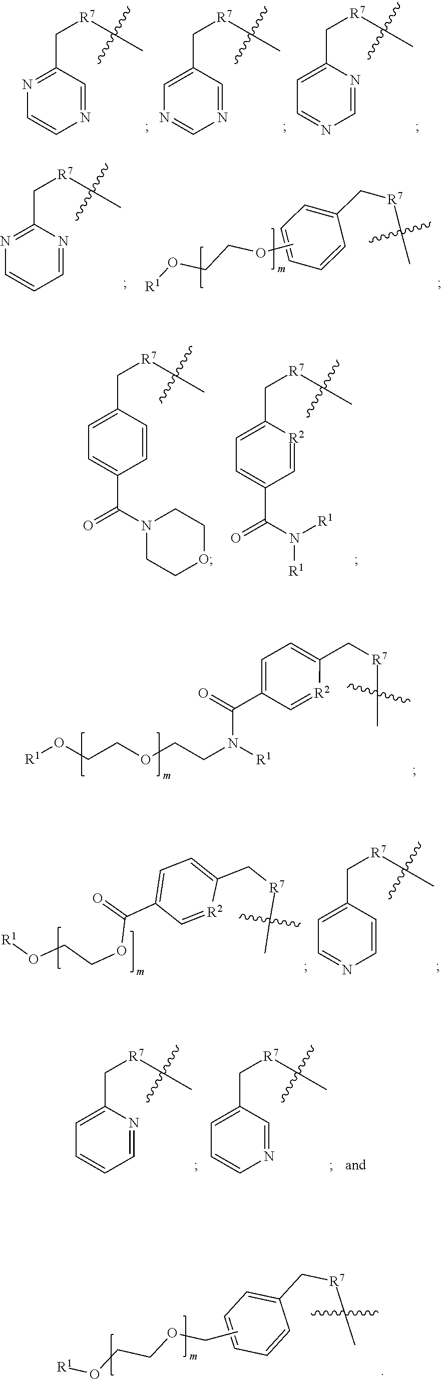

In such an example, subscript m is an integer from 1 to 10, R¹ is H or CH₃, R² is CH or N, and R⁷ is O, NH, or NCH₃.

In a twenty-second embodiment, which optionally includes any one or more or each of the first through ninth embodiments, the polymer has a structure selected from:

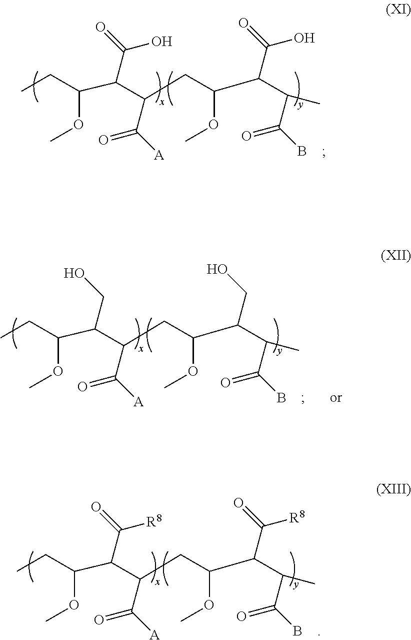

In examples according to the twenty-second embodiment, subscripts u and v are each, independently in each instance, an integer from 1 to 100,000.

In examples according to the twenty-second embodiment, A and B are each, independently in each instance, selected from the group consisting of aryl, alkyl-aryl, heteroalkyl-aryl; heteroaryl, alkyl-heteroaryl, and heteroalkyl-heteroaryl.

In examples according to the twenty-second embodiment, R⁸ is selected from:

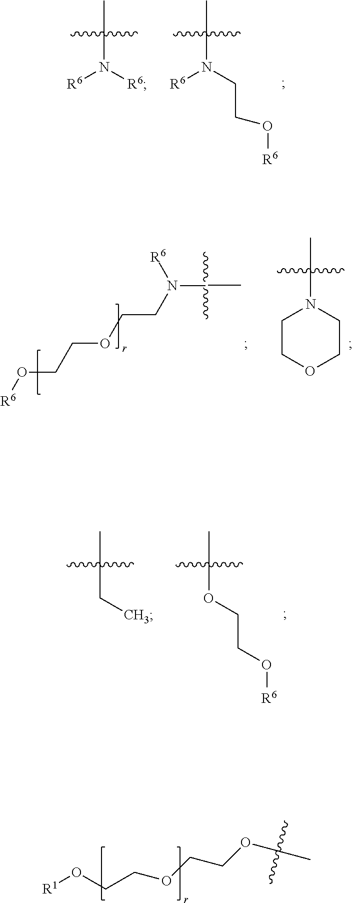

In such an example, subscript m is an integer from 1 to 10, R¹ is either H or CH₃, and

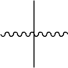

is point of attachment to structure.

In a twenty-third embodiment, which optionally includes any one or more or each of the first through tenth embodiments, the compounds have the structure:

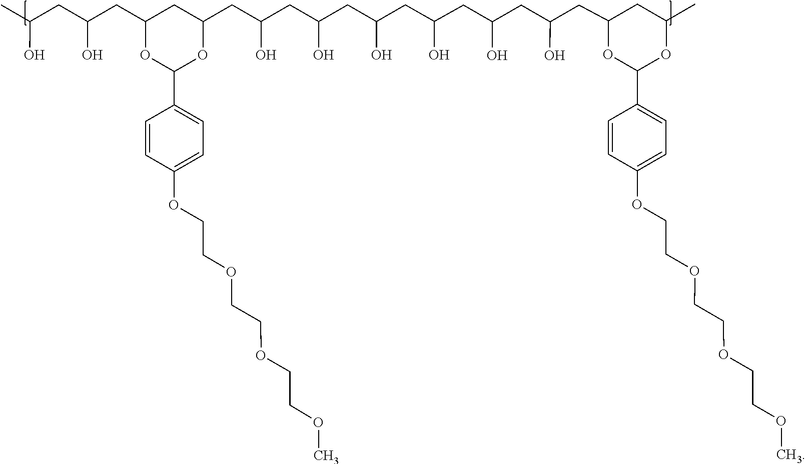

In another aspect, a reaction mixture comprises a dispersing agent and at least one fluorophore, wherein the dispersing agent comprises a polymer according to the first through twenty third embodiments, or combinations thereof.

In embodiments, the fluorophore comprises a polymeric dye.

In embodiments, the fluorophore comprises a polymeric tandem dye.

In embodiments, the fluorophore comprises a tandem dye.

In embodiments of the reaction mixture, the fluorophore is conjugated to a specific binding member that specifically binds to a target analyte. In embodiments, the specific binding member is a biomolecule.

In embodiments, the reaction mixture is in a liquid form. In other embodiments, the reaction mixture is in a dried form. In some examples where the reaction mixture is in a dried form, the dried form comprises a lyophilized form. In some examples where the reaction mixture is in a dried form, the dried form comprises a vitrified form.

In embodiments, the polymer is present at a percent weight-volume (w/v) of the reaction mixture of between 0.001% and 15% w/v, for example at the percent weight-volume of the reaction mixture of between 0.05% and 2% w/v, for example at the percent weight-volume of the reaction mixture of 0.1% and 1% w/v.

In embodiments, the polymer is present in the reaction mixture at a concentration of between 0.1 mg/mL and 100 mg/mL.

In embodiments, the polymer is present in the reaction mixture at a molar ratio of between 50:1 and 1500:1 polymer to specific binding member, respectively. In some examples, the polymer included in the composition is present in the reaction mixture at a molar ratio of between 800:1 and 1100:1 polymer to specific binding member, respectively.

In another aspect, a method of labeling one or more of a target analyte present in a sample comprises contacting the sample with a reaction mixture as described above that includes a fluorophore conjugated to a specific binding member, under conditions sufficient for specific binding of the target analyte to the labeled specific binding member included in the reaction mixture, thereby providing a labeling composition contacted sample.

In embodiments of the method, the sample is a biological sample.

In another aspect, a method of evaluating a sample for the presence and/or amount of a labeled specific binding member-target analyte binding complex, comprises assaying a labeling composition contacted sample as described above for the presence and/or amount of a labeled specific binding member-target analyte binding complex.

In another aspect, a method of preventing aggregation between two or more labeled specific binding members in a solution, comprises providing a solution comprising a dispersing agent comprising a polymer according to the first through twenty third embodiments, or combinations thereof, and separately adding each of the two or more labeled specific binding members to the solution to provide a labeled specific binding member-dispersing agent composition, thereby preventing aggregation between the two or more labeled specific binding members.

In embodiments of such a method, the method further comprises drying down the labeled specific binding member-dispersing agent composition, and, at a later time, reconstituting the labeled specific binding member-dispersing agent composition to provide a reconstituted labeled specific binding member-dispersing agent composition.

In such embodiments, drying down the labeled specific binding member-dispersing agent composition includes lyophilizing or vitrifying the labeled specific binding member-dispersing agent composition.

In embodiments of such a method, preventing aggregation comprises a reduction in aggregation of 80% or more as compared to a case where each of the two or more labeled specific binding members are combined in absence of the dispersing agent. In some embodiments, the reduction in aggregation is 95% or more.

In embodiments of such a method, a molar ratio of a polymer included in the composition to the two or more labeled specific binding members is between 50:1 and 1500:1, respectively, for preventing aggregation.

In embodiments of such a method, a percent w/v of a polymer in the labeled specific binding member-dispersing agent composition, or the reconstituted labeled specific binding member-dispersing agent composition, is between 0.05% and 10% w/v.

In another aspect, a method of reversing aggregation between two or more labeled specific binding members comprises forming a mixture of the two or more labeled specific binding members, wherein forming the mixture results in some level of aggregation between the two or more labeled specific binding members, and contacting the mixture with a dispersing agent comprising a polymer according to any one or more of the first through twenty-third embodiments, or combinations thereof, to provide a labeled specific binding member-dispersing agent composition, thereby reversing aggregation between the two or more labeled specific binding members.

In embodiments of such a method, the mixture of the two or more labeled specific binding members is in liquid form.

In embodiments of such a method, reversing aggregation comprises reversing 80% or more aggregation that occurred between the two or more labeled specific binding members in the mixture prior to the mixture being contacted with the dispersing agent. In embodiments, reversing aggregation comprises reversing 95% or more aggregation.

In embodiments of such a method, a molar ratio of a polymer included in the dispersing agent to the two or more labeled specific binding members is between 50:1 and 1500:1, respectively, in the labeled specific binding member-dispersing agent composition.

In embodiments of such a method, a percent w/v of a polymer in the labeled specific binding member composition is between 0.001% and 15% w/v.

In another aspect, a method of reducing non-specific binding between one or more labeled specific binding members and a cell comprises contacting the cell with a dispersing agent comprising a polymer according to any one or more of the first through twenty-third embodiments, or combinations thereof, prior to and/or during labeling of at least one target analyte associated with the cell with the one or more labeled specific binding members, thereby reducing non-specific binding.

In another aspect, a method of reducing aggregation of a microsphere population comprises contacting the microsphere population with a dispersing agent comprising a polymer according to any one or more of the first through twenty-third embodiments, or combinations thereof, thereby reducing aggregation of the microsphere population as compared to an amount of aggregation of the microsphere population in absence of said dispersing agent.

In embodiments of such a method, the microsphere population comprises microspheres comprised of a hydrophobic polymer.

In embodiments of such a method, the reduction in aggregation occurs in response to contacting the microsphere population with the dispersing agent prior to aggregation of the microsphere population or following aggregation of the microsphere population.

In embodiments of such a method, microspheres corresponding to the microsphere population have diameters ranging from about 5 nm to about 100 μm.

In embodiments of such a method, microspheres corresponding to the microsphere population are internally and/or externally labeled.

In another aspect, a reaction mixture comprises a population of microspheres, and a composition according to any one or more of the first through twenty-third embodiments.

In another aspect, a reaction mixture is provided comprising a wetting agent and at least one biological sample, wherein the wetting agent comprises a polymer according to any one of the first through twenty-third embodiments, or combinations thereof. In embodiments, the biological sample comprises at least one cell. In embodiments, the biological sample comprises one or more cells.

In another aspect, a kit comprises any one or more of the reaction mixtures as described above, and optionally, a reconstitution buffer.

In another aspect, a kit comprises a polymer according to any one or more of the first through twenty-third embodiments, or combinations thereof, and at least one labeled specific binding member in a liquid or a dried form.

In an embodiment of such a kit, the kit further comprises a population of microspheres in solution.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an image of a 10% w/v solution of a dispersant of the present disclosure.

FIGS. 2A-2C are plots generated from flow cytometry analysis showing that aggregation of polymer dye conjugates can be reversed (FIG. 2B) and/or prevented (FIG. 2C) by the dispersants of the present disclosure. Polymer dye conjugates used in the experiments included CD4-BV421 (e.g., BD Biosciences, Franklin Lakes, NJ) and CD8-BV-605 (e.g., BD Biosciences, Franklin Lakes, NJ), and cells tested comprised peripheral blood mononuclear cells (PBMCs). In absence of any dispersant, aggregation of polymer dye conjugates is observed (FIG. 2A).

FIGS. 2D-2G illustrate additional data generated from flow cytometry where the biological sample comprised PBMCs. FIGS. 2D-2E depict data obtained in absence of any dispersant, and FIGS. 2F-2G depict data obtained in presence of dispersant. FIG. 2D and FIG. 2F are scatter plots illustrating collected lymphocytes, and FIG. 2E and FIG. 2G are plots showing the lymphocytes from FIG. 2D and FIG. 2F, respectively, labeled with CD4-BV421 and/or CD8-BV605.

FIGS. 2H-2K illustrate additional data generated from flow cytometry where the biological sample comprised whole blood. FIGS. 2H-2I depict data obtained in absence of any dispersant, and FIGS. 2J-2K depict data obtained in presence of dispersant. FIG. 2H and FIG. 2J are scatter plots illustrating collected lymphocytes, and FIG. 2I and FIG. 2K are plots showing lymphocytes from FIG. 2H and FIG. 2J, respectively, labeled with CD4-BV421 and/or CD8-BV605.

FIGS. 3A-3D are plots generated from flow cytometry showing that aggregation of polymer dye conjugates can be prevented by the dispersants of the present disclosure for at least 14 days. Polymer dye conjugates used in the experiments included CD4-BV421 and CD8-BV-605, and cells tested comprised peripheral blood mononuclear cells (PBMCs). No aggregation was observed when polymer dye conjugates were incubated for 3 days (FIG. 3A), 5 days (FIG. 3B), 7 days (FIG. 3C), or 14 days (FIG. 3D) in the presence of a dispersant (e.g., as herein described), prior to cells being labeled with the polymer dye conjugates.

FIGS. 4A-4B are flow cytometry scatter plots of lymphocytes collected in buffers lacking (FIG. 4A) or containing (FIG. 4B) a wetting agent of the present disclosure. The data illustrate that wetting agents of the present disclosure can increase recovery of cells after multiple wash steps.

FIG. 4C is a graph that plots number of lymphocytes detected for varying conditions (PBS only, PBS+wetting agent (PDD) spike, and PBS+wetting agent (PDD)) as a function of cells fully stained, singly stained, or unstained. polymer dye conjugates used in the experiments included CD4-BV421 and CD8-BV605. The data illustrate that dispersants of the present disclosure increase cell recovery as analyzed by FACS.

FIGS. 5A-5E are flow cytometry plots illustrating that polymer dye conjugates and dispersants (e.g., as herein described) can be vitrified, reconstituted, and used for cellular labeling without any substantial resultant dye-dye aggregation. FIGS. 5A-5B illustrate data obtained where the biological sample comprised PBMCs, and FIGS. 5D-5E illustrate data obtained where the biological sample comprised whole blood. FIG. 5A is a scatter plot showing collected lymphocytes. FIG. 5B is a plot showing the lymphocytes from FIG. 5A labeled with CD4-BV421 and/or CD8-BV605. FIG. 5C is a plot that shows cells fully stained with CD4-BV421 and CD8-BV605, overlaid with CD8-BV605 and CD4-BV421 single color controls. FIG. 5D is a scatter plot showing collected lymphocytes, and FIG. 5E is a plot showing the lymphocytes from FIG. 5D labeled with CD4-BV421 and/or CD8-BV605.

FIG. 6 shows an ultraviolet-visible light (UV-VIS) spectra from Example 7.

FIGS. 7A-7C are plots generated from flow cytometry analysis showing that aggregation of polymer dye conjugates can be reversed (FIG. 7B) and/or prevented (FIG. 7C) by the dispersants of the present disclosure. Polymer dye conjugates used in the experiments included CD8-BUV496 (e.g., BD Biosciences, Franklin Lakes, NJ) and CD19-BUV805 (e.g., BD Biosciences, Franklin Lakes, NJ), and cells tested comprised fresh whole blood. In absence of any dispersant, aggregation of polymer dye conjugates is observed (FIG. 7A).

FIGS. 8A-8B are plots generated from flow cytometry analysis showing that aggregation of polymer dye conjugates can be prevented (FIG. 8B) by the dispersants of the present disclosure. Polymer dye conjugates used in the experiments included CD4-BV421 (e.g., BioLegend, San Diego, CA) and CD56-BV711 (e.g., BioLegend, San Diego, CA), and cells tested comprised fresh whole blood. In absence of any dispersant, aggregation of polymer dye conjugates is observed (FIG. 8A).

FIG. 9 shows liquid chromatography mass spectroscopy (LC-MS) results from Example 10.

FIG. 10 shows liquid chromatography mass spectroscopy (LC-MS) results from Example 10.

FIG. 11 shows liquid chromatography mass spectroscopy (LC-MS) results from Example 10.

FIG. 12 shows liquid chromatography mass spectroscopy (LC-MS) results from Example 10.

### DETAILED DESCRIPTION

### I. Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

“About” as used herein when referring to a measurable value such as an amount, a temporal duration, and the like, is meant to encompass variations of 20% or 110%, more preferably ±5%, even more preferably 10%, and still more preferably ±0.1% from the specified value, as such variations are appropriate to perform the disclosed methods.

As used herein, “w/v” refers to the weight of the component in a given volume of solution.

“Ranges”: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Antibody” as referenced herein is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)₂ and Fv) so long as they exhibit binding activity or affinity for a selected antigen.

As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (I), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

The term antibody is meant to include full length antibodies and may refer to a natural antibody from any organism, an engineered antibody, or an antibody generated recombinantly for experimental, therapeutic, or other purposes as further defined below. Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions.

“Antigen” as used herein refers to any substance capable of eliciting an immune response.

As used herein, the term “alkyl” by itself or as part of another substituent refers to a saturated branched or straight-chain monovalent hydrocarbon radical derived by the removal of one hydrogen atom from a single carbon atom of a parent alkane. Alkyl groups of interest include, but are not limited to, methyl; ethyl, n-propyl, i-propyl, n-butyl, i-butyl, t-butyl. In some embodiments, an alkyl group includes from 1 to 20 carbon atoms. In some embodiments, an alkyl group includes from 1 to 10 carbon atoms. In certain embodiments, an alkyl group includes from 1 to 6 carbon atoms, such as from 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Alkyl substituents may be substituted with 1 to 6 substituents which are not further substituted. The group may be a monovalent terminal group or a divalent bridging group.

“Heteroalkyl” refers to a straight- or branched-chain alkyl group preferably having from 2 to 14 carbons, more preferably 2 to 10 carbons in the chain, one or more of which has been replaced by a heteroatom selected from S, O, P and N. Exemplary heteroalkyls include alkyl ethers (i.e., alkoxyl), secondary and tertiary alkyl amines, amides, alkyl sulfides (i.e., thiol), and the like. Heteroalkyl substituents may be substituted with 1 to 6 substituents which are not further substituted. The group may be a monovalent terminal group or a divalent bridging group.

“Aryl” by itself or as part of another substituent refers to a monovalent aromatic hydrocarbon radical derived by the removal of one hydrogen atom from a single carbon atom of an aromatic ring system. Aryl groups of interest include, but are not limited to, benzyl, napthyl, and anthracyl. In certain embodiments, an aryl group includes from 6 to 20 carbon atoms. In certain embodiments, an aryl group includes from 6 to 12 carbon atoms. Examples of an aryl group are phenyl and naphthyl. Aryl substituents may be substituted with 1 to 6 substituents which are not further substituted.

“Heteroaryl” by itself or as part of another substituent, refers to a monovalent heteroaromatic radical derived by the removal of one hydrogen atom from a single atom of a heteroaromatic ring system. Heteroaromatic includes aromatic compounds in which one or more carbons are substituted with O, N, S, or a combination thereof. Heteroaryl groups of interest include, but are not limited to, furanyl, pyrrolyl, indolyl, isoindolyl, thiophenyl, benzothiophenyl, imidazolyl, benzoimidazolyl, pyrazolyl, pyridyl, pyrimidinyl, pyridazinyl, triazinyl, thiazolyl, benzothiazolyl, and quinolinyl. In certain embodiments, the heteroaryl group is from 5-20 membered heteroaryl. In certain embodiments, the heteroaryl group is from 5-10 membered heteroaryl. Heteroaryl substituents may be substituted with 1 to 6 substituents which are not further substituted.

“Alkyl-aryl” means aryl as defined above which is bonded through a divalent alkyl group to the structure to which the alkyl-aryl is a substituent.

“Aryl-alkyl” means alkyl as defined above which is bonded through a divalent aryl group to the structure to which the aryl-alkyl is a substituent.

“Heteroalkyl-aryl” means aryl as defined above which is bonded through a divalent heteroalkyl group to the structure to which the heteroalkyl-aryl is a substituent.

“Aryl-heteroalkyl” means heteroalkyl as defined above which is bonded through a divalent aryl group to the structure to which the aryl-heteroalkyl is a substituent.

“Alkyl-heteroaryl” means heteroaryl as defined above which is bonded through a divalent alkyl group to the structure to which the alkyl-heteroaryl is a substituent.

“Heteroaryl-alkyl” means alkyl as defined above which is bonded through a divalent heteroaryl group to the structure to which the heteroaryl-alkyl is a substituent.

“Heteroalkyl-heteroaryl” means heteroaryl as defined above which is bonded through a divalent heteroalkyl group to the structure to which the heteroalkyl-heteroaryl is a substituent.

“Heteroaryl-heteroalkyl” means heteroalkyl as defined above which is bonded through a divalent heteroaryl group to the structure to which the heteroaryl-heteroalkyl is a substituent.

As used herein, the term “dispersant” refers to any one or more of the polymers of the present disclosure. The terms “dispersant” and “dispersing agent” are used interchangeably. In embodiments, the dispersing agents of the present disclosure comprise polymers substituted with at least one or more aromatic side groups connected via a saturated hydrocarbon chain. For example, a dispersing agent as herein disclosed contains two or more aromatic groups, for example five or more aromatic groups. Dispersants, as referred to herein, exhibit negligible absorbance or emissive properties at or above 350 nm. The dispersants have a water solubility of at least 1 mg/mL, for example at least 10 mg/mL, for example at least 100 mg/mL. The dispersants exhibit an average molecular weight between 500-5,000,000 Daltons, for example average molecular weight is between 1000-100,000 Daltons, for example between 1,000 and 10,000 Daltons. The aromatic subunits of dispersants of the present disclosure are characterized in that they are soluble in water at ≥1 mg/mL, and do not exhibit a critical micelle concentration (CMC). The dispersants can be completely non-ionic at neutral pH but need not necessarily be. Broadly speaking, dispersing agents as herein disclosed are substances that function to encourage separation of solid or liquid particles, particularly in solutions.

As used herein, the term “wetting agent” refers to an agent that reduces surface tension of a formulation comprising the wetting agent. A wetting agent of the present disclosure comprises and one or more of the polymers as described herein substituted with at least one or more aromatic side groups connected via a saturated hydrocarbon chain, as discussed above. Thus, it may be understood that the polymers of the present disclosure can act as wetting agents and/or as dispersing agents.

As used herein, the term “micelle” refers to a particle comprising a core and a hydrophilic shell, wherein the core is held together at least partially, predominantly or substantially through hydrophobic interactions. As used herein, a “critical micelle concentration (CMC)” refers to a concentration of dispersant as described herein, at which micelles spontaneously form. As used herein a “aqueous CMC” or “CMC_{aq}” refers to the concentration of dispersant as described herein, at which micelles spontaneously form, in an aqueous medium.

As used herein, the terms “polyethylene oxide”, “PEO”, “polyethylene glycol”, “PEG” and “PEG moiety” are used interchangeably and refer to a polymeric group including a chain described by the formula (CH₂—CH₂O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, acyloxy, and amido terminal and/or substituent groups. PEG groups that may be adapted for use in the subject multichromophores include those PEGs described by S. Zalipsky in “Functionalized poly(ethylene glycol) for preparation of biologically relevant conjugates”, Bioconjugate Chemistry 1995, 6 (2), 150-165; and by Zhu et al in “Water-Soluble Conjugated Polymers for Imaging, Diagnosis, and Therapy”, Chem. Rev., 2012, 112 (8), pp 4687-4735.

As used herein, the term “vinyl” is interchangeable with the term “ethenyl.” A “vinyl monomer” has the chemical formula of —CH═CH₂.

As used herein, the term “sample” relates to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment, or synthetic or recombinantly generated samples. The term “sample” may also refer to a “biological sample”. As used herein, the term “a biological sample” refers to a whole organism or a subset of its tissues, cells or component parts (e.g., body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

Samples may be obtained from an in vitro source (e.g., a suspension of cells from laboratory cells grown in culture) or from an in vivo source (e.g., a mammalian subject, a human subject, etc.). In some embodiments, the cellular sample is obtained from an in vitro source. In vitro sources include, but are not limited to, prokaryotic (e.g., bacterial, archaeal) cell cultures, environmental samples that contain prokaryotic and/or eukaryotic (e.g., mammalian, protest, fungal, etc.) cells, eukaryotic cell cultures (e.g., cultures of established cell lines, cultures of known or purchased cell lines, cultures of immortalized cell lines, cultures of primary cells, cultures of laboratory yeast, etc.), tissue cultures, and the like.

In some embodiments, the sample is obtained from an in vivo source and can include samples obtained from tissues (e.g., cell suspension from a tissue biopsy, cell suspension from a tissue sample, etc.) and/or body fluids (e.g., whole blood, fractionated blood, plasma, serum, saliva, lymphatic fluid, interstitial fluid, etc.). In some cases, cells, fluids, or tissues derived from a subject are cultured, stored, or manipulated prior to evaluation. In vivo sources include living multi-cellular organisms and can yield non-diagnostic or diagnostic cellular samples.

In certain embodiments the source of the sample is a “mammal” or “mammalian”, where these terms are used broadly to describe organisms which are within the class Mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

As used herein, the terms “affinity” and “avidity” have the same meaning and may be used interchangeably herein. “Affinity” refers to the strength of binding, increased binding affinity being correlated with a lower K_{d}.

As used herein, the term “biomolecule” refers to an organic molecule or macromolecule of a naturally occurring class of molecules, or a derivative thereof. Biomolecule is meant to encompass polypeptides (e.g., a peptide, an antibody or an antibody fragment), polynucleotides, carbohydrates (e.g., sugars) and lipids. In some cases, the biomolecule is a specific binding member (e.g., as described herein).

As used herein, the term “polypeptide” refers to a polymeric form of amino acids of any length, including peptides that range from 2-50 amino acids in length and polypeptides that are greater than 50 amino acids in length. The terms “polypeptide” and “protein” are used interchangeably herein. The term “polypeptide” includes polymers of coded and non-coded amino acids, chemically or biochemically modified or derivatized amino acids, and polypeptides having modified peptide backbones in which the conventional backbone has been replaced with non-naturally occurring or synthetic backbones. A polypeptide may be of any convenient length, e.g., 2 or more amino acids, such as 4 or more amino acids, 10 or more amino acids, 20 or more amino acids, 50 or more amino acids, 100 or more amino acids, 300 or more amino acids, such as up to 500 or 1000 or more amino acids. “Peptides” may be 2 or more amino acids, such as 4 or more amino acids, 10 or more amino acids, 20 or more amino acids, such as up to 50 amino acids. In some embodiments, peptides are between 5 and 30 amino acids in length.

As used herein, the term “isolated” refers to a moiety of interest that is at least 60% free, at least 75% free, at least 90% free, at least 95% free, at least 98% free, and even at least 99% free from other components with which the moiety is associated with prior to purification.

A “plurality” contains at least 2 members. In certain cases, a plurality may have 10 or more, such as 100 or more, 1000 or more, 10,000 or more, 100,000 or more, 10⁶ or more, 10⁷ or more, 10⁸ or more or 10⁹ or more members.

As used herein, the term “specific binding” refers to the ability of a capture agent (or a first member of a specific binding pair) to preferentially bind to a particular analyte (or a second member of a specific binding pair) that is present, e.g., in a homogeneous mixture of different analytes. In some instances, a specific binding interaction will discriminate between desirable and undesirable analytes in a sample with a specificity of 10-fold or more for a desirable analyte over an undesirable analytes, such as 100-fold or more, or 1000-fold or more. In some cases, the affinity between a capture agent and analyte when they are specifically bound in a capture agent/analyte complex is at least 10⁻⁸ M, at least 10⁻⁹ M, such as up to 10⁻¹⁰ M.

As used herein, the terms “light harvesting multichromophore”, “polymeric dye” and “conjugated polymer” are used interchangeably and refer to a conjugated polymer which has a structure capable of harvesting light with a particular absorption maximum wavelength and converting it to emitted light at a longer emission maximum wavelength. In some cases, the light harvesting multichromophore is itself fluorescent. Conjugated polymers (CPs) are characterized by a delocalized electronic structure and may have an effective conjugation length that is substantially shorter than the length of the polymer chain, because the backbone may contain a large number of conjugated segments in close proximity. In some cases, conjugated polymers are efficient for light harvesting and provide for optical amplification via Forster energy transfer to an acceptor. In embodiments, the light harvesting multichromophore is water soluble.

A multichromophore may be of any convenient molecular weight (MW). In some cases, the MW of the multichromophore may be expressed as an average molecular weight. In some instances, the polymeric dye has an average molecular weight of from 500 to 500,000, such as from 1,000 to 100,000, from 2,000 to 100,000, from 10,000 to 100,000 or even an average molecular weight of from 50,000 to 100,000.

A multichromophore may have one or more desirable spectroscopic properties, such as a particular absorption maximum wavelength, a particular emission maximum wavelength, extinction coefficient, quantum yield, narrow band spectral features, low energy absorption bands, and the like.

In certain embodiments, a multichromophore has narrow band spectral features. A narrow band spectral feature refers to an absorbance or emission spectra with a full width at half maximum (FWHM) of 50 nm or less with peaks centered at 500 nm or more. In some embodiments, the dye has low energy absorption bands having a bandwidth of 200 nm or less, such as 150 nm or less, 100 nm or less, 90 nm or less, 80 nm or less, 70 nm or less, 60 nm or less, 50 nm or less, 40 nm or less, 30 nm or less, 20 nm or less, or even less. In some cases, the bandwidth is determined via a full width at half maximum (FWHM) measurement. In certain embodiments, the dye has low energy absorption bands having a bandwidth of 50 nm or less.

In some embodiments, the multichromophore has an absorption maximum wavelength in the range of 300 to 900 nm, such as 350 to 850 nm, 350 to 600 nm, 360 to 500 nm, 370 to 500 nm, 380 to 500 nm, 390 to 500 nm or 400 to 500 nm, where specific examples of absorption maxima of interest include, but are not limited to: 590 nm, 630 nm, 650 nm, 680 nm and 750 nm. In certain embodiments, the multichromophore has an absorption maximum wavelength of 590 nm±5 nm, 630 nm±5 nm, 650 nm±5 nm, 680 nm±5 nm or 750 nm±5 nm. In some embodiments, the multichromophore has an emission maximum wavelength in the range of 300 to 900 nm, such as 350 to 850 nm, 350 to 600 nm, 360 to 500 nm, 370 to 500 nm, 380 to 500 nm, 390 to 500 nm or 400 to 500 nm, where specific examples of emission maxima of interest include, but are not limited to: 605 nm, 650 nm, 680 nm, 700 nm and 805 nm. In certain embodiments, the multichromophore has an emission maximum wavelength of 605 nm±5 nm, 650 nm±5 nm, 680 nm±5 nm, 700 nm±5 nm or 805 nm±15 nm.

A multichromophore used in the present invention can be charge neutral, cationic or anionic. In some embodiments the multichromophores are polycationic multichromophores.

In embodiments, the multichromophore is comprised of at least one of the structures, or is defined by at least one of the formulas, disclosed in U.S. Pat. Nos. 9,719,998, 10,851,212, 8,110,673, 8,158,444, 8,575,303, 8,575,303, 10,481,161, 10,955,417, 10,605,813, 10,948,485 or 8,354,239, the contents of which are incorporated herein in their entirety.

In embodiments, the multichromophore can include but is not limited to BB515 (BD Biosciences, Franklin Lakes, NJ), BB700 (BD Biosciences, Franklin Lakes, NJ), BUV395 (BD Biosciences, Franklin Lakes, NJ), BUV496 (BD Biosciences, Franklin Lakes, NJ), BUV563 (BD Biosciences, Franklin Lakes, NJ), BUV615 (BD Biosciences, Franklin Lakes, NJ), BUV661 (BD Biosciences, Franklin Lakes, NJ), BUV737 (BD Biosciences, Franklin Lakes, NJ), BUV805 (BD Biosciences, Franklin Lakes, NJ), BV421 (BD Biosciences, Franklin Lakes, NJ), BV480 (BD Biosciences, Franklin Lakes, NJ), BV510 (BD Biosciences, Franklin Lakes, NJ), BV605 (BD Biosciences, Franklin Lakes, NJ), BV650 (BD Biosciences, Franklin Lakes, NJ), BV711 (BD Biosciences, Franklin Lakes, NJ), BV750 (BD Biosciences, Franklin Lakes, NJ), BV786 (BD Biosciences, Franklin Lakes, NJ), SB436 (Thermofisher, Waltham, MA), SB600 (Thermofisher, Waltham, MA), SB645 (Thermofisher, Waltham, MA), SB702 (Thermofisher, Waltham, MA), SB780 (Thermofisher, Waltham, MA), BV570 (BD Biosciences, Franklin Lakes, NJ), BV785 (BD Biosciences, Franklin Lakes, NJ).

In some embodiments, the light harvesting multichromophore is a polymeric tandem dye. Polymeric tandem dyes include two covalently linked moieties: a donor light harvesting multichromophore (e.g., as described herein) and an acceptor chromophore. In some instances, the acceptor chromophore is a quencher. In certain instances, the acceptor chromophore is a fluorescent dye. As used herein, the term “acceptor chromophore” refers to a light-absorbing molecule that is capable of receiving or absorbing energy transferred from the multichromophore. In some cases, the acceptor chromophore can either emit as light the energy received from the multichromophore or dissipate the energy as heat. For example, as used herein, the term “quencher” refers to an acceptor chromophore that absorbs energy from the multichromophore and does not emit light but rather can dissipate the energy as heat.

In some embodiments, the polymeric tandem dye may be excited at the absorption maximum wavelength of the donor multichromophore and may emit light at the emission wavelength of the acceptor chromophore. In some cases, the light-harvesting multichromophore can transfer energy to an acceptor chromophore species in energy-receiving proximity. Mechanisms for energy transfer include, for example, resonant energy transfer (e.g., Forster (or fluorescence) resonance energy transfer, FRET), quantum charge exchange (Dexter energy transfer) and the like. In some instances, these energy transfer mechanisms are relatively short range; that is, close proximity of the light harvesting multichromophore system to the acceptor chromophore provides for efficient energy transfer. In some instances, under conditions for efficient energy transfer, amplification of the emission from the acceptor chromophore occurs when the number of individual chromophores in the light harvesting multichromophore system is large; that is, the emission from the signaling chromophore is more intense when the incident light (the “pump light”) is at a wavelength which is absorbed by the light harvesting multichromophore than when the signaling chromophore is directly excited by the pump light.

By “efficient” energy transfer is meant 30% or more of the energy harvested is transferred to the acceptor. When the acceptor chromophore is a fluorescent dye, the term efficient energy transfer refers to a fluorescent quantum yield of 0.3 or more, such as 0.4 or more, 0.5 or more, or even greater. By “amplification” is meant that the signal from the acceptor chromophore is 1.5× or greater when excited by the light harvesting chromophore as compared to direct excitation with incident light of an equivalent intensity. The signal may be measured using any convenient method. In some cases, the 1.5× or greater signal refers to an intensity of emitted light. In certain cases, the 1.5× or greater signal refers to an increased signal to noise ratio. In certain embodiments of the polymeric tandem dye, the acceptor chromophore emission is 1.5 fold greater or more when excited by the multichromophore as compared to direct excitation of the acceptor chromophore with incident light.

In some instances, the polymeric tandem dye has an extinction coefficient of 5×10⁵ cm⁻¹M⁻¹ or more, such as 6×10⁵ cm⁻¹M⁻¹ or more, 7×10⁵ cm⁻¹M⁻¹ or more, 8×10⁵ cm⁻¹M⁻¹ or more, 9×10⁵ cm⁻¹M⁻¹ or more, such as 1×10⁶ cm⁻¹M⁻¹ or more, 1.5×10⁶ cm⁻¹M⁻¹ or more, 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In some embodiments, the polymeric tandem dye has a molar extinction coefficient of 5×10⁵ M⁻¹cm⁻¹ or more. In certain embodiments, the polymeric tandem dye has a molar extinction coefficient of 1×10⁶ M⁻¹cm⁻¹ or more.

In certain embodiments, the polymeric tandem dye has a quantum yield of 0.3 or more, such as 0.35 or more, 0.4 or more, 0.45 or more, 0.5 or more, 0.55 or more, 0.6 or more, 0.65 or more, 0.7 or more, or even more. In certain cases, the polymeric tandem dye has a quantum yield of 0.4 or more. In certain instances, the polymeric tandem dye has a quantum yield of 0.5 or more.

Any convenient fluorescent dyes may be utilized in the polymeric tandem dyes as an acceptor chromophore. The terms “fluorescent dye” and “fluorophore” are used interchangeably herein. In some embodiments, the acceptor chromophore is a cyanine dye, a xanthene dye, a coumarin dye, a thiazine dye or an acridine dye. Fluorescent dyes of interest include, but are not limited to, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, conjugates thereof, and combinations thereof. Lanthanide chelates of interest include, but are not limited to, europium chelates, terbium chelates and samarium chelates. In some embodiments, the polymeric tandem dye includes a polymeric dye linked to an acceptor fluorophore selected from Cy3, Cy3.5, Cy5, Cy5.5, Cy7, Alexa488, Alexa 647 and Alexa700. In certain embodiments, the polymeric tandem dye includes a polymeric dye linked to an acceptor fluorophore selected from Dyomics dyes (such as DY 431, DY 485XL, DY 500XL, DY 530, DY 610, DY 633, DY 640, DY 651, DY 654, DY 682, DY 700, DY 701, DY 704, DY 730, DY 731, DY 732, DY 734, DY 752, DY 754, DY 778, DY 782, DY 800 or DY 831), Biotium CF 555, Cy 3.5, and diethylamino coumarin.

In some embodiments, the polymeric tandem dye has an absorption maximum wavelength in the range of 300 to 400 nm and an emission maximum wavelength in the range of 375 to 900 nm.

In some embodiments, the polymeric tandem dye is a photostable polymeric tandem dye. A photostable polymeric tandem dye includes a donor water soluble light harvesting multichromophore and a covalently linked luminescent metal complex acceptor in energy-receiving proximity to the multichromophore. The number of luminescent metal complex acceptor units that are linked to the donor water soluble light harvesting multichromophore may vary, where in some instances the number ranges from 1 mol % to 50 mol %, such as from 5 mol % to 25 mol % or from 10 mol % to 25 mol %. In some cases, the luminescent metal complex acceptor is more photostable than an organic fluorophore dye (e.g., a dye that lacks a metal ion such as a xanthene or a cyanine dye). Photodegradation refers to the photochemical modification of a fluorescent molecule leading to a modified molecule with different fluorescence properties (e.g., reduced fluorescence at wavelengths of interest). The water soluble light harvesting multichromophore is itself fluorescent and capable of transferring energy to a linked luminescent metal complex acceptor. As such, excitation of the multichromophore donor leads to energy transfer to and emission from the photostable covalently attached metal complex acceptor.

As used herein, the term “photostable” refers to a fluorescent molecule that is resistant to photodegradation thereby having an emission signal that is substantially stable during exposure to incident excitation light for an extended period of time, e.g., an emission signal that maintains at least 50% intensity for 20 minutes or more (e.g., 40 minutes or more, 50 minutes or more, 60 minutes or more, 90 minutes or more, 2 hours or more, 3 hours or more, 6 hours or more, or even more). In some cases, the exposure of the photostable dye to incident excitation light is continuous. In certain instances, the exposure of the photostable dye to incident excitation light is discontinuous. By “incident excitation light” is meant light having a wavelength and intensity suitable for exciting the light harvesting multichromophore. By “maintains at least X % intensity” is meant that the intensity of the emission signal of the irradiated dye at a given time is at least X % of the emission signal intensity at time zero under the same excitation conditions, where X % can refer to any convenient % intensity from 1 to 100% intensity, e.g., 50% intensity.

In some embodiments, a photostable polymeric tandem dye has an emission signal that maintains at least 60% intensity for 20 minutes or more, such as for 30 minutes or more, 40 minutes or more, 50 minutes or more, 60 minutes or more, 90 minutes or more, 2 hours or more, 3 hours or more, 6 hours or more, or even more. In some instances, a photostable polymeric tandem dye has an emission signal that maintains at least 70% intensity for 20 minutes or more, such as for 30 minutes or more, 40 minutes or more, 50 minutes or more, 60 minutes or more, 90 minutes or more, 2 hours or more, 3 hours or more, 6 hours or more, or even more. In some cases, a photostable polymeric tandem dye has an emission signal that maintains at least 80% intensity for 20 minutes or more, such as for 30 minutes or more, 40 minutes or more, 50 minutes or more, 60 minutes or more, 90 minutes or more, 2 hours or more, 3 hours or more, 6 hours or more, or even more. In certain cases, a photostable polymeric tandem dye has an emission signal that maintains at least 90% intensity for 20 minutes or more, such as for 30 minutes or more, 40 minutes or more, 50 minutes or more, 60 minutes or more, 90 minutes or more, 2 hours or more, 3 hours or more, 6 hours or more, or even more.

Mechanisms for energy transfer from the fluorescent water soluble light harvesting multichromophore donor to the linked luminescent metal complex acceptor include, for example, resonant energy transfer (e.g., Forster (or fluorescence) resonance energy transfer, FRET), quantum charge exchange (Dexter energy transfer) and the like. In some instances, these energy transfer mechanisms are relatively short range; that is, close proximity of the light harvesting multichromophore system to the acceptor metal complex provides for efficient energy transfer. In some instances, under conditions for efficient energy transfer, amplification of the emission from the acceptor metal complex occurs when the number of individual metal complexes in the light harvesting multichromophore system is large; that is, the emission from the luminescent metal complex (e.g., signaling chromophore) is more intense when the incident light (the “pump light”) is at a wavelength which is absorbed by the light harvesting multichromophore than when the luminescent metal complex is directly excited by the pump light.

By “efficient” energy transfer is meant 10% or more, such as 20% or more or 30% or more, of the energy harvested by the donor is transferred to the acceptor. By “amplification” is meant that the signal from the acceptor chromophore is 1.5× or greater when excited by energy transfer from the donor light harvesting multichromophore as compared to direct excitation with incident light of an equivalent intensity. The signal may be measured using any convenient method. In some cases, the 1.5× or greater signal refers to an intensity of emitted light. In certain cases, the 1.5× or greater signal refers to an increased signal to noise ratio. In certain embodiments of the polymeric tandem dye, the acceptor chromophore emission is 1.5 fold greater or more when excited by the multichromophore as compared to direct excitation of the acceptor chromophore with incident light.

The linked luminescent metal complex emission of the polymeric tandem dye can have a quantum yield of 0.03 or more, such as a quantum yield of 0.04 or more, 0.05 or more, 0.06 or more, 0.07 or more, 0.08 or more, 0.09 or more, 0.1 or more, 0.15 or more, 0.2 or more, 0.3 or more or even more. In some instances, the polymeric tandem dye has an extinction coefficient of 5×10⁵ cm⁻¹M⁻¹ or more, such as 6×10⁵ cm⁻¹M⁻¹ or more, 7×10⁵ cm⁻¹M⁻¹ or more, 8×10⁵ cm⁻¹M⁻¹ or more, 9×10⁵ cm⁻¹M⁻¹ or more, such as 1×10⁶ cm⁻¹M⁻¹ or more, 1.5×10⁶ cm⁻¹M⁻¹ or more, 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In some embodiments, the polymeric tandem dye has a molar extinction coefficient of 5×10⁵ M⁻¹cm⁻¹ or more. In certain embodiments, the polymeric tandem dye has a molar extinction coefficient of 1×10⁶ M⁻¹cm⁻¹ or more.

The subject polymeric tandem dyes provide for photostable fluorescence emissions from luminescent metal complexes that are brighter than the emissions which are possible from such metal complexes in isolation. The linked luminescent metal complex emission of the polymeric tandem dye can have a brightness of 50 mM⁻¹cm⁻¹ or more, such as 60 mM⁻¹cm⁻¹ or more, 70 mM⁻¹cm⁻¹ or more, 80 mM⁻¹cm⁻¹ or more, 90 mM⁻¹cm⁻¹ or more, 100 mM⁻¹cm⁻¹ or more, 150 mM⁻¹cm⁻¹ or more, 200 mM⁻¹cm⁻¹ or more, 250 mM⁻¹cm⁻¹ or more, 300 mM⁻¹cm⁻¹ or more, or even more. In certain instances, the linked luminescent metal complex emission of the polymeric tandem dye has a brightness that is at least 5-fold greater than the brightness of a directly excited luminescent metal complex, such as at least 10-fold greater, at least 20-fold greater, at least 30-fold greater, at least 50-fold greater, at least 100-fold greater, at least 300-fold greater, or even greater than the brightness of a directly excited luminescent metal complex.

In embodiments, a polymeric tandem dye is comprised of at least one of the structures, or is defined by at least one of the formulas, disclosed in U.S. Pat. No. 9,719,998, U.S. Ser. No. 10/851,212, or U.S. Ser. No. 10/472,521, the contents of which are incorporated herein in their entirety.

In embodiments, a polymeric tandem dye comprises one or more of BB515, BB700, BUV395, BUV496, BUV563, BUV615, BUV661, BUV737, BUV805, BV421, BV480, BV510, BV605, BV650, BV711, BV750, BV786, SB436, SB600, SB645, SB702, SB780, BV570, BV785.

As used herein, the term “specific binding member” refers to one member of a pair of molecules which have binding specificity for one another. One member of the pair of molecules may have an area on its surface, or a cavity, which specifically binds to an area on the surface of, or a cavity in, the other member of the pair of molecules. Thus, the members of the pair have the property of binding specifically to each other to produce a binding complex. In some embodiments, the affinity between specific binding members in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. In some embodiments, the specific binding members specifically bind with high avidity. By high avidity is meant that the binding member specifically binds with an apparent affinity characterized by an apparent K_{d} of 10×10⁻⁹ M or less, such as 1×10⁻⁹ M or less, 3×10⁻¹⁰ M or less, 1×10⁻¹⁰ M or less, 3×10⁻¹¹ M or less, 1×10⁻¹¹ M or less, 3×10⁻¹²M or less or 1×10⁻¹² M or less.

Aspects of the present disclosure include labeled specific binding members. A “labeled specific binding member,” as used herein, can be a conjugate of a subject multichromophore (e.g., as described herein) and a specific binding member. The multichromophore may comprise a polymeric dye. The multichromophore comprise a polymeric tandem dye. Any of the multichromophores described herein may be conjugated to a specific binding member. The specific binding member and the multichromophore may be conjugated (e.g., covalently linked) to each other via any convenient locations of the multichromophore, via an optional linker. Broadly speaking, a “labeled specific binding member” as used herein encompasses any specific binding member coupled to a fluorophore (e.g., GFP, fluorescein, and the like), and can even include non-fluorophore labels, for example, enzyme conjugates, mass tags, and the like.

As used herein, the term “proteinaceous” refers to a moiety (e.g., a specific binding member) that is composed of amino acid residues. A proteinaceous moiety may be a polypeptide. In some embodiments, the specific binding member is proteinaceous. In certain cases, the proteinaceous specific binding member is an antibody. In certain embodiments, the proteinaceous specific binding member is an antibody fragment. In certain embodiments, the specific binding member is an antibody. In certain embodiments, the specific binding member is a Fab fragment, a F(ab′)₂ fragment, a scFv, a diabody or a triabody. In some cases, the specific binding member is a murine antibody or binding fragment thereof. In certain instances, the specific binding member is a recombinant antibody or binding fragment thereof. In certain instances, the specific binding member is a chimeric antibody. In certain instances, the specific binding member is a human antibody. In certain instances, the specific binding member is a humanized antibody.

The conjugation of multichromophores (and fluorophores in general) is not limited to dyes or antibodies; rather, the multichromophores described herein can be conjugated to any variety of biomolecules, including proteins (such as avidin/streptavidin), nucleic acids, affinity ligands, sugars, lipids, peptides, and substrates for enzymes.

As used herein, the term “microspheres” refers to small, generally spherical particles with diameters in the nanometer to micrometer range (e.g., 2 nm-100 μm, for example between 5 nm and 1 μm, for example between 20 nm and 500 nm, for example between 50 nm and 250 nm, or for example between 2 nm and 20 nm, such as between 2 nm and 10 nm, or for example between 1-100 μm, for example between 1-50 μm, for example between 1-25 μm, for example between 1-10 μm). The term “microsphere” as used herein is used interchangeably with “microparticle,” and “nanoparticle.” Microspheres in general can be manufactured from various natural and synthetic materials. Polystyrene microspheres are typically used in biomedical applications, for example cell sorting applications and immunoprecipitation applications. For example, polystyrene microspheres may be coated with recognition molecules including but not limited to antibodies, antigens, peptides, and nucleic acid probes, fluorophores, and the like. Polystyrene microspheres can be loaded with dyes and other various compounds. Also encompassed within the term “microspheres” are other polymer microspheres, as well as silica and magnetic microspheres.

Also encompassed within the term “microsphere” according to the present disclosure are quantum dots. As used herein, “quantum dot,” “dot,” and “nanocrystal” are synonymous, and refer to any semiconductor crystal with size dependent optical and electrical properties along at least three orthogonal dimensions. A quantum dot is differentiated from a quantum wire and a quantum well, which are crystals with size-dependent optical and electronic properties along at most two or one dimension respectively.

It will be appreciated by one of skill in the art that quantum dots can exist in a variety of shapes, including but not limited to spheroids, rods, disks, pyramids, cubes and a plurality of alternative geometric and non-geometric shapes. While these shapes can dramatically affect the physical, optical and electronic characteristics of the quantum dot, the specific shape does not bear on the qualification of the crystal as a quantum dot.

For convenience, we herein describe the size of quantum dots in terms of “diameter”. In the case of spherically shaped quantum dots, diameter is used as is commonly understood. For non-spherical quantum dots, the term diameter, unless otherwise defined, refers to the radius of revolution in which the entire non-spherical quantum dot would fit.

A quantum dot will typically comprise a “core” of one or more first materials and can optionally be surrounded by a “shell” of a second material. A quantum dot core surrounded by a shell is referred to as a “core-shell” quantum dot.

The term “core” refers to the inner-portion of the quantum dot such that the core-region is substantially a single homogeneous monoatomic or polyatomic material. The core can be either crystalline, polycrystalline or amorphous. The core may be “defect” free or contain a range of defect densities. In this case, “defect” refers to any crystal stacking error, vacancy, insertion or impurity entity (e.g., dopant) placed within the core-material. Impurities can be either atomic or molecular.

While the core may herein be referred to as “crystalline”, it will be understood by one of skill in the art that the surface of the core may be polycrystalline or amorphous and that this non-crystalline surface may extend a measurable depth within the core. The potentially non-crystalline nature of the “core-surface” does not change what is described herein as a substantially crystalline core. The core-surface region optionally contains defects or impurities. The core-surface region will preferably range in depth between one and five atomic-layers, and may be either substantially homogeneous, substantially inhomogeneous or continuously varying as a function of position within the core-surface region.

Quantum dots may optionally comprise a “shell” of a second material that surrounds the outside of the inner core. A “shell” is a layer of material, either organic or inorganic, that covers the surface of the core region of the quantum dot.

In certain embodiments herein, quantum dots are fluorophores in that they absorb photons of light and then re-emit longer wavelength photons nearly instantaneously. In embodiments, quantum dots as herein discussed are nanometer-scale clusters of semiconductor atoms, optionally coated with an additional semiconductor shell and optionally with a coating (e.g., polymeric coating) to enable coupling to proteins (e.g., antibodies), oligonucleotides, small molecules, and the like, which are in turn used for specific binding of the quantum dots to a target of interest (e.g., a target analyte).

Quantum Dots herein may include any set forth in U.S. Pat. Nos. 10,717,927; 10,611,958; 9,804,319; 8,071,361; 6,207,392; 6,727,065; 7,311,774; 7,851,338; 6,501,091; 6,576,291; 7,125,605; 7,374,824; and 8,481,112. The entire contents of these patents are herein incorporated by reference in their entirety.

The phrase the polymer comprises means that the structure shown may be included as part of another polymer or mixed with other polymers. For example, when the structure is included as part of another polymer, the terminal ends of the polymer may include structures not shown herein so long as the polymer does include portions which have the structures shown herein.

### II. Methods of Use

### A. Methods of Evaluating a Sample

Aspects of the invention include methods of evaluating a sample for the presence of one or more target analytes. Broadly speaking, a method includes (a) contacting a sample with a polymeric dye conjugate preparation that has been contacted with one or more dispersants (e.g., as described herein) to produce a labeling composition contacted sample, and (b) assaying the labeling composition contacted sample for the presence of a polymeric dye conjugate-target analyte binding complex. In embodiments, the polymeric dye conjugate preparation includes two or more different polymeric dye conjugates.

It is also within the scope of this disclosure that the methodology can be used for other dye conjugates that are not polymeric dye conjugates. Discussed herein, a “labeled specific binding member” encompasses a polymeric dye conjugate, but also encompasses, for example a specific binding member labeled with at least one fluorophore that is not coupled to a polymer. Examples include but are not limited to small molecule organic dyes (e.g., fluorescein, Texas Red, Cy3, Cy5, and the like), fluorescent proteins (e.g., GFP, YFP, and the like), tandem dyes (non-polymer) (e.g., PE/Cy5, PE/Cy7, and the like).

Accordingly, in embodiments, a method includes a) contacting a sample with a labeled specific binding member preparation that has been contacted with one or more dispersants (e.g., as described herein) to produce a labeling composition contacted sample, and (b) assaying the labeling composition contacted sample for the presence of a labeled specific binding member-target analyte binding complex. In embodiments, the labeled specific binding member preparation includes two or more different labeled specific binding members (e.g., an antibody conjugated to first fluorophore and another antibody conjugated to a second fluorophore).

### 1. Target Analytes

Any convenient specific binding members may be utilized in the conjugate. Specific binding members of interest include, but are not limited to, those agents that specifically bind cell surface proteins of a variety of cell types, including but not limited to, stem cells, e.g., pluripotent stem cells, hematopoietic stem cells, T cells, T regulator cells, dendritic cells, B Cells, e.g., memory B cells, antigen specific B cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that have a convenient cell surface marker or antigen that may be captured by a convenient specific binding member conjugate. In some embodiments, the target cell is selected from HIV containing cell, a Treg cell, an antigen-specific T-cell populations, tumor cells or hematopoetic progenitor cells (CD34+) from whole blood, bone marrow or cord blood. Any convenient cell surface proteins or cell markers may be targeted for specific binding to labeled specific binding members in the subject methods. In some embodiments, the target cell includes a cell surface marker selected from a cell receptor and a cell surface antigen. In some cases, the target cell may include a cell surface antigen including but not limited to CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, LgrS (N-Terminal), SSEA-3, TRA-1-60 Antigen, Disialoganglioside GD2 and CD71.

Any convenient targets may be selected for evaluation utilizing the subject methods. Targets of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell, a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods or purchased commercially. In some embodiments, the labeled specific binding members comprise an antibody or antibody fragment. Any convenient target analyte that specifically binds an antibody or antibody fragment of interest may be targeted in the subject methods.

In some embodiments, the target analyte is associated with a cell. In certain instances, the target analyte is a cell surface marker of the cell. In certain cases, the cell surface marker is selected from a cell receptor and a cell surface antigen. In some instances, the target analyte is an intracellular target, and the methodology further includes lysing the cell.

In some embodiments, the sample may include a heterogeneous cell population from which target cells are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue. In some cases, the sample includes hematopoetic progenitor cells (e.g., CD34+ cells) in whole blood, bone marrow or cord blood. In certain embodiments, the sample includes tumor cells in peripheral blood. In certain instances, the sample is a sample including (or suspected of including) viral cells (e.g., HIV).

The labeled specific binding members find use in the subject methods, e.g., for labeling a target cell, particle, target or analyte with a labeled specific binding member (e.g., polymeric dye or polymeric tandem dye). For example, labeled specific binding members find use in labeling cells to be processed (e.g., detected, analyzed, and/or sorted) in a flow cytometer. The labeled specific binding members may include antibodies that specifically bind to, e.g., cell surface proteins of a variety of cell types (e.g., as described herein). The labeled specific binding members may be used to investigate a variety of biological (e.g., cellular) properties or processes such as cell cycle, cell proliferation, cell differentiation, DNA repair, T cell signaling, apoptosis, cell surface protein expression and/or presentation, and so forth. Labeled specific binding members may be used in any application that includes (or may include) antibody-mediated labeling of a cell, particle or analyte.

Once the sample has been contacted with the labeled specific binding member, any convenient methods may be utilized in assaying the labeling composition contacted sample that is produced for the presence of a labeled specific binding member-target analyte binding complex. The labeled specific binding member-target analyte binding complex is the binding complex that is produced upon specific binding of the specific binding member of the conjugate to the target analyte, if present. Assaying the labeling composition contacted sample may include detecting a fluorescent signal from the binding complex, if present. In some cases, the assaying includes a separating step where the target analyte, if present, is separated from the sample. A variety of methods may be utilized to separate a target analyte from a sample, e.g., via immobilization on a support. Assay methods of interest include, but are not limited to, any convenient methods and assay formats where pairs of specific binding members such as avidin-biotin or hapten-anti-hapten antibodies find use, are of interest. Methods and assay formats of interest that may be adapted for use with the subject compositions include, but are not limited to, flow cytometry methods, in-situ hybridization methods, enzyme-linked immunosorbent assays (ELISAs), western blot analysis, magnetic cell separation assays and fluorochrome purification chromatography.

### 2. Labeled Specific Binding Member Preparations

Methods of the present disclosure include contacting labeled specific binding member preparations with one or more dispersants as described herein. The dispersants, as herein disclosed, are substantially non-absorbing and non-emitting within the range of 350-850 nm. In embodiments, the dispersants display no absorption and no emission within the range of 350-850 nm.

### 3. Aggregation Reversal

In embodiments, the labeled specific binding member preparation is formulated, and then the one or more dispersants are added to the labeled specific binding member preparation. In this way, dye aggregation that may have occurred during the process of formulation of the labeled specific binding member preparation may be reversed. The one or more dispersants added to the labeled specific binding member preparations may fully reverse aggregation (i.e., 100% reversal), or may reverse dye aggregation to some extent, for example 99% or more, 95% or more, 90% or more, 85% or more, 80% or more, 75% or more, 70% or more, 65% or more, 60% or more, or 50% or more. Following the reversal of any aggregation that occurred prior to the contacting of the labeled specific binding member preparation with the one or more dispersants, the sample may be contacted with the labeled specific binding member preparation. In embodiments, maximum extent of reversal may be realized at some amount of time following the one or more dispersants being added to the labeled specific binding member preparations. For example, maximum extent of reversal may be realized by between 1-20 minutes after the addition of the one or more dispersants to the labeled specific binding member preparations.

In embodiments, a time frame between formulation of the labeled specific binding member preparation, and the contacting of said preparation with the one or more dispersants, can be between 30 seconds and 7 days, or even greater than 7 days, such as 10 days or 15 days. In embodiments, the time frame at which at least 90% reversal, for example 95% reversal or more, of dye aggregation is realized upon addition of the one or more dispersants is less than 1 day, between 1 day and 3 days, between 2 days and 5 days, between 3 days and 7 days.

In embodiments, a time frame between the reversal of aggregation realized via contacting of the one or more dispersants to the labeled specific binding member preparation, and the subsequent contacting of the sample with the labeled specific binding member preparation, may be between 1 minute and 7 days, or even greater than 7 days, for example between 7-10 days, or 10-15 days. During such a time frame that the labeled specific binding member preparation sits in contact with the one or more dispersants prior to contacting the sample, dye aggregation may be prevented fully (e.g., 100%), or to some extent less than 100%, for example 99%, or 95%, or 90%, or 85%, or 80%. Said another way, during such a time frame that the labeled specific binding member preparation sits in contact with the one or more dispersants prior to contacting the sample, the dyes may not aggregate at all, or may exhibit 1% or less aggregation, or may exhibit less than 5% aggregation, or less than 10% aggregation, or less than 15% aggregation, or less than 80% aggregation.

Thus, within the scope of this disclosure is a method of reversing aggregation of labeled specific binding members, the method comprising providing a formulation comprising the labeled specific binding members, and adding one or more dispersants (e.g., as herein described), thereby reversing aggregation between the labeled specific binding members. In embodiments, the labeled specific binding members comprise at least two different labeled specific binding members, in terms of label and/or specific binding member.

### 4. Aggregation Prevention

In embodiments, dispersants as herein described are used in the prevention of dye aggregation before dye aggregation occurs. In such an example, a buffer may be formulated with the one or more dispersants, and then the labeled specific binding member(s) may be added thereto, to form the labeled specific binding member preparation. In this way, dye aggregation may be avoided. Specifically, dye aggregation may be fully prevented (e.g., 100%), 99% prevented or more, 95% prevented or more, 90% prevented or more, 85% prevented or more, 80% prevented or more, 75% prevented or more, 70% prevented or more, 65% prevented or more, or 60% prevented or more, as compared to formulation of a labeled specific binding member preparation in absence of the one or more dispersants.

A time frame in which the labeled specific binding member preparation is held subsequent to the prevention of aggregation, and prior to the contacting of the sample with the labeled specific binding member preparation, may be between 1 minute and 7 days, or even greater than 7 days, for example between 7-10 days, or 10-15 days. Similar to that discussed above, during such a time frame that the labeled specific binding member preparation sits in contact with the one or more dispersants prior to contacting the sample, dye aggregation may be prevented fully (e.g., 100%), or to some extent less than 100%, for example 99%, or 95%, or 90%, or 85%, or 80%. In other words, during such a time frame that the labeled specific binding member preparation sits in contact with the one or more dispersants prior to contacting the sample, the dyes may not aggregate at all, or may exhibit 1% or less aggregation, or may exhibit less than 5% aggregation, or less than 10% aggregation, or less than 15% aggregation, or less than 80% aggregation.

Thus, within the scope of this disclosure is a method of preventing aggregation between labeled specific binding members, the method comprising preparing a formulation that includes at least one dispersant (e.g., as described herein), and adding to the formulation the labeled specific binding members (e.g., as described herein), thereby preventing aggregation between the labeled specific binding members. In embodiments, the labeled specific binding members comprise at least two different labeled specific binding members in terms of label and/or specific binding member.

### 5. Dried Specific Binding Member Preparations

In embodiments, the one or more labeled specific binding members may be lyophilized. As used herein, “lyophilization” refers to a process in which liquid is removed from a product (e.g., cocktail of labeled specific binding members) following the product being frozen and placed in a vacuum, thereby allowing the ice to change directly from solid to vapor without passing through a liquid phase. In embodiments, the one or more labeled specific binding members may be vitrified. As used herein, the term “vitrify” refers to the rapid removal of water molecules in the absence of ice crystal formation by evaporation, possibly vacuum assisted. The process of vitrifying converts the solution into glass or a glasslike substance. The primary differentiation between these processes is that during lyophilization water molecules are sublimed resulting in a porous structure, whereas during vitrification water molecules are evaporated resulting in a non-porous film.

In embodiments, the one or more labeled specific binding members are lyophilized or vitrified in the presence of one or more dispersants. In such an example, a buffer used to lyophilize or vitrify the one of more labeled specific binding members may contain the one or more dispersants.

In embodiments, the one or more labeled specific binding members are lyophilized or vitrified in the absence of the one or more dispersants. In such an example, a buffer used to reconstitute the lyophilized or vitrified product may in some examples contain the one or more dispersants. In other examples, the buffer used to reconstitute the lyophilized or vitrified product may not contain the one or more dispersants. Where the buffer used to reconstitute the product lacks the one or more dispersants, the one or more dispersants may be added following reconstitution, to reverse dye aggregation that occurred during the lyophilization, vitrification and/or reconstitution process. In embodiments where the buffer used to reconstitute the product contains the one or more dispersants, the one or more dispersants may serve to reverse dye aggregation that occurred during the lyophilization or vitrification process and may further serve to prevent any further aggregation that may occur during the reconstitution process.

Suitable drying processes and buffers for use in the subject invention include, e.g., Mattern et al., Formulation of proteins in vacuum-dried glasses. I. Improved vacuum drying of sugars using crystalising amino acids, European J. Pharmaceutics and Biopharmaceutics, 44:177-185 (1997); Mattern et al., Formulation of proteins in vacuum-dried glasses. II. Process and storage stability in sugar-free amino acid systems, Pharmaceutical Development and Technology 4(2):199-208 (1999); Emami et al., Drying technologies for the stability and bioavailability of biopharmaceuticals, Pharmaceutics 2018, 10, 131, doi:10.3390/pharmaceutics10030131; Kumru, et al., Physical characterization and stabilization of a lentiviral vector against absorption and freeze-thaw; Journal of Pharmaceutical Sciences xxx (2018) 1-11, doi:10.1016/j.xphs.2018.07.010; Haeuser et al., Excipients for room-temperature stable freeze-dried monoclonal antibody formulations, Journal of Pharmaceutical Sciences 109:807-817 (2020); US 2015/005669, which published Feb. 26, 2015; US 2018/0363002, which published Dec. 20, 2018; US 2018/0224460, which published Aug. 9, 2018; U.S. Pat. No. 10,538,785, which issued Jan. 21, 2020; U.S. Pat. No. 9,969,984, which issued May 15, 2018; and U.S. Pat. No. 10,724,006, which issued Jul. 28, 2020, all of the disclosures of which are expressly incorporated by reference herein in their entirety.

### 6. Dispersant Amounts

For the prevention or reversal of dye aggregation as herein described, the one or more dispersants are in excess to that of the one or more labeled specific binding members. Specifically, a ratio of dispersant to labeled specific binding member(s) is highly favored toward dispersant. Accordingly, any dispersant absorption/emission within the range of 350-850 nm would dominate as compared to absorption/emission with respect to the labeled specific binding members, hence the dispersants of the present disclosure lack absorption/emission within the range of 350-850 nm.

In embodiments, a dispersant to labeled specific binding member molar ratio is between 50:1 and 1500:1, respectively. For example, a dispersant to labeled specific binding member molar ratio can be 50:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, or any value there between. In some embodiments, the dispersant to labeled specific binding member molar ratio is about 1000:1.

In embodiments, the dispersant is present at a % w/v of between 0.001% and 30%, for example 0.05% w/v, 0.1% w/v, 0.5% w/v, 1% w/v, 4% w/v, or 6% w/v, or 8% w/v, or 10% w/v, or 12% w/v, or 14% w/v, or 16% w/v, or 18% w/v, or 20% w/v, or 22% w/v or 24% w/v, or 26% w/v, or 28% w/v, or 30% w/v. In some embodiments the dispersant is present at a % w/v of between 0.05% and 2% w/v, for example between 0.1% and 1% w/v %.

In embodiments, dispersant concentration in the presence of labeled specific binding member(s) is between 0.001 g/mL and 100 mg/mL. In embodiments, dispersant concentration in the presence of labeled specific binding member(s) is between about 8 mg/mL and 12 mg/mL, for example about 10 mg/mL.

### 7. Buffers

The labeled specific binding member(s) and dispersants of the present disclosure may be prepared in buffered solutions. Buffers of interest include a biological buffer such as phosphate buffers, lactate buffers, etc., including but not limited to N-(2-acetamido)-aminoethanesulfonic acid (ACES), acetate, N-(2-acetamido)-iminodiacetic acid (ADA), 2-aminoethanesulfonic acid (AES), ammonia, 2-amino-2-methyl-1-propanol (AMP), 2-amino-2-methyl-1,3-propanediol (AMPD), N-(1,1-dimethyl-2-hydroxyethyl)-3-amino-2-hydroxypropanesulfonic acid (AMPSO), N,N-bis-(2-hydroxyethyl)-2-aminoethanesulfonic acid (BES), bicarbonate, N,N′-bis-(2-hydroxyethyl)-glycine, [Bis-(2-hydroxyethyl)-imino]-tris-(hydroxymethylmethane) (BIS-Tris), 1,3-Bis[tris(hydroxymethyl)-methylamino]propane (BIS-Tris-propane), boric acid, dimethylarsinic acid, bovine serum albumin (BSA) 3-(Cyclohexylamino)-propanesulfonic acid (CAPS), 3-(cyclohexylamino)-2-hydroxy-1-propanesulfonic acid (CAPSO), carbonate, cyclohexylaminoethanesulfonic acid (CHES), citrate, 3-[N-Bis(hydroxyethyl)amino]-2-hydroxypropanesulfonic acid (DIPSO), formate, glycine, glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, malate, maleate, 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), phosphate, Piperazine-N,N′-bis(2-ethanesulfonic acid) (PIPES), Piperazine-N,N′-bis(2-hydroxypropanesulfonic acid) (POPSO), pyridine, polyvinylpyrrolidone (PVP), succinate, 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), 2-Aminoethanesulfonic acid, AES (Taurine), trehalose, triethanolamine (TEA), 2-[Tris(hydroxymethyl)-methylamino]-ethanesulfonic acid (TES), N-[Tris(hydroxymethyl)-methyl]-glycine (tricine), Tris(hydroxymethyl)-aminomethane (Tris), glyceraldehydes, mannose, glucosamine, mannoheptulose, sorbose-6-phosphate, trehalose-6-phosphate, maleimide, iodoacetates, sodium citrate, sodium acetate, sodium phosphate, sodium tartrate, sodium succinate, sodium maleate, magnesium acetate, magnesium citrate, magnesium phosphate, ammonium acetate, ammonium citrate, ammonium phosphate, among other buffers.

In embodiments, buffer concentration may be used at 5-25 mM. In embodiments where dispersants are suspended in first solution that is then added to a second solution (e.g., to reverse aggregation), the buffers in each of the first solution and the second solution may be the same. In other examples, the buffers in each of the first solution and the second solution may be different, without departing from the scope of this disclosure.

In embodiments, similar buffers may be used for the specific binding of the labeled specific binding member and the target analyte. Solutions used can comprise a balanced salt solution, e.g., normal saline, PBS, Hank's balanced salt solution, etc., optionally supplemented with fetal calf serum, human platelet lysate or other factors, in conjunction with an acceptable buffer at low concentration, such as from 5-25 mM, such as one or more of the above-mentioned buffers. Media for use with the methods as herein disclosed can include but is not limited to dMEM, HBSS, dPBS, RPMI, Iscove's medium, etc. Final components of the media solutions can be selected depending on the components of the sample which are included.

In some embodiments, a particular buffer may be used for drying down labeled specific binding members in combination with, or in the absence of, one or more dispersants as herein disclosed, for purposes of lyophilization or vitrification. Suitable drying buffers include those mentioned hereinabove and will typically include at least one compound that helps stabilize the one or more labeled specific binding members when water molecules are removed by sublimation or evaporation, e.g. trehalose, sucrose, and the like.

### 8. Temperature Dependence of Aggregation Prevention/Reversal

The dispersants as described herein are effective at preventing/reversing dye aggregation at various temperatures, specifically, between temperatures including but not limited to about 0° C. to about 45° C., although temperatures higher than 45° C. are within the scope of this disclosure. In preferred embodiments, effective prevention/reversal of dye aggregation via the dispersants of the present disclosure occurs between about 0-6° C., or from about 12-18° C. (e.g., about 15° C.), or from about 22-28° C. (e.g., about 25° C.), or from about 34-40° C. (e.g., about 37° C.). In embodiments, a rate at which dye aggregation is reversed may be increased with increasing temperatures and may be decreased with decreasing temperatures.

### 9. Temperature Dependence of Labeled Specific Binding Members to Targets

The temperature at which specific binding of the specific binding member of the conjugate to the target analyte takes place may vary, and in some instances may range from 0° C. to 50° C., such as from 10° C. to 40° C., 15° C. to 40° C., 20° C. to 40° C. In embodiments, the temperature may range from about 12-18° C., or from about 22-28° C., or from about 34-40° C. (e.g., about 37° C.), e.g., about 15° C., about 20° C., about 25° C., about 30° C., about 35° C. or about 37° C. In some instances, the temperature at which specific binding takes place is selected to be compatible with the biological activity of the specific binding member and/or the target analyte. In certain instances, the temperature is 25° C., 30° C., 35° C. or 37° C. In certain cases, the specific binding member is an antibody or fragment thereof and the temperature at which specific binding takes place is room temperature (e.g., 25° C.), 30° C., 35° C. or 37° C. (although other temperatures such as those described above are within the scope of this disclosure). Any convenient incubation time for specific binding may be selected to allow for the formation of a desirable amount of binding complex, and in some instances, may be 1 minute (min) or more, such as 2 min or more, 10 min or more, 30 min or more, 1 hour or more, 2 hours or more, or even 6 hours or more.

### 10. Non-Specific Binding

The dispersants of the present disclosure, as used in the methods herein described, can function to reduce non-specific binding of the labeled specific binding members (e.g., polymer dye conjugates) to cells. For example, in a method such as that discussed above that includes contacting a sample with a labeled specific binding member preparation that has been contacted with one or more dispersants (e.g., as described herein) to produce a labeling composition contacted sample, the presence of the one or more dispersants in the labeling composition contacted sample may function to reduce non-specific binding of the labeled specific binding members to the subject cells.

In embodiments, the reduction in non-specific binding is solely due to dispersant carry-over from the labeled specific binding member preparation. As an exemplary illustration, cells of the sample may comprise PBMCs, and the PBMCs may not have been contacted with any dispersant prior to being contacted with the labeled specific binding member preparation (which includes one or more dispersants). In another exemplary illustration, cells of the sample may comprise whole blood, where the whole blood sample may not have been contacted with any dispersant prior to being contacted with the labeled specific binding member preparation (which includes one or more dispersants). Given the high w/v percentage of the one or more dispersants in the labeled specific binding member preparation, upon contacting the sample with the labeled specific binding member preparation the dispersant concentration may remain high enough to additionally function to reduce non-specific binding of the labeled specific binding member(s) to the cells in the sample.

In such examples, reduction of non-specific binding occurs when a dilution factor (i.e., dilution of the labeled specific binding member preparation into the sample) is 1/1, or 1/2, or between 1/2 and 1/5, or between 1/2 and 1/10, or between 1/5 and 1/10, or between 1/10 and 1/15, or between 1/10 and 1/20, or between 1/15 and 1/20, or between 1/20 and 1/40, or between 1/25 and 1/50, or between 1/50 and 1/100, or between 1/100 and 1/1000, or between 1/1000 and 1/2000, or between 1/2000 and 1/10,000, or between 1/10,000 and 1/20,000, or between 1,20,000 and 1/50,000, or between 1/50,000 and 1/100,000.

In embodiments, it is also within the scope of this disclosure that one or more dispersants (e.g., as herein described) can be added to a sample containing cells prior to said sample being contacted with a labeled specific binding member. In such an example, a solution containing the labeled specific binding member can include, or may not include, one or more dispersants of the present disclosure. Preferably, the solution contains the one or more dispersants to prevent dye-dye aggregation. However, it is within the scope of this disclosure that particular labeled specific binding members may not exhibit substantial aggregation, and in such circumstances the dispersants of the present disclosure may primarily be used to reduce non-specific binding of labeled specific binding members to cells.

Accordingly, in embodiments a method comprises reducing non-specific binding of a labeled specific binding member to a cell by contacting the cell with one or more dispersants (e.g., as herein described) prior to, or substantially concurrent with, contacting of the cell with the labeled specific binding member.

In embodiments, reducing non-specific binding refers to reducing non-specific binding fully (e.g., 100% reduction), or somewhat less than fully, for example a 99% reduction or more, or a 95% reduction or more, or a 90% reduction or more, or a 85% reduction or more, or a 80% reduction or more, or a 75% reduction or more, or a 70% reduction or more, or a 65% reduction or more or a 60% reduction or more, or a 55% reduction or more, or a 50% reduction or more, or a 45% reduction or more, as compared to a control sample where the cell is not contacted with any dispersant (e.g., as herein described).

### 11. Labeled Specific Binding Members

In embodiments, the labeled specific binding members of the present disclosure comprise fluorophores (e.g., polymeric tandem dye) conjugated to any variety of biomolecules, including proteins (e.g., antibodies), nucleic acids, affinity ligands, sugars, lipids, peptides, and substrates for enzymes. These formats are applicable to a wide variety of applications such as DNA microarrays, FISH assays, PCR assays, and also include the protein-based detection applications (e.g., FACS analysis) described above. Particularly advantageous in light of the present disclosure are properties of polymer materials used in polymeric tandem dyes that further allow for the amplification of more than one dye using a single excitation wavelength (laser, filter, etc). This enables simultaneous detection of multiple targets (multiplexing). Further details relating to multichromophores and their uses are disclosed in the following, each of which is incorporated herein by reference: U.S. patent application Ser. No. 11/329,495, filed Jan. 10, 2006, published as US 2006-0183140 A1; U.S. patent application Ser. No. 11/329,861, filed Jan. 10, 2006, published as US 2006-0216734 A1; U.S. patent application Ser. No. 11/344,942, filed Jan. 31, 2006, published as US 2006-0204984 A1; U.S. patent application Ser. No. 10/648,945, filed Aug. 26, 2003, published as US 2004-0142344 A1; U.S. patent application Ser. No. 10/600,286, filed Jun. 20, 2003, published as US 2004-0219556 A1; U.S. patent application Ser. No. 10/666,333, filed Sep. 17, 2003, published as US 2005-0059168 A1; and U.S. patent application Ser. No. 10/779,412, filed Feb. 13, 2004, published as US 2005-0003386 A1.

### B. Related Uses

### 1. Prevention and Reversal of Microsphere Aggregation

Microspheres as herein described find use in a wide variety of biomedical applications. For example, magnetic microspheres can be used for a variety of bioseparation applications. Specifically, magnetic particles can be coated with antibodies, lectins, oligonucleotides, and the like, to effect separations of specific cell populations and target sequences. As another example, microspheres can be used for a multitude of flow cytometry applications. For example, dyed microspheres (e.g., internally or externally) can be used in conjunction with flow cytometry instrument set-up, instrument quality control, compensation control, fluorescence quantitation, small particle size characterization, cell counting, and fluorescence reference quantitation. As yet another example, microspheres can be used for labeling of cells for imaging studies.

Microspheres useful for biomedical applications are in some examples made from polystyrene and other hydrophobic polymers, silica, and superparamagnetic polystyrene (or polystyrene infused with various amounts of iron oxide). Accordingly, regardless of the particular application, such microspheres are prone to aggregation, and certain applications can render microspheres increasingly prone to aggregation. For example, microspheres can be labeled on their external surface, and this labeling can increase aggregation susceptibility. Particularly relevant to the present disclosure include microspheres labeled with fluorophores, antibodies, and the like. One particular example of microspheres as herein described comprises quantum dots. Quantum dots as discussed herein and uses thereof may include any set forth in U.S. Pat. Nos. 10,717,927; 10,611,958; 9,804,319; 8,071,361; 6,207,392; 6,727,065; 7,311,774; 7,851,338; 6,501,091; 6,576,291; 7,125,605; 7,374,824; and 8,481,112. The entire contents of these patents are herein incorporated by reference in their entirety. Other examples of microspheres and uses thereof are provided in the following references, the contents of which are incorporated herein in their entirety, Joubert, M, et al., (2012) J Biol Chem 287:25266; Noyhouzer, T., et al., (2018) Journal of The Electrochemical Society, 165(2), H10-H15; Fernandez-Bravo A, et al., (2018) Nature Nanotech; 06/18: 1748-3395; Arifin, N, et al., (2018) Am J Trop Med Hyg, 98(4): 1165-1170; Choy C H, & Botelho R J. (2017) In Phagocytosis and Phagosomes (pp. 43-53); Gigault C, et al., (2001) Journal of colloid and interface science, 243(1), 143-155; Park, M K, et al., (2000) Clin Vaccine Immunol 7(3): 486-48; Blanchette C, et al., (2012) PLoS ONE 7(8): e42116; Rodrigues O R, & Monard S, (2016) Cytometry 89: 594-600; Borque L, et al., (2000) Clin Chem 46(11): 1839-1842; Perez-Amodio S, Holownia P, Davey C, Price C (2001) Anal. Chem. 73 (14): 3417-3425; Lucas L, Han J, Yoon J (2006) Colloids and Surf. B: Biointerfaces 49(2): 106-111; Fu L, et al., (2015) Anal Methods Issue 7; Deegan O, et al., (2003) Anal. Biochem. 312: 175-181; Koskinen J M, et al., (2018) Journal of immunological methods, 460: 113-118; Nanavaty N, et al., (2017) In Tau Protein (pp. 101-111). Humana Press, New York, NY; Schleicher K D, et al., (2014) Nat Nano 9, 525-530; Bandura D R, et al., (2009) Anal Chem 81, 6813-6822; Henderson, K., & Stewart, J. (2002) Journal of immunological methods, 270(1), 77-84; Henderson, K., & Stewart, J. (2000) Reproduction, Fertility and Development, 12(4), 183-189; Campbell, K., et al., (2007) Journal of agricultural and food chemistry, 55(6): 2497-2503; Terao, Y, et al., (2015) Journ of food protection, 78(8): 1560-1568; Sheng, W, et al., (2017) Microchimica Acta, 184(11), 4313-4321; Li S, et al., (2019) Frontiers in Cellular and Infection Microbiology, 9: 78; Kahanovitz L, et al., (2016) Journal of diabetes science and technology, 10(3): 689-696; Sepp, A, et al., (2002) FEBS Letters 532(3): 455-458; Kahanovitz L, et al., (2016) J Diabetes Sci Technol 10(3): 689-96; Tanga M Y H, Shum H C (2016) Lab Chip, 16: 4359-4365; Liang R-L, et al., (2015) Anal Chim Acta; 891: 277-283; Soukka T, et al., (2001) Clin Chem; 47(4): 1269-1278; Xia X, et al., (2013) Anal Bioanal Chem; 405(23): 7541-4; Yeo S J, et al., (2017) Scientific Reports, 7: 7933; Tuan Bao D, et al., (2017) Theranostics Vol 7, Issue 7: 1835-1846; Tang Y, et al., (2017) J Food Sci. March; 82(3):694-697; Do Thi Hoang Kim D T, et al., (2018) Theranostics, 8(13), 3629; Yu, S T, et al., (2018) Scientific reports, 8(1), 13468; Lee, M, et al., (2007) Nat. Protocols V.2 p 3226-3238; Mattheyses, A, et al., (2010) J Cell Sci 123: 3621-3628; Sun, Z, et al., (2005) Am J Physiol Heart Circ Physiol, 289(6) H2526-H2535; Ogilvie, J, et al., (2006) Optics Letters, Optical Society of America, 31(4), p 480-2; Chen X, et al., (2013) Review of Scientific Instruments 84,063701; Yang Z, et al., (2017) IEEE Journal of Selected Topics in Quantum Electronics, 23(3), 59-63; Adams E W, et al., (2003) Angew Chem Int Ed Engl. 10; 42(43):5317-20; Shapiro, et al., (2010) Water Research, 44(3), 893-903; Pei X, (2017) Anal. Chem. 10.1021/acs.analchem.7b04551; Hinds, K, et al., (2003) Blood August 102 (3) 867-872; Shapiro, E M, et al., (2005) Magn. Reson. Med., 53: 329-338; Rohani, R, et al., (2011) Mol Imaging Biol 13: 679; Slotkin, J, et al., (2007) Neurotherapeutics, V4,3, P428-433; Tarulli E, et al., (2013) J Magn Reson Imaging. June; 37(6):1409-18; Campbell, K., et al., (2007) Journal of agricultural and food chemistry, 55(6), 2497-2503; Terao, Y, et al., (2015) Journ of food protection, 78(8), 1560-1568; Sheng, W, et al., (2017) Microchimica Acta, 184(11), 4313-4321; Li S, et al., (2019) Frontiers in Cellular and Infection Microbiology, 9: 78; Ferguson J A, et al., (2000) Anal Chem; 72(22):5618-5624; Lauer S, et al., (2002) Biochemistry; 41(6):1742-51; Steinberg G, et al., (2004) Biopolymers; 73(5):597-605; B Sharma, et al., (2016) Am Chem Soc, 138 (42), pp 13952-13959; G Steinberg-Tatman, M et al., (2006) Chem 17(3), 841-848; N Clack, et al., (2008) Nat Biotech 26,825-830; Lu Q, et al., (2013) Stem Cells Dev. V22:18; Khan Z, et al., (2008) Anal Biochem. April 15; 375(2):391-3; Marrec P, et al., (2017) Biogeosciences Discuss, https://doi.org/10.5194/bg-2017-343; Wang, Z. (2016) Nanoscience, 3, 193-210; Crites, T. J., et al., (2015) Current protocols in cell biology, 24-5; Zanoni, M., et al., (2016) Biotechnology and bioengineering, 113(3), 501-512; Tolić-Nørrelykke, et al., (2006) Review of scientific instruments, 77(10), 103101; Bhebhe N, et al., (2018) Sci. Rep. Vol. 8: 17387; Wang L, et al., (2004) Nano Lett; 5(1):37-43; Lauer, SA, Nolan, JP (2002) Cytometry, 48: 136-145; Yoo C E, et al., (2015) Biomaterials January; 75:271-8; Litvinov, J, et al., (2016) Biomicrofluidics, 10(1), 014103; Litvinov J, et al., (2017) Journal of Bioengineering & Biomedical Science, 6(SAND-2015-11159J); Iswardy E, et al., (2017) Biosensors and Bioelectronics, 95, 174-180; Nandin I S, et al., (2017) J Exp Med. 20170633; Wu S J, et al., (2004) J Microbiol Methods Vol 56, Issue 3, 395-400; Walter S, et al., (2003) J Immunol. 171(10)4974-4978; Jeffers, Faye, et al., Carbohydrate research 345.10 (2010): 1486-1491; Pitsillides, et al., (2003) Biophysical journal, 84(6), 4023-4032; Linz T H, et al., (2017) Lab on a Chip, 17(6), 1076-1082; Chang D, et al., (2017) 7(1), 3110; Bhattacharyya, K., et al., (2016) Journal of biomedical optics, 21(8), 087007; Wang Y, et al., (2018) Frontiers in Microbiology, 9; Blumberg, S, et al., (2005) Biophysical Journal V89(2) p 1272-1281; Wang J, et al., (2001) Anal Chem. 73(22), 5576-5581; Reynolds N M, et al., (2018) Cell Mol Bioeng; 11(1):37-52; Fisher S, et al. (2011) Genome Biol; 12(1), R1; Elkin C J, et al. (2001) Genome Res; 11(7), 1269-1274; Vemulapati S, & Erickson D. (2018) Lab on a Chip 18(21), 3285-3292; McKeague, M, et al., (2010) Int. J. Mol. Sci, 11(12), 4864-4881; Pryor, P, Rofe, A (2014) Cold Spring Harb Protoc; doi:10.1101/pdb.prot074468; Agidi S, et al., (2013) J Environ Manage. January 30; 115:167-74; Scida, K, et al., (2014) Anal Chem 86(13) 6501-6507; Srbova, J, et al., (2018) Electrophoresis, 39(3), 526-533; Ouk M, & Beach G S. (2017) Journal of Magnetism and Magnetic Materials, 444, 218-226; Zhou, M., et al., (2019) ACS Omega, 4(4), 6931-6938; Diekmann, R., et al., (2016) Nature Communications, 7; Hill, J, et al., (2003) Circulation. 108:1009-1014; Shapiro E M, et al., (2006) Magn. Reson. Med., 55: 242-249; Shapiro, E M, et al., (2007) Contrast Media Mol Imaging, 2: 147-153; Shapiro, E M, et al., (2006) NeuroImage, 32(3), 1150-1157; Tomin A, et al., (2016) Mat-wiss u. Werkstofftech, 47: 189-192; Thuy Tram L L, et al., (2012) Food Control, Vol 24, Issues 1-2, 23-28; Sun, Liangliang, et al. Journal of Chromatography A 1220 (2012): 68-74; Sroka-Bartnicka A, et al., (2017) J Pharm Biomed Anal, V. 132 p 125-132; Barnard, G. C., et al., (2010) J Ind Microbiol Biotechnol Vol 37(9) pp 961-971; Yang, Ganglong, et al. Proteomics 13.9 (2013): 1481-1498; Skene, Peter J., and Steven Henikoff. bioRxiv (2017): 193219; Kaminski R W, et al., (2014) Clin Vaccine Immunol; 21(3):366-382; Cadena-Herrera D, et al., (2015) Biotechnol Reports; 7:9-16; Lew C, et al., (2012) Instrument-to-instrument Variability in the Vi-CELL Automated Viability Analyzer. Beckman Coulter Life Sciences; Chen A, et al., (2017) PLoS One; 12(3):e0173375; Pagliuca F W, et al., (2014) Cell, 159(2), 428-439; Estes, Myka L., et al. Current protocols in cytometry (2010): 9-33; Purvis N, Stelzer G. (1998) Cytometry; 33(2):156-65; Miao, Q, et al., (2009) Proc. SPIE 7262, Medical Imaging: Biomedical Applications in Molecular, Structural, and Functional Imaging; Choi H, et al., (2012) Proc. SPIE 8548, Nanosystems in Engineering and Medicine, 85484A; Mao X, et al., (2009) Lab Chip, 2009, 9, 1583-1589; Meineke, G, et al., (2013) Proc. SPIE 8615, Microfluidics, BioMEMS, and Medical Microsystems XI; Jillavenkatesa, A, et al., (2001) Natl. Inst. Stand. Technol. Spec. Publ. 960-1; Hassellov, M, et al., (2008) Ecotoxicology (2008) 17:344-361; Vogel, R, et al., (2012) Anal. Chem 84(7, p 3125-3131; Vogel R, et al., (2017) Scientific reports, 7(1), 17479; Philips L A, et al., (2017) Water research, 122, 431-439; Proulx K, et al., (2016) Anal & Bioanal Chem, 408(19), 5147-5155; Mizutani, Y, et al., (2015) MATEC Conf. 32, 05001; Musil, J, et al., (2014) Cancer gene therapy, 21(3), 115; Wang J-C E, et al., (2009) J Immunol Methods; 341:106-116; Scheible K, et al., (2012) Cytometry A 81(11):937-949; Perfetto S P, et al., (2006) J Immunol Methods; 313(1-2):199-208; Perfetto S P, et al., (2010) Curr Protoco Cytom; July; CHAPTER: Unit-9.34; Lamoreaux, L, et al., (2006) Nat. Protocols 1, 1507-1516; Lugli E, et al., (2012) Nature Protocol v8(1); Estes, M. L et al., (2010) Current protocols in cytometry, 9-33; Wyant T, et al., (2008) J Transl Med; 6:76; Quadrini K J, et al., (2016) Cytometry B Clin Cytom; 90B:177-190; Bray R A. (2013) Methods Mol Biol; 1034:285-96; Kay S, et al., (2006) Cytometry, 70B: 218-226; Muller L, et al., (2016) Sci Rep 6:20254; Singh H, et al., (2013) PLoS ONE; 8(5):e64138; Homann S, et al., (2017) PloS one, 12(9), e0182941; Bringeland, G. H., et al., (2019) 95(3): 314-322; Randlev B, et al., (2010) Biologicals; 38(2):249-259; McBean R S, et al., (2015) Blood Transfus; 13:662-5; Phillips A, et al., (2016) Mol Cancer Ther 15(4)661-669; Tillotson B, et al., (2013) Methods 60(1)27-37; Andreev J, et al., (2017) Mol Cancer Ther. (16)(4) 681-693; Kong, F, et al., (2015) Experimental and Therapeutic Medicine, 10, 2093-2101; Huang S, et al., Lab on a chip. 2015; 15(2):448-458; Ostergaard E, et al., (2015) Biologicals Vol. 43, 4:266-273; Venable A S, et al., (2014) J Immunol Methods, Vol 406, 117-123; Fischer S, et al., (2016) PLoS One, 11(9), e0163665; Mathew G, et al., (2009) Cell Cycle, 8:7, 1053-1061; Obokata H, et al., (2011) Tissue Eng Part A 17(5-6):607-15; Dosen G, et al., (2006) BMC Immunol. June 29; 7:13; Xiong, Z, et al., (2012) Vox Sang 103(1) p 42-48; Borzova, E, Dahinden, C, (2014) Meth Mol Biol V. 1192 p 87-100; Moss D M, et al., (2004) J Parasitol; 90(2):397-404; Yu J, et al., (2008) J Med Microbiol; 57(Pt 2):171-8; Kellar, KL, Iannone, M (2002) Exp. Hematol, V30, I1, p 1227-1237; Hale M, et al., (2017) Mol Ther Methods Clin Dev; Vol 4 p 192-203; Kellar K L, et al., (2006) Curr Protoc Cytom. February; Chapter 13: Unit 13.1; Dolgushin S A, et al., (2015) Biomed Eng. Vol 49, 2, 85-89; Spiro A, et al., (2000) Appl. Environ. Microbiol. Vol 66(10) p 4258-4265; Yang S, et al., (2008) Biosens. Bioelectron Vol 24:4 855-862; Joslin J, et al., (2018) 05/29 SLAS DISC: Adv Life Sci R&D; Cretich, M., et al., (2009) Proteomics, 9(8), 2098-2107; Franklin, R B, et al., (2011) Journal of microbiological methods, 87(2), 154-160.

Factors that additionally contribute to microsphere aggregation include but are not limited to microsphere size (e.g., aggregation likelihood decreases as diameter increases), surface charge (e.g., the greater the surface charge, the lower the likelihood of aggregation), temperature (e.g., higher temperatures increase kinetics of microspheres in suspension, hence increasing likelihood of hydrophobic contact), and concentration (e.g., increased concentration increases the likelihood of hydrophobic interactions), among others.

The dispersants of the present disclosure are herein recognized as particularly advantageous for use as agents capable to substantially reduce, or even prevent entirely, aggregation of microspheres. In addition to being effective at reducing or avoiding aggregation, the dispersants of the present disclosure exhibit no absorption or emission characteristics within the range of 350-850 nm, exhibit no adverse effects on biomolecules (e.g., do not denature proteins, do not interfere with normal biological process such as binding between a specific binding member and a target analyte, etc.), and can be readily used with various instrumentation systems (e.g., FACS systems).

Current recommendations for avoiding microsphere aggregation center on physical approaches including but not limited to sonication, vortexing, pipetting, or some combination of the above. Yet there are disadvantages to such approaches. First, such approaches are confined to specific periods of time, and depending on the application, aggregation may still occur subsequent to the initial de-aggregation procedure. Second, sonication comes with the potential of contamination of the microsphere suspension, shedding of metal, and the like. Bath sonication can be an alternative, yet even then the introduction of heat to the system can have adverse effects on microsphere properties depending on the application. In terms of vortexing, shear forces involved can in examples dislodge passively adsorbed ligand, and similar issues are observed with pipetting approaches.

Similar to that discussed above, the dispersants of the present disclosure are capable to effectively prevent or reverse microsphere aggregation. The prevention or reversal of microsphere aggregation can be realized in absence of other physical methodologies, such as sonication, vortexing, pipetting, and the like. However, it is within the scope of this disclosure that some amount of combined approaches can be used, such as prevention or reversal of microsphere aggregation via one or more dispersants and one or more physical approaches mentioned above. When physical approaches are used, the amount of energy input to the microsphere solution may be lesser than situations where a microsphere solution lacks the one or more dispersants as herein described. For example, a level and/or duration of sonication may be reduced (e.g., by 50% or more, such as 80% or 90% or more), a speed and/or duration of vortexing may be substantially reduced (e.g., by 50% or more, such as 80% or 90% or more), and/or a speed and/or duration at which pipetting is conducted may be substantially reduced (e.g., by 50% or more, such as 80% or 90% or more).

### 2. Use as Wetting Agents

The polymers of the present disclosure substituted with at least one or more aromatic side groups connected via a saturated hydrocarbon chain are also herein recognized as remarkably advantageous for use as wetting agents, particularly when used as wetting agents in procedures that include a biological sample, for example a biological sample that comprises one or more cells. Specifically, when used as wetting agents, the polymers as herein disclosed can significantly reduce an amount of biological material (e.g., cells, proteins, peptides, nucleic acids, and the like) that otherwise may adhere to, for example, labware, in the absence of said polymers. Accordingly, use of such polymers as wetting agents can reduce an amount of sample lost during experimental procedures.

This is illustrated at Example 4 below, where it is demonstrated that cell recovery after multiple wash steps, analyzed by FACS, is substantially improved, even under circumstances where the cells are free from any labeled specific binding member (unstained).

Advantages to the use of the polymers of the present disclosure as wetting agents include but are not limited to circumstances where the biological sample is challenging to obtain in high yield (e.g., tumor cells), and where a particular assay requires a particular amount of the biological sample for robust analysis.

In embodiments, recovery of a biological material after one or more procedural steps (e.g., wash steps) may be increased by about 2-fold, about 3-fold, about 4-fold, about 5-fold, about 6-fold, about 7-fold, about 8-fold, about 9-fold, about 10-fold, between 10-fold and 20-fold, or even greater than 20-fold, as compared to a similar procedure conducted in absence of the wetting agent. For the increased recovery, the wetting agents may be included at percent w/v of between 0.001% and 15%, in embodiments. In additional or alternative embodiments, the wetting agents may be included at concentrations of between about 0.001 μg/mL and about 100 mg/mL.

### III. Systems

Aspects of the invention further include systems for use in practicing the subject methods and compositions. A sample analysis system may include a flow channel loaded with a sample and a labelled specific binding member. In some embodiments, the system is a flow cytometric system including: a flow cytometer including a flow path; a composition in the flow path, wherein the composition includes: a sample; a dispersant and a labelled specific binding member (e.g., as described herein).

In certain embodiments of the system, the composition further includes a second specific binding member that is support bound and specifically binds the target analyte. In some cases, the support includes a magnetic particle. As such, in certain instances, the system may also include a controllable external paramagnetic field configured for application to an assay region of the flow channel.

The sample may include a cell. In some instances, the sample is a cell-containing biological sample. In some instances, the sample includes a labelled specific binding member specifically bound to a target cell. In certain instances, the target analyte that is specifically bound by the specific binding member is a cell surface marker of the cell. In certain cases, the cell surface marker is selected from a cell receptor and a cell surface antigen.

In certain aspects, the system may also include a light source configured to direct light to an assay region of the flow channel. The system may include a detector configured to receive a signal from an assay region of the flow channel, wherein the signal is provided by the fluorescent composition. Optionally further, the sample analysis system may include one or more additional detectors and/or light sources for the detection of one or more additional signals.

In certain aspects, the system may further include computer-based systems configured to detect the presence of the fluorescent signal. A “computer-based system” refers to the hardware means, software means, and data storage means used to analyze the information of the present invention. The minimum hardware of the computer-based systems of the present invention includes a central processing unit (CPU), input means, output means, and data storage means. A skilled artisan can readily appreciate that any one of the currently available computer-based system are suitable for use in the subject systems. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming and can be read by a suitable reader communicating with each processor at its corresponding station.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In certain aspects, the system includes a flow cytometer. Flow cytometers of interest include, but are not limited, to those devices described in U.S. Pat. Nos. 4,704,891; 4,727,029; 4,745,285; 4,867,908; 5,342,790; 5,620,842; 5,627,037; 5,701,012; 5,895,922; and 6,287,791; the disclosures of which are herein incorporated by reference.

Other systems may find use in practicing the subject methods. In certain aspects, the system may be a fluorimeter or microscope loaded with a sample having a fluorescent composition of any of the embodiments discussed herein. The fluorimeter or microscope may include a light source configured to direct light to the assay region of the flow channel. The fluorimeter or microscope may also include a detector configured to receive a signal from an assay region of the flow channel, wherein the signal is provided by the fluorescent composition.

### IV. Kits

Aspects of the invention further include kits for use in practicing the subject methods and compositions. The compositions of the invention can be included as reagents in kits either as starting materials or provided for use in, for example, the methodologies described above.

A kit may include one or more of a labeled specific binding member, one or more dispersants and/or wetting agents (e.g., as herein described), a cell, biocompatible buffer(s), and instructions for use. In embodiments, the labeled specific binding member comprises one of a tandem dye, a polymeric tandem dye, a small organic dye (e.g., fluorescein), a fluorescent protein (e.g., GFP, YFP, and the like), etc. The kit may include instructions for use.

In certain embodiments, the kit finds use in evaluating a sample for the presence of a target analyte, such as an intracellular target. As such, in some instances, the kit includes one or more components suitable for lysing cells. The one or more additional components of the kit may be provided in separate containers (e.g., separate tubes, bottles, or wells in a multi-well strip or plate).

In certain embodiments, the kit may additionally or alternatively include at least one or more (e.g., a variety of) microspheres which can be coated with various functional agents (e.g., fluorophores, antibodies, and the like), and one or more dispersants as herein described. The microspheres and the one or more dispersants may in some examples be combined together in the kit, to prevent microsphere aggregation during storage thereof. In additional or alternative examples, the microspheres and the one or more dispersants may be included in separate containers, with instructions on how the one or more dispersants may be used to reverse aggregation that may occur during storage.

In certain aspects, a kit further can include reagents for performing a flow cytometric assay. Reagents of interest include, but are not limited to, buffers for reconstitution and dilution, buffers for contacting a cell sample with the labeled specific binding member, wash buffers, control cells, control beads (e.g., microspheres as discussed above), fluorescent beads (e.g., microspheres as discussed above) for flow cytometer calibration and combinations thereof. The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The compositions of the kit may be provided in a liquid composition, such as any suitable buffer. Alternatively, the compositions of the kit may be provided in a dry composition (e.g., may be lyophilized, or vitrified), and the kit may optionally include one or more buffers for reconstituting the dry composition. In certain aspects, the kit may include aliquots of the compositions provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### V. Utility

The compositions, methods, and systems as described herein may find use in a variety of applications, including diagnostic and research applications, in which the labeling detection and/or analysis of a target of interest is desirable. Such applications include methodologies such as cytometry, microscopy, immunoassays (e.g., competitive or non-competitive), assessment of a free analyte, assessment of receptor bound ligand, and so forth. The compositions, system and methods described herein may be useful in analysis of any of a number of samples, including but not limited to, biological fluids, cell culture samples, and tissue samples. In certain aspects, the compositions, system and methods described herein may find use in methods where analytes are detected in a sample, if present, using fluorescent labels, such as in fluorescent activated cell sorting or analysis, immunoassays, immunostaining, and the like. In certain instances, the compositions and methods find use in applications where the evaluation of a sample for the presence of a target analyte is of interest.

In some cases, the methods and compositions find use in any assay format where the detection and/or analysis of a target from a sample is of interest, including but not limited to, flow cytometry, in-situ hybridization, enzyme-linked immunosorbent assays (ELISAs), western blot analysis, magnetic cell separation assays and fluorochrome purification chromatography. In certain instances, the methods and compositions find use in any application where the fluorescent labelling of a target molecule is of interest. The subject compositions may be adapted for use in any convenient applications where pairs of specific binding members find use, such as biotin-streptavidin and hapten-anti-hapten antibody.

### Additional Embodiments

In some embodiments, set forth is a compound having the following structure:

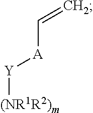

wherein: A is a six-membered aryl or a six-membered heteroaryl, wherein heteroaryl comprises one to three heteroatoms selected from the group consisting of N, O, S, and combinations thereof, Y is, individually in each instance, C═O or SO₂; wherein R¹ and R² are each, individually in each instance, C₁₋₆alkyl or [PEG]ₚ-R^{A}; R^{A} is H or C₁₋₆alkyl; subscript m is an integer of 1 to 3; and subscript p is an integer of 1 to 10.

In some embodiments, including any of the foregoing, the compound has the following structure:

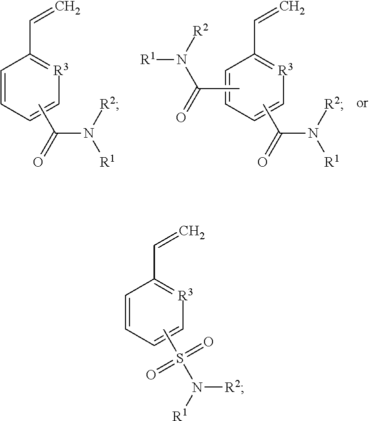

wherein R¹ and R² are each, individually in each instance, methyl or —C₂H₂O—[C₂H₂O]ₙ—R⁴; R³ is CH or N; R⁴ is H or methyl; and wherein subscript n is 0, 1, 2, 3, 4, or 5. In certain embodiments, the compound has the following structure:

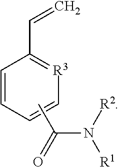

In certain other embodiments, the compound has the following structure:

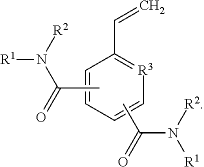

In yet other embodiments, the compound has the following structure:

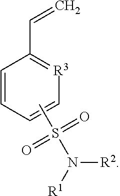

In some embodiments, including any of the foregoing, the compound has the following structure:

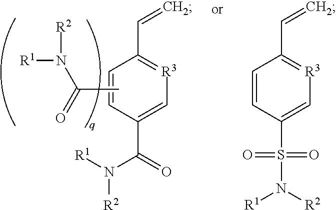

wherein subscript q is 0, 1, or 2. In certain embodiments, the compound has the following structure:

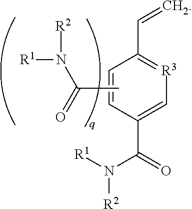

In certain other embodiments, the compound has the following structure:

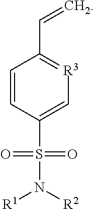

In some embodiments, including any of the foregoing, subscript q is 0.

In some embodiments, including any of the foregoing, subscript q is 1.

In some embodiments, including any of the foregoing, subscript n is 0.

In some embodiments, including any of the foregoing, R⁴ is H.

In some embodiments, including any of the foregoing, the compound has the following structure:

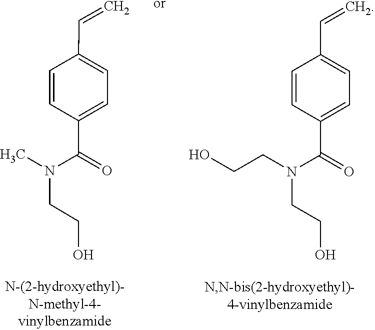 [SMILES: C=Cc1ccc(C(=O)N(C)CCO)cc1 ; "or" ; C=Cc1ccc(C(=O)N(CCO)CCO)cc1]

In some embodiments, including any of the foregoing, the compound has the following structure:

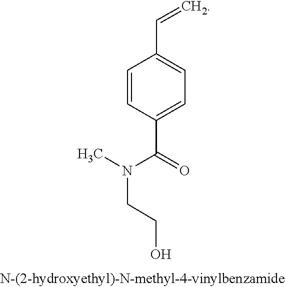

In some embodiments, including any of the foregoing, the compound has the following structure:

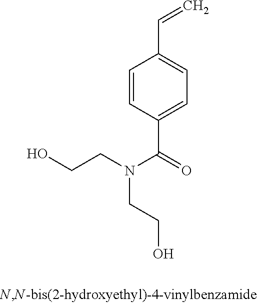 [SMILES: C=Cc1ccc(C(=O)N(CCO)CCO)cc1]

In some embodiments, including any of the foregoing, the compound has the following structure:

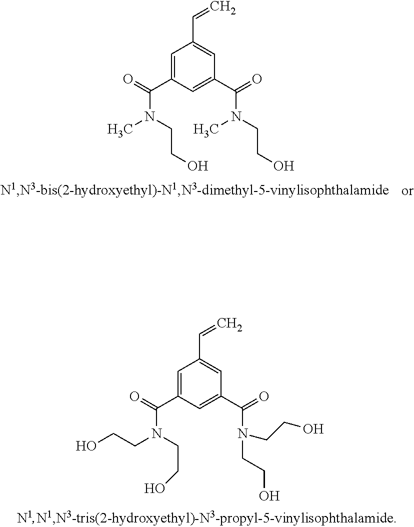 [SMILES: C=Cc1cc(C(=O)N(C)CCO)cc(C(=O)N(C)CCO)c1 ; C=Cc1cc(C(=O)N(CCO)CCO)cc(C(=O)N(CCO)CCO)c1]

In some embodiments, including any of the foregoing, the compound has the following structure:

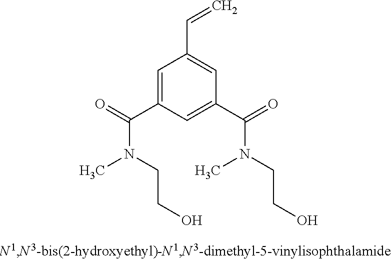 [SMILES: C=Cc1cc(C(=O)N(C)CCO)cc(C(=O)N(C)CCO)c1]

In some embodiments, including any of the foregoing, the compound has the following structure:

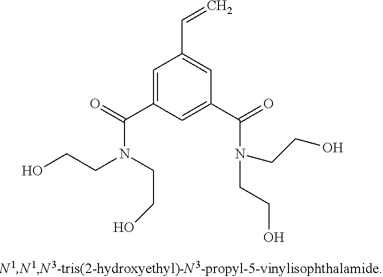 [SMILES: C=Cc1cc(C(=O)N(CCO)CCO)cc(C(=O)N(CCO)CCO)c1]

In some embodiments, set forth is a polymer made by polymerizing a solution or mixture comprising at least one compound set forth herein.

In some embodiments, including any of the foregoing, the polymer is water soluble.

In some embodiments, including any of the foregoing, the polymer is transparent to 350 nm to 800 nm wavelength light.

In some embodiments, including any of the foregoing, the polymer has an average molecular weight that is less than 10,000 Da.

In some embodiments, including any of the foregoing, the polymer has an average molecular weight of 1,000 to 5,000 Da.

In some embodiments, including any of the foregoing, the polymer has a dispersity less than 3 or less than 2.

In some embodiments, set forth is a polymer comprising a saturated hydrocarbon substituted with at least one or more substituents, wherein each substituent, individually in each instance is: (a) selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; heteroaryl-heteroalkyl; and combinations thereof, and (b) non-ionic at 6≤pH≤8; (c) optionally substituted with 1 to 6 substituents selected from the group consisting of polyethylene glycol (PEG), —OH, —SH, —NO₂, —NO₃, —C(O)OH, —NH₃, —CH₃, —CH₂—OH, —CH₂—CH₂—OH, ═O, and —CN, wherein the optional substituents are not further substituted; wherein the polymer: (c) is water soluble; and (d) is transparent to 350 nm to 800 nm wavelength light.

In some embodiments, including any of the foregoing, the at least one or more substituents constitute 1% to 100% of the substituents present on the polymer.

In some embodiments, including any of the foregoing, the at least one or more substituents constitute 1% to 50% of the substituents present on the polymer.

In some embodiments, including any of the foregoing, the at least one or more substituents constitute 1% to 20% of the substituents present on the polymer.

In some embodiments, including any of the foregoing, the at least one or more substituents constitute 5%-20% w/w of the polymer.

In some embodiments, including any of the foregoing, the polymer is non-ionic at 6≤pH≤8.

In some embodiments, including any of the foregoing, the at least one or more substituents comprises two or more substituents.

In some embodiments, including any of the foregoing, the at least one or more substituents comprises five or more substituents.

In some embodiments, including any of the foregoing, the at least one or more substituents are substituted with PEG groups.

In some embodiments, including any of the foregoing, the PEG groups comprise PEG₄.

In some embodiments, including any of the foregoing, the polymer has an aqueous solubility of ≥1 mg/mL.

In some embodiments, including any of the foregoing, the polymer has an aqueous solubility of ≥10 mg/mL.

In some embodiments, including any of the foregoing, the polymer has an aqueous solubility of ≥100 mg/mL.

In some embodiments, including any of the foregoing, the polymer has average molecular weight (MW) between 500-5,000,000 Daltons.

In some embodiments, including any of the foregoing, the polymer has an average MW between 1000-100,000 Daltons.

In some embodiments, including any of the foregoing, the polymer has an average MW between 1000-10,000 Daltons.

In some embodiments, including any of the foregoing, the polymer comprises poly(vinyl alcohol).

In some embodiments, including any of the foregoing, at least one or more substitutes are selected from alkyl-aryl or aryl-alkyl.

In some embodiments, including any of the foregoing, the polymer is PEG₄ and the alkyl-aryl is benzaldehyde.

In some embodiments, including any of the foregoing, the polymer is made by a process that comprises polymerizing monomers having an aqueous solubility of ≥10 mg/mL.

In some embodiments, including any of the foregoing, the polymer is made by a process that comprises radical or ionic polymerization of vinyl monomers.

In some embodiments, including any of the foregoing, the polymer is made by a process that comprises post polymerization modification of polyvinyl alcohol (PVA).

In some embodiments, including any of the foregoing, the polymer is made by a process that comprises arylation of PVA by using acetalization of the at least one or more substituents as monomers.

In some embodiments, including any of the foregoing, the polymer is made by a process that comprises post polymerization modification of styrene-alt-maleic anhydride copolymer—SMA (50:50).

In some embodiments, including any of the foregoing, the polymer is made by a process that comprises aromatic monomer copolymerization with maleic anhydride and post polymerization modification of the maleic anhydride.

In some embodiments, including any of the foregoing, the polymer is made by a process that comprises post polymerization modification of methyl vinyl ether-alt-maleic anhydride copolymer (50:50).

In some embodiments, set forth is a polymer comprises a structure selected from Formula (I):

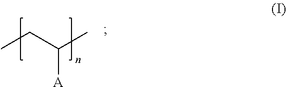

wherein A is, independently in each instance, selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; and heteroaryl-heteroalkyl; and subscript n is an integer from 1 to 100,000.

In some embodiments, including any of the foregoing, A is selected from:

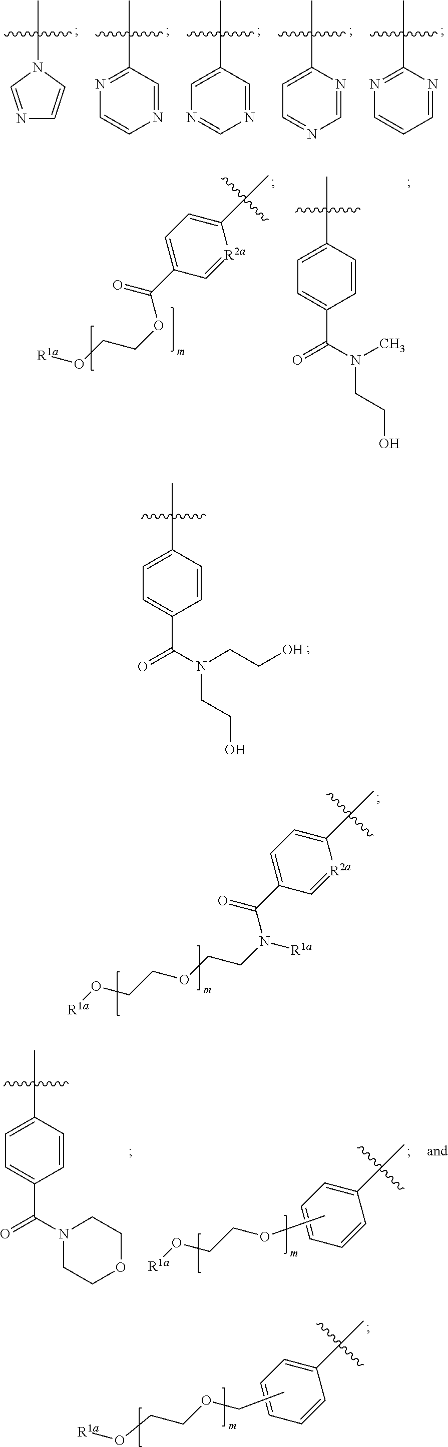

wherein subscript m is an integer from 1 to 10; R^{1a} is H, CH₃, or —C₂H₂O—[C₂H₂O]ₙ—R^{4a}; R^{2a} is CH or N; R^{4a} is H or methyl; and wherein subscript n is 0, 1, 2, 3, 4, or 5; and

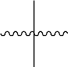

is the bond from A to Formula (I).

In some embodiments, including any of the foregoing, polymer comprises the structure:

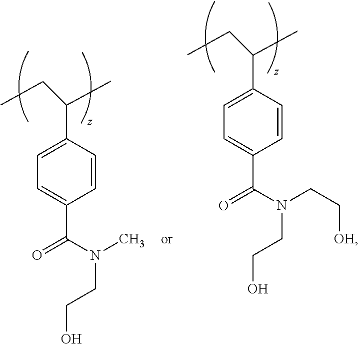

wherein z is an integer of 1 to 100,000.

In some embodiments, including any of the foregoing, polymer comprises the structure:

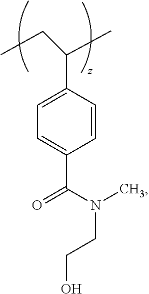

wherein z is an integer of 1 to 100,000.

In some embodiments, including any of the foregoing, polymer comprises the structure:

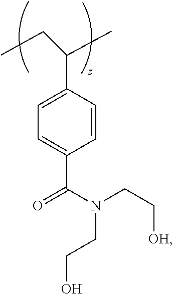

wherein z is an integer of 1 to 100,000.

In some embodiments, including any of the foregoing, polymer comprises the structure:

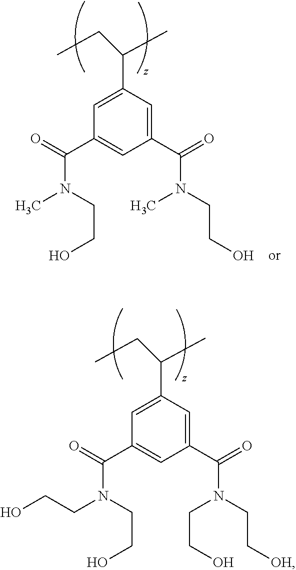

wherein z is an integer of 1 to 100,000.

In some embodiments, including any of the foregoing, polymer comprises the structure:

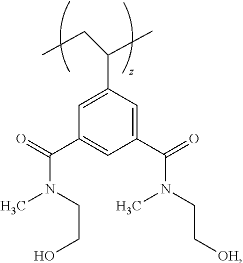

wherein z is an integer of 1 to 100,000.

In some embodiments, including any of the foregoing, polymer comprises the structure:

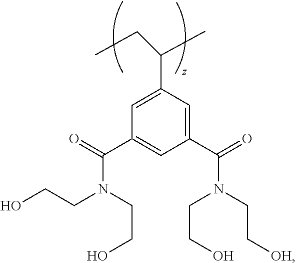

wherein z is an integer of 1 to 100,000.

In some embodiments, including any of the foregoing, the average molecular weight of the polymer is less than 10,000 Da.

In some embodiments, including any of the foregoing, the average molecular weight of the polymer is between 1,000-5,000 Da.

In some embodiments, including any of the foregoing, the polymer has a dispersity index less than 3.

In some embodiments, including any of the foregoing, the polymer has a dispersity index less than 2.

In some embodiments, set forth is a polymer that includes a structure selected from Formula (II):

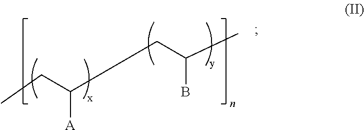

wherein A and B are each, independently in each instance, selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; heteroaryl-heteroalkyl; cycloalkyl; heteroalkyl; and heterocycloalkyl; and combinations thereof, wherein A is optionally substituted with one to three —NR¹R²; wherein heteroalkyl and heterocycloalkyl are optionally substituted with 1 to 6 substituents selected from the group consisting of polyethylene glycol (PEG), —OH, —SH, —NO₂, —NO₃, —C(O)OH, —NH₃, —CH₃, —CH₂—OH, —CH₂—CH₂—OH, ═O, and —CN, wherein the optional substituents are not further substituted; wherein R¹ and R² are each, individually in each instance, C₁₋₆alkyl or [PEG]ᵣ—R⁴; R⁴ is H or C₁₋₆alkyl; and subscript r is 1 to 10; subscript x is an integer from 1 to 50; subscript y is an integer from 1 to 50; and subscript n is an integer from 1 to 100,000.

In some embodiments, set forth is a polymer that includes a structure selected from Formula (IIa):

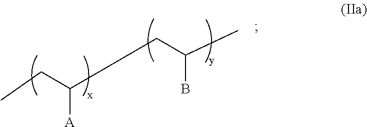

wherein A and B are each, independently in each instance, selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; heteroaryl-heteroalkyl; cycloalkyl; heteroalkyl; and heterocycloalkyl; and combinations thereof, wherein A is optionally substituted with one to three —NR¹R²; wherein heteroalkyl and heterocycloalkyl are optionally substituted with 1 to 6 substituents selected from the group consisting of polyethylene glycol (PEG), —OH, —SH, —NO₂, —NO₃, —C(O)OH, —NH₃, —CH₃, —CH₂—OH, —CH₂—CH₂—OH, ═O, and —CN, wherein the optional substituents are not further substituted; wherein R¹ and R² are each, individually in each instance, C₁₋₆alkyl or [PEG]ᵣ—R⁴; R⁴ is H or C₁₋₆alkyl; and subscript r is 1 to 10; and the ratio of subscript x to subscript y is [0.05 to 0.25]:[0.75 to 0.95].

In some embodiments, including any of the foregoing, the ratio of subscript x to subscript y is [0.15 to 0.2]:[0.8 to 0.85].

In some embodiments, including any of the foregoing, B is selected from the group consisting of:

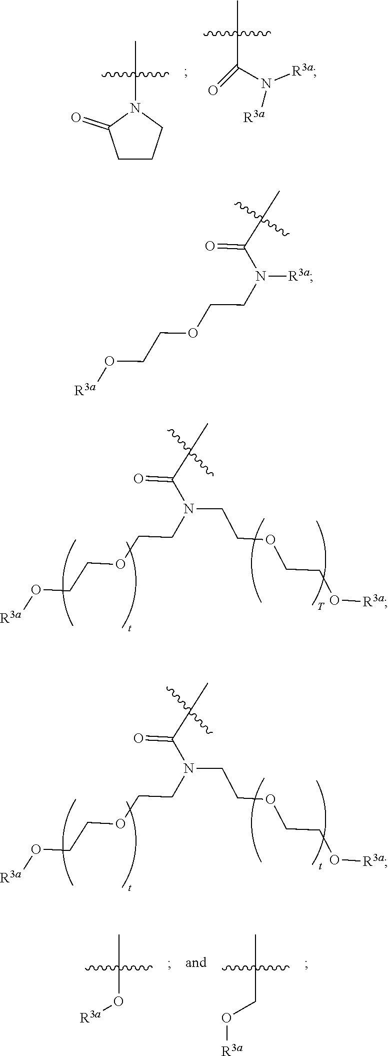

wherein subscript t is an integer from 0 to 10; wherein R^{3a} is H or CH₃; and

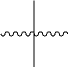

is the bond from B to Formula (II).

In some embodiments, including any of the foregoing, subscript t is an integer from 0 to 5.

In some embodiments, including any of the foregoing, subscript t is 0.

In some embodiments, including any of the foregoing, A is selected from:

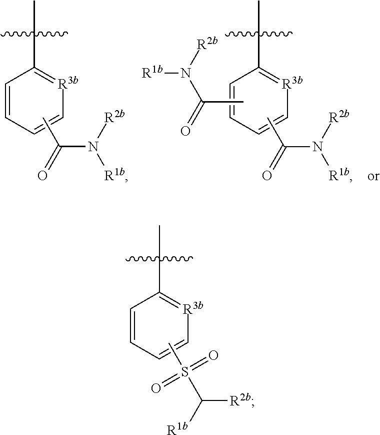

wherein R^{1b} and R^{2b} are each, individually in each instance, methyl or —C₂H₂O—[C₂H₂O]ᵤ—R^{4b}; R^{3b} is CH or N; R^{4b} is H or methyl; and wherein subscript n is 0, 1, 2, 3, 4, or 5.

In some embodiments, including any of the foregoing, A is selected from:

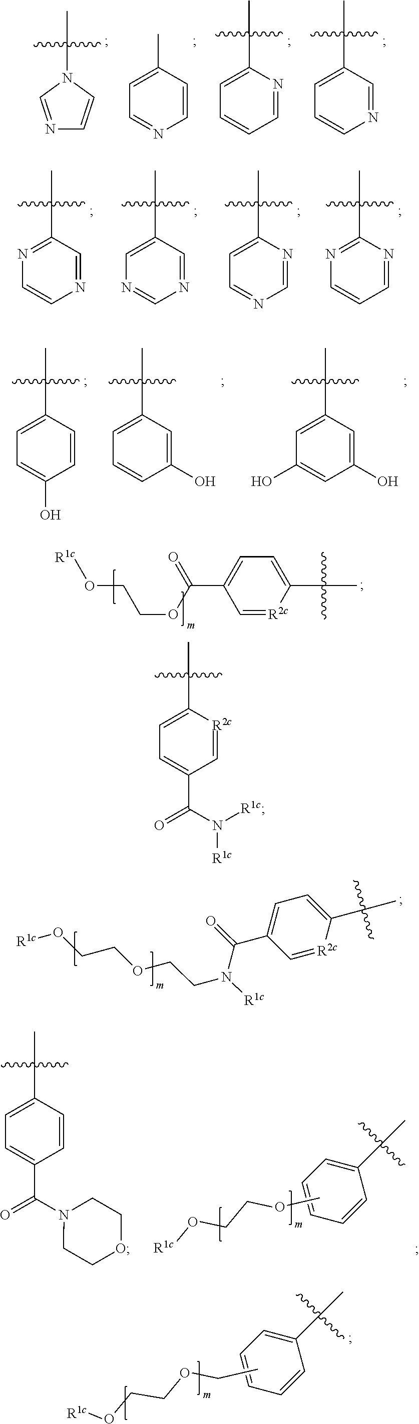

wherein subscript m is an integer from 1 to 10; R^{1c} is H or CH₃; R^{2c} is CH or N; and

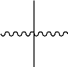

is the bond from A to Formula (IIa).

In some embodiments, including any of the foregoing, the polymer comprises a structure selected from

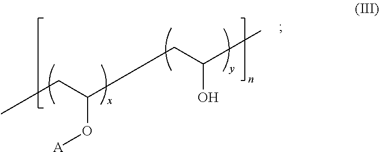

wherein A is, independently in each instance, selected from the group consisting of aryl, alkyl-aryl; heteroalkyl-aryl; heteroaryl, alkyl-heteroaryl, and heteroalkyl-heteroaryl; subscript x is an integer from 1 to 50; subscript y is an integer from 1 to 50; and subscript n is an integer from 1 to 100,000.

In some embodiments, including any of the foregoing, A is selected from:

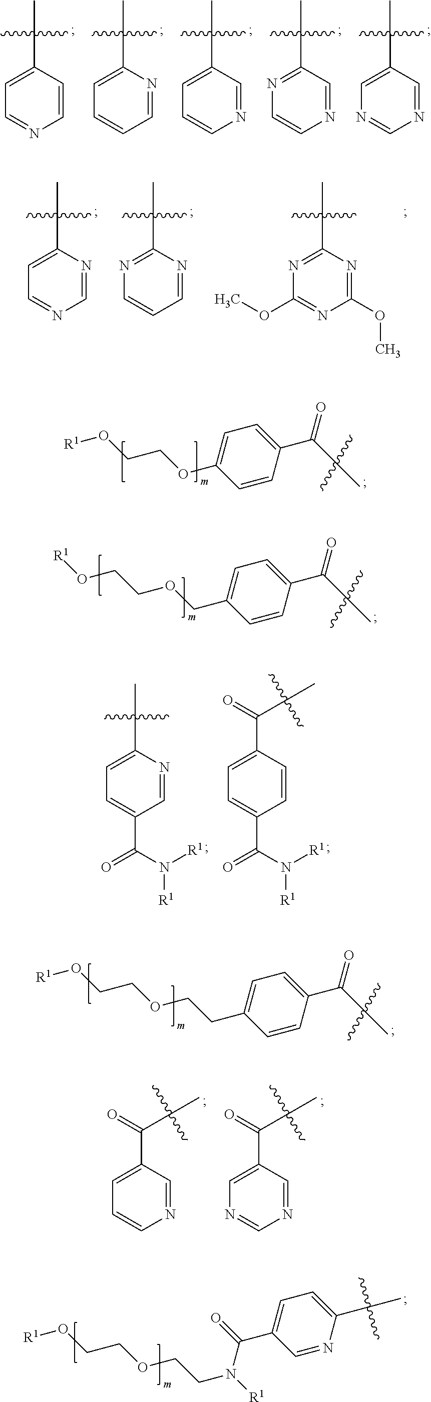

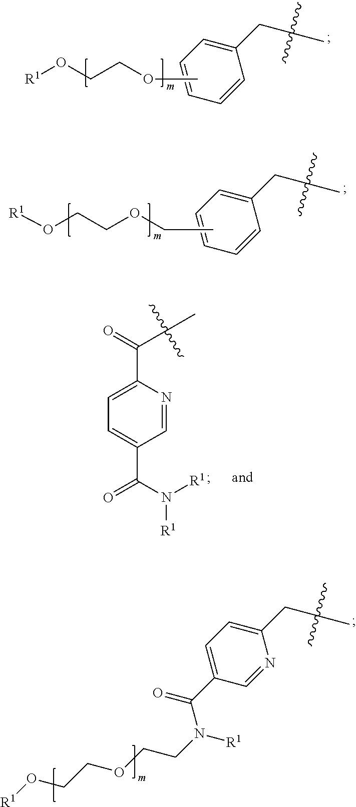

wherein subscript m is an integer from 1 to 10; R¹ is H or CH₃; R² is CH or N; and

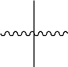

is the bond from A to Formula (III).

In some embodiments, including any of the foregoing, the polymer comprises the following structure:

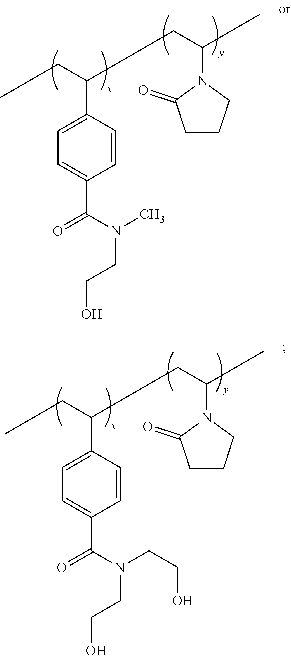

wherein the ratio of subscript x to subscript y is [0.05 to 0.25]:[0.75 to 0.95].

In some embodiments, including any of the foregoing, the polymer comprises the following structure:

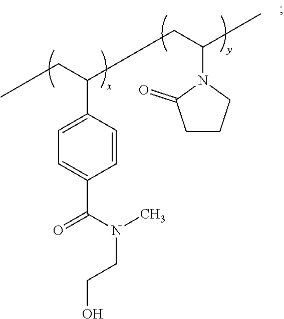

wherein the ratio of subscript x to subscript y is [0.05 to 0.25]:[0.75 to 0.95].

In some embodiments, including any of the foregoing, the polymer comprises the following structure:

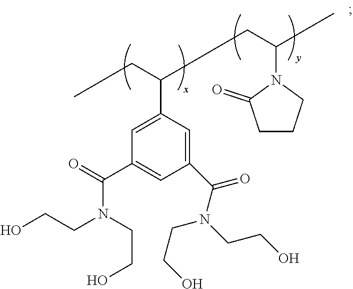

wherein the ratio of subscript x to subscript y is [0.05 to 0.25]:[0.75 to 0.95].

In some embodiments, including any of the foregoing, the polymer comprises the following structure:

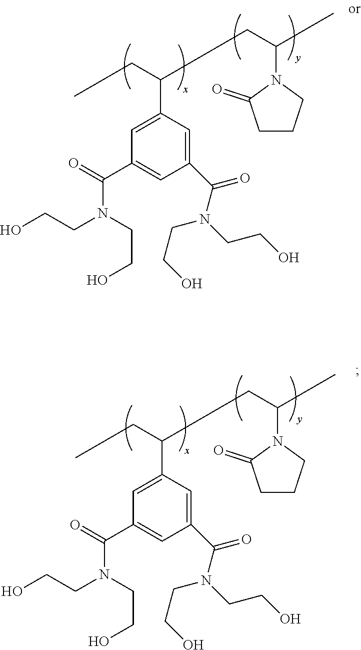

wherein the ratio of subscript x to subscript y is [0.05 to 0.25]:[0.75 to 0.95].

In some embodiments, including any of the foregoing, the ratio of subscript x to subscript y is [0.15 to 0.2]:[0.8 to 0.85].

In some embodiments, including any of the foregoing, the polymer comprises the following structure:

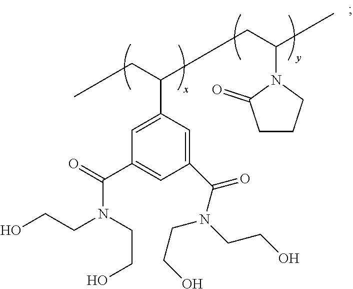

wherein the ratio of subscript x to subscript y is [0.05 to 0.25]:[0.75 to 0.95].

In some embodiments, including any of the foregoing, the polymer comprises the following structure:

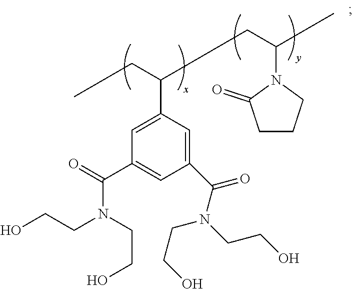

wherein the ratio of subscript x to subscript y is [0.05 to 0.25]:[0.75 to 0.95].

In some embodiments, including any of the foregoing, the polymer comprises the following structure:

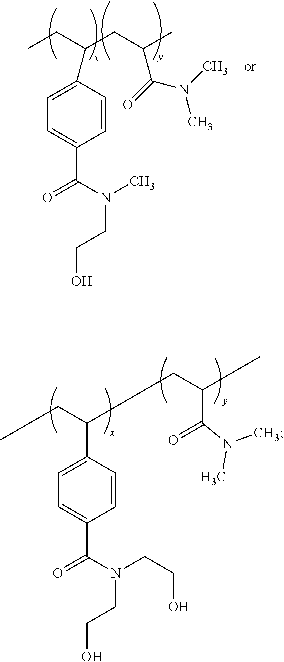

wherein the ratio of subscript x to subscript y is [0.5 to 0.75]:[0.25 to 0.5].

In some embodiments, including any of the foregoing, the polymer comprises the following structure:

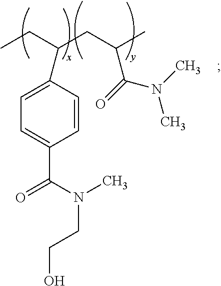

wherein the ratio of subscript x to subscript y is [0.5 to 0.75]:[0.25 to 0.5].

In some embodiments, including any of the foregoing, the polymer comprises the following structure:

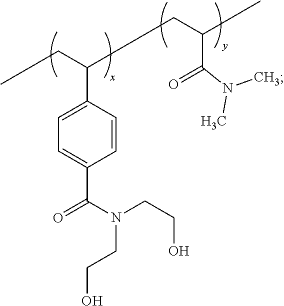

wherein the ratio of subscript x to subscript y is [0.5 to 0.75]:[0.25 to 0.5].

In some embodiments, including any of the foregoing, the polymer comprises the following structure:

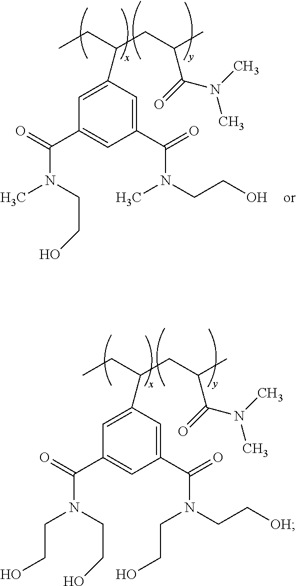

wherein the ratio of subscript x to subscript y is [0.5 to 0.75]:[0.25 to 0.5].

In some embodiments, including any of the foregoing, the polymer comprises the following structure:

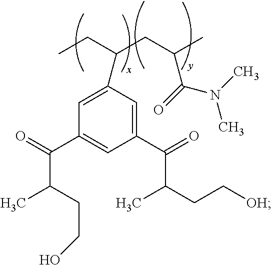

wherein the ratio of subscript x to subscript y is [0.5 to 0.75]:[0.25 to 0.5].

In some embodiments, including any of the foregoing, the polymer comprises the following structure:

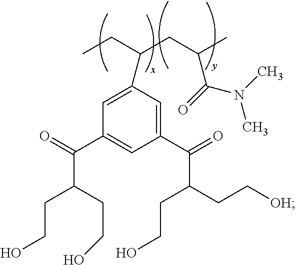

wherein the ratio of subscript x to subscript y is [0.5 to 0.75]:[0.25 to 0.5].

In some embodiments, including any of the foregoing, the ratio of subscript x to subscript y is [0.65 to 0.7]:[0.3 to 0.35].

In some embodiments, including any of the foregoing, the average molecular weight of the polymer is less than 10,000 Da.

In some embodiments, including any of the foregoing, the average molecular weight of the polymer is between 1,000-5,000 Da.

In some embodiments, including any of the foregoing, the polymer has a dispersity index less than 3.

In some embodiments, including any of the foregoing, the polymer has a dispersity index less than 2.

In some embodiments, including any of the foregoing, the polymer comprises a structure selected from Formula (IV)

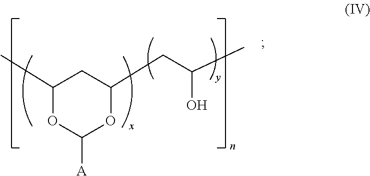

wherein A is, independently in each instance, selected from the group consisting of aryl, alkyl-aryl, heteroalkyl-aryl; heteroaryl, alkyl-heteroaryl, and heteroalkyl-heteroaryl; subscript x is an integer from 1 to 50; subscript y is an integer from 1 to 50; and subscript n is an integer from 1 to 100,000.

In some embodiments, including any of the foregoing, A is selected from:

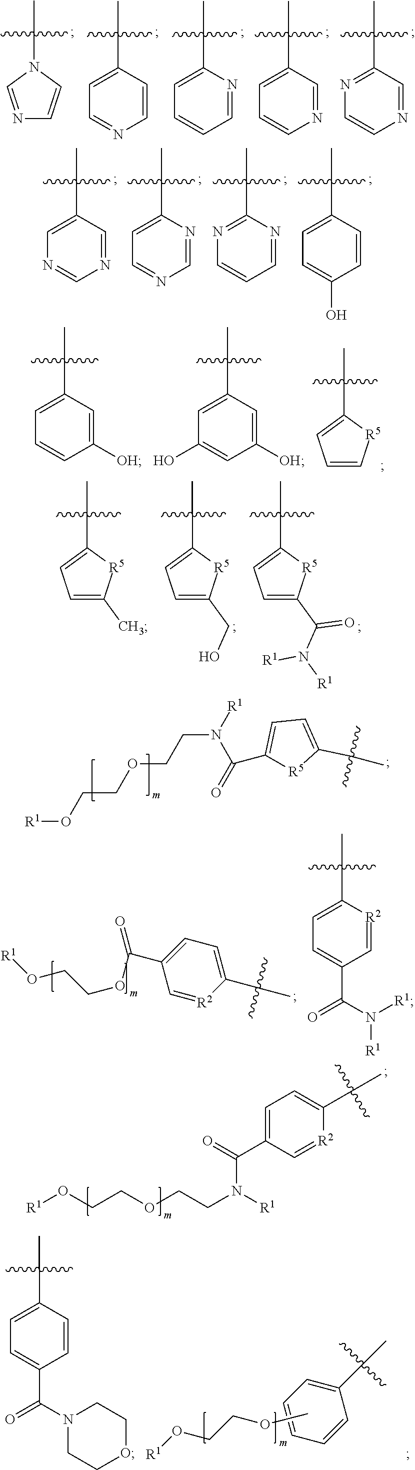

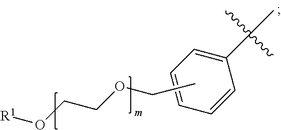

wherein subscript m is an integer from 1 to 10; R¹ is H or CH₃; R² is CH or N; R⁵ is O or S; and

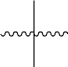

is the bond from A to Formula (III).

In some embodiments, including any of the foregoing, the polymer comprises a structure selected from Formula (V), (VI), (VII), or (VIII):

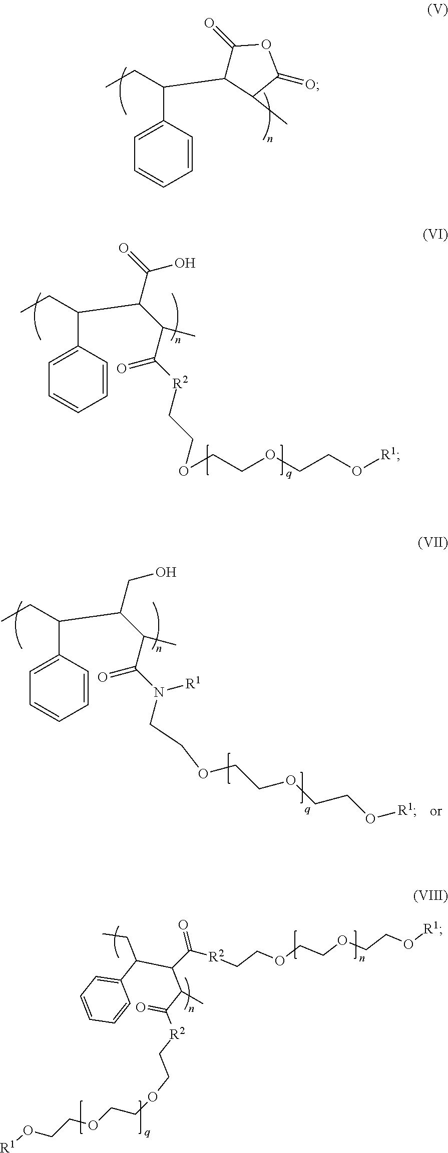

wherein subscript n is an integer from 1 to 100,000; subscript q is an integer from 1 to 50; R¹ is H or CH₃; and R² is selected from O, N, and S.

In some embodiments, including any of the foregoing, the polymer has a structure selected from

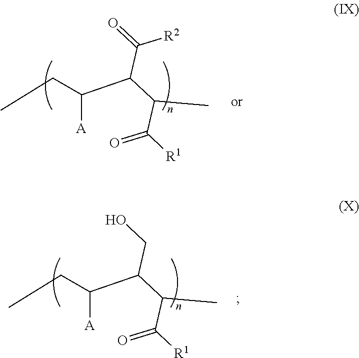

wherein A is, independently in each instance, selected from the group consisting of aryl; alkyl-aryl; aryl-alkyl, heteroalkyl-aryl; aryl-heteroalkyl; heteroaryl; alkyl-heteroaryl; heteroaryl-alkyl; heteroalkyl-heteroaryl; and heteroaryl-heteroalkyl, and subscript n is, independently in each instance, an integer from 1 to 10,000; R¹ and R² are each, individually in each instance, selected from:

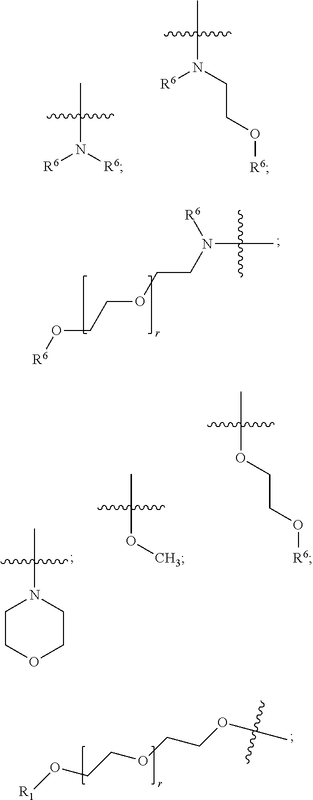

and combinations thereof; subscript r is an integer from 1 to 50; R⁶ is either H or CH₃; and

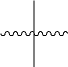

is point of attachment to structure.

In some embodiments, including any of the foregoing, A is selected from:

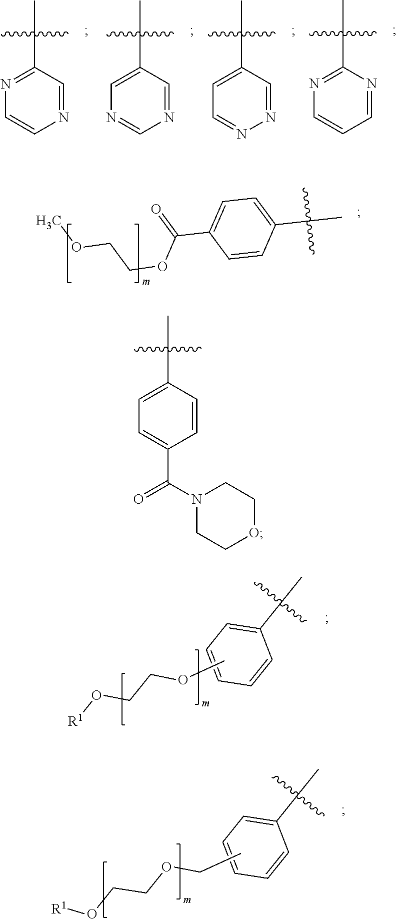

wherein subscript m is an integer from 1 to 10; R¹ is H or CH₃.

In some embodiments, including any of the foregoing, the polymer comprises a structure selected from

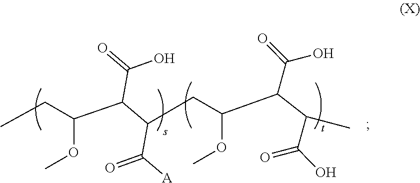

wherein A and B are each, independently in each instance, selected from the group consisting of aryl, alkyl-aryl; heteroalkyl-aryl; heteroaryl, alkyl-heteroaryl, and heteroalkyl-heteroaryl; and subscript s and t are each, independently in each instance, an integer from 1 to 10,000.

In some embodiments, including any of the foregoing, A is selected from:

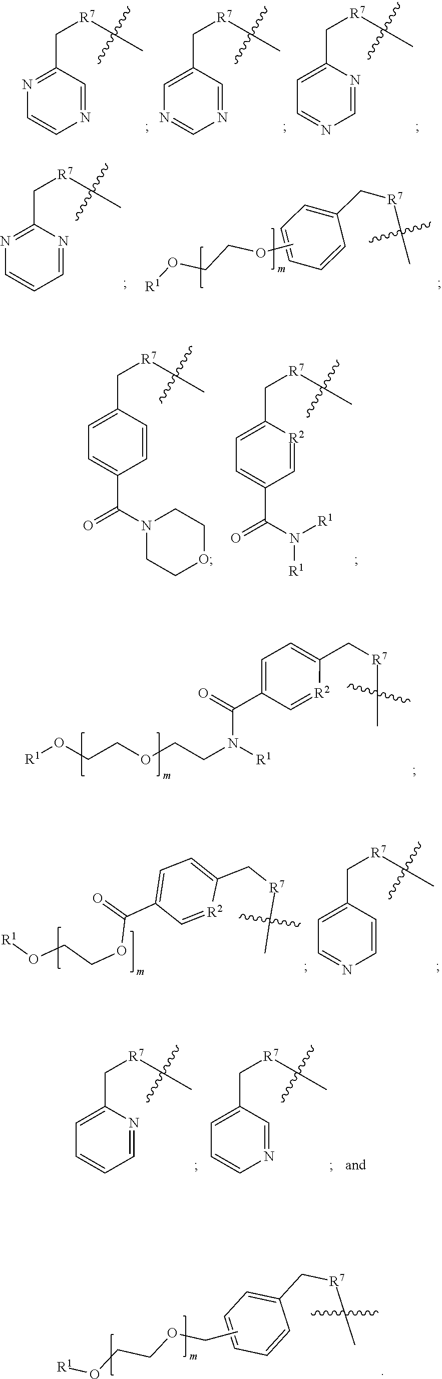

wherein subscript m is an integer from 1 to 10; R¹ is H or CH₃; R² is CH or N; and R⁷ is O, NH, or NCH₃.

In some embodiments, including any of the foregoing, the polymer comprises a structure selected from Formula XI, XII, and XIII:

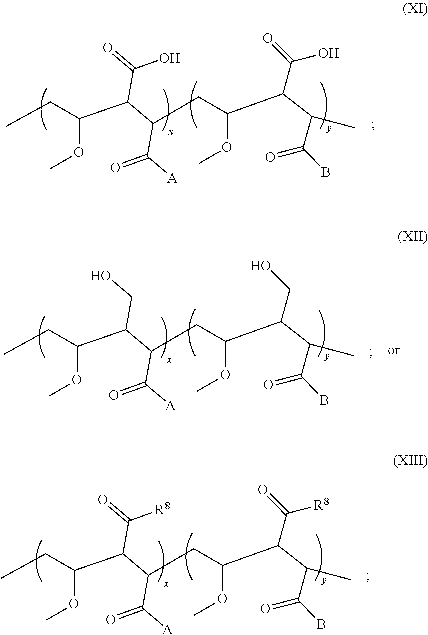

wherein subscripts u and v are each, independently in each instance, an integer from 1 to 100,000.

In some embodiments, including any of the foregoing, A and B are each, independently in each instance, selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; heteroaryl-heteroalkyl;
wherein R⁸ is selected from:

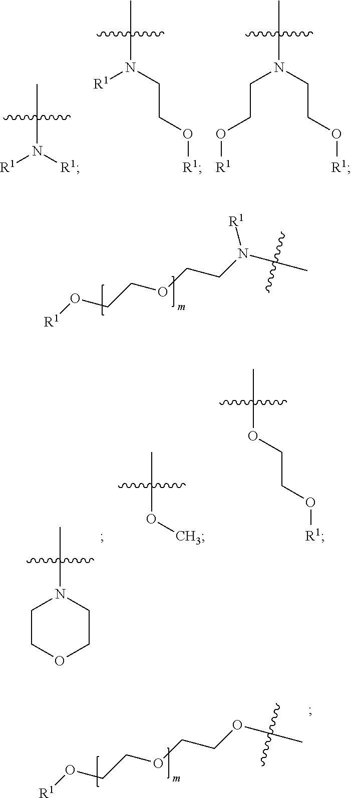

wherein subscript m is an integer from 1 to 10;
R¹ is either H or CH₃; and

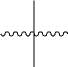

is point of attachment to structure.

In some embodiments, including any of the foregoing, the polymer comprises the following structure:

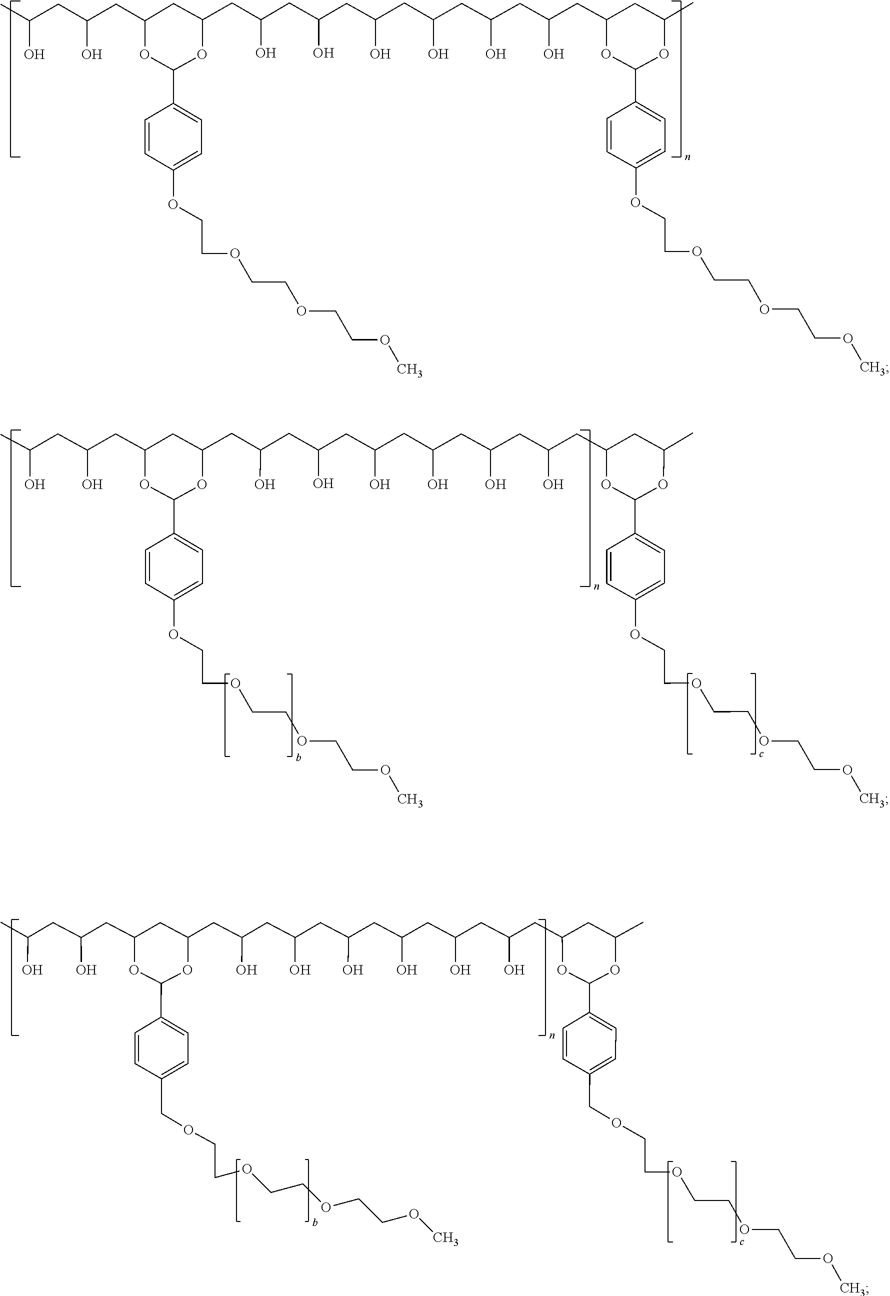

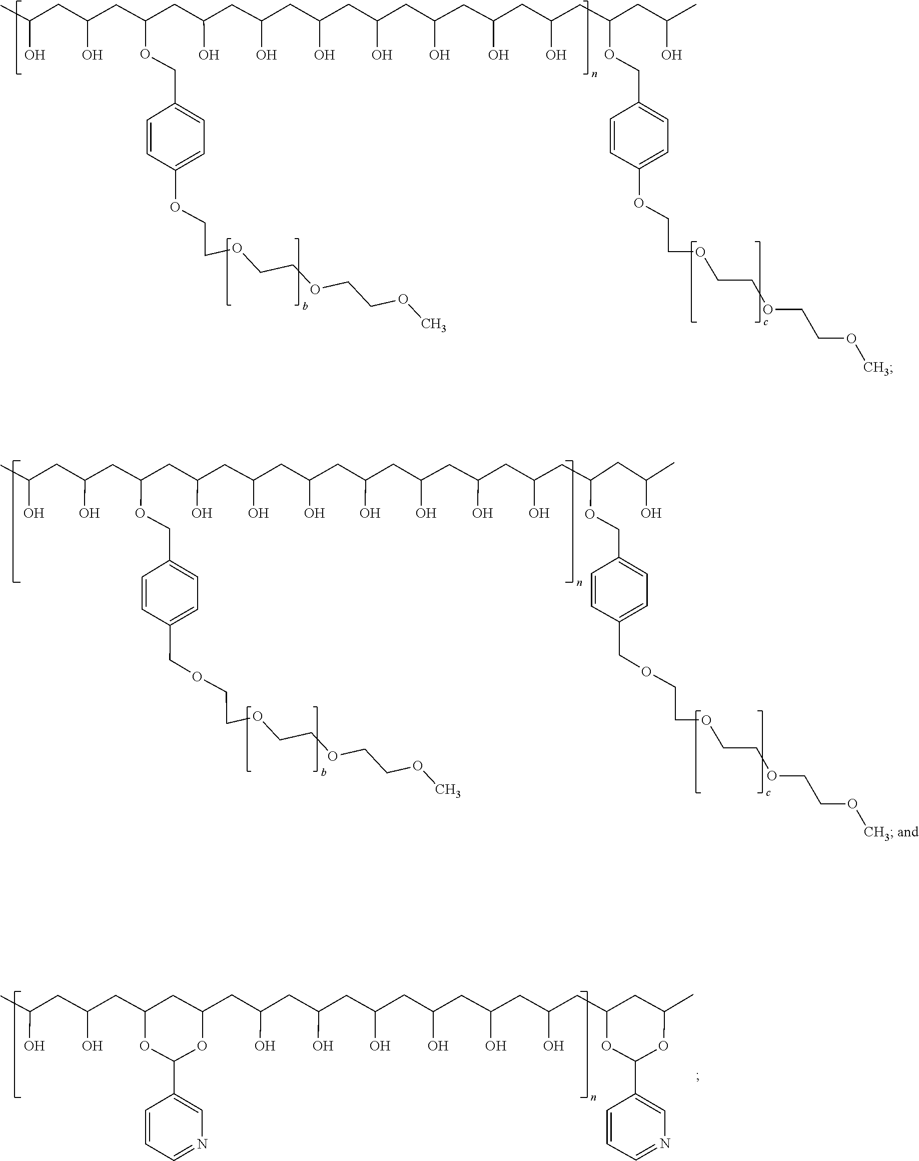

wherein subscript n is an integer from 1 to 100,000; subscript b is an integer from 0 to 6; and subscript c is an integer from 0 to 6.

In some embodiments, including any of the foregoing, the polymer does not exhibit an aqueous critical micelle concentration (CMC_{aq}).

In some embodiments, set forth is a reaction mixture, comprising: a dispersing agent and at least one fluorophore, wherein the dispersing agent comprises a polymer set forth herein, or combinations thereof.

In some embodiments, including any of the foregoing, the fluorophore comprises a polymeric dye.

In some embodiments, including any of the foregoing, the fluorophore comprises a polymeric tandem dye.

In some embodiments, including any of the foregoing, the fluorophore comprises a tandem dye.

In some embodiments, including any of the foregoing, the fluorophore is conjugated to a specific binding member that specifically binds to a target analyte.

In some embodiments, including any of the foregoing, the specific binding member is a biomolecule.

In certain embodiments, the reaction mixture is in a liquid form.

In certain other embodiments, the reaction mixture is in a dried form.

In some embodiments, including any of the foregoing, the dried form comprises a lyophilized form.

In some embodiments, including any of the foregoing, the dried form comprises a vitrified form.

In some embodiments, including any of the foregoing, the polymer is present at a percent weight-volume (w/v) of the reaction mixture of between 0.001% and 15% w/v.

In some embodiments, including any of the foregoing, the polymer is present at a percent weight-volume of the reaction mixture of between 0.05% and 2% w/v.

In some embodiments, including any of the foregoing, the polymer is present at a percent weight-volume of the reaction mixture of 0.1% and 1% w/v.

In some embodiments, including any of the foregoing, the polymer is present in the reaction mixture at a concentration of between 0.001 g/mL and 100 mg/mL.

In some embodiments, including any of the foregoing, the polymer is present in the reaction mixture at a molar ratio of between 50:1 and 1500:1 relative to the specific binding member.

In some embodiments, including any of the foregoing, the polymer is present in the reaction mixture at a molar ratio of between 800:1 and 1100:1 relative to the specific binding member, respectively. A method of labeling one or more of a target analyte present in a sample, comprising: contacting the sample with the reaction mixture set forth herein under conditions sufficient for specific binding of the target analyte to a labeled specific binding member included in the reaction mixture, thereby providing a labeling composition contacted sample.

In some embodiments, including any of the foregoing, the sample is a biological sample.

In some embodiments, including any of the foregoing, the method further includes evaluating the sample for a presence and/or amount of one or more of a target analyte, comprising: assaying a labeling composition contacted sample for the presence and/or amount of a labeled specific binding member-target analyte binding complex.

In some embodiments, set forth is a method of preventing aggregation between two or more labeled specific binding members in a solution, comprising: providing a solution of a dispersing agent comprising a polymer set forth herein, or combinations thereof, and separately adding each of the two or more labeled specific binding members to the solution to provide a labeled specific binding member-dispersing agent composition, thereby preventing aggregation between the two or more labeled specific binding members.

In some embodiments, including any of the foregoing, the method further includes drying down the labeled specific binding member-dispersing agent composition; and at a later time, reconstituting the labeled specific binding member-dispersing agent composition to provide a reconstituted labeled specific binding member-dispersing agent composition.

In some embodiments, including any of the foregoing, drying down the labeled specific binding member-dispersing agent composition includes lyophilizing or vitrifying the labeled specific binding member-dispersing agent composition.

In some embodiments, including any of the foregoing, preventing aggregation comprises a reduction in aggregation of 80% or more as compared to a case where each of the two or more labeled specific binding members are combined in absence of the dispersing agent.

In some embodiments, including any of the foregoing, the reduction in aggregation is 95% or more.

In some embodiments, including any of the foregoing, a molar ratio of the polymer to the two or more labeled specific binding members is between 50:1 and 1500:1, respectively, for preventing aggregation.

In some embodiments, including any of the foregoing, a percent w/v of polymer in the labeled specific binding member-dispersing agent composition, or the reconstituted labeled specific binding member-dispersing agent composition, is between 0.05% and 15% w/v.

In some embodiments, set forth is a method of reversing aggregation between two or more labeled specific binding members, comprising: forming a mixture of the two or more labeled specific binding members, wherein forming the mixture results in some level of aggregation between the two or more labeled specific binding members; and contacting the mixture with a dispersing agent comprising a polymer set forth herein, or combinations thereof, to provide a labeled specific binding member-dispersing agent composition, thereby reversing aggregation between the two or more labeled specific binding members.

In some embodiments, including any of the foregoing, the mixture of the two or more labeled specific binding members is in liquid form.

In some embodiments, including any of the foregoing, reversing aggregation comprises reversing 80% or more aggregation that occurred between the two or more labeled specific binding members in the mixture prior to the mixture being contacted with the composition.

In some embodiments, including any of the foregoing, reversing aggregation comprises reversing 95% or more aggregation.

In some embodiments, including any of the foregoing, a molar ratio of polymer relative to the two or more labeled specific binding members is between 50:1 and 1500:1, respectively, in the labeled specific binding member-dispersing agent composition.

In some embodiments, including any of the foregoing, a percent w/v of the polymer in the labeled specific binding member composition is between 0.05% and 15% w/v.

In some embodiments, including any of the foregoing, each of the two or more labeled specific binding members are comprised of a specific binding member conjugated to a fluorophore.

In some embodiments, set forth is a method of reducing non-specific binding between one or more labeled specific binding members and a cell, the method comprising: contacting the cell with a dispersing agent comprising a polymer set forth herein, or combinations thereof, prior to and/or during labeling of at least one target analyte associated with the cell with the one or more labeled specific binding members, thereby reducing non-specific binding.

In some embodiments, set forth is a method of reducing aggregation of a microsphere population, the method comprising: contacting the microsphere population with a dispersing agent comprising a polymer set forth herein, or combinations thereof, thereby reducing aggregation of the microsphere population as compared to an amount of aggregation of the microsphere population in absence of the contacting.

In some embodiments, including any of the foregoing, the microsphere population comprises microspheres comprised of a hydrophobic polymer.

In some embodiments, including any of the foregoing, the reduction in aggregation occurs in response to contacting the microsphere population with the composition prior to aggregation of the microsphere population or following aggregation of the microsphere population.

In some embodiments, including any of the foregoing, microspheres corresponding to the microsphere population have diameters ranging from about 5 nm to about 100 μm.

In some embodiments, including any of the foregoing, microspheres corresponding to the microsphere population are internally and/or externally labeled.

In some embodiments, set forth is a reaction mixture, comprising: a population of microspheres; and a polymer set forth herein, or combinations thereof.

In some embodiments, set forth is a reaction mixture, comprising: a wetting agent and a biological sample, wherein the wetting agent comprises a polymer set forth herein, or combinations thereof.

In some embodiments, including any of the foregoing, the biological sample comprises at least one cell.

In some embodiments, set forth is a kit comprising: a reaction mixture set forth herein and, optionally, a reconstitution buffer.

In some embodiments, set forth is a kit that includes: a dispersing agent comprising a polymer set forth herein, or combinations thereof, and at least one labeled specific binding member in a liquid or a dried form.

In some embodiments, including any of the foregoing, the kit includes a population of microspheres in solution.

### EXAMPLES

The following examples are put forth so as to provide those of ordinary skill in the art with a complete disclosure and description of how to make and use the methods and compositions of the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Illustrative Image of a Dispersant Solution of the Present Disclosure

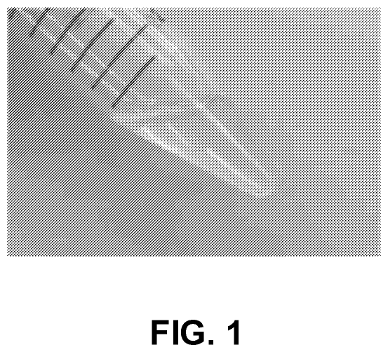

This Example demonstrates that the dispersants of the present disclosure are clear and colorless at w/v percentages used in the methods described herein. Specifically, a 10% w/v solution of dispersant PDD-301 was prepared. FIG. 1 is an image of the prepared solution, demonstrating the clear and colorless nature of the solution.

### Example 2. Dye Aggregation Preventability and Reversibility

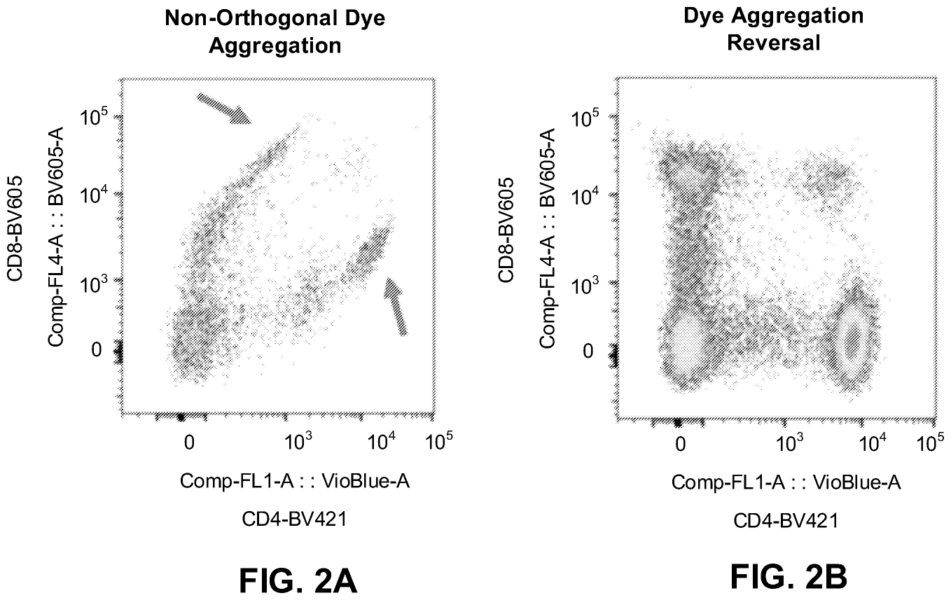

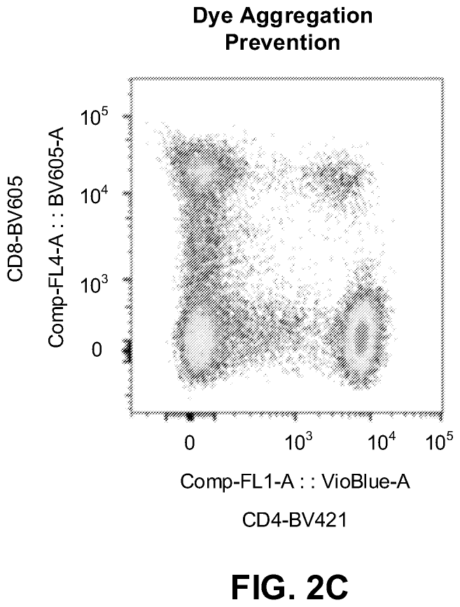

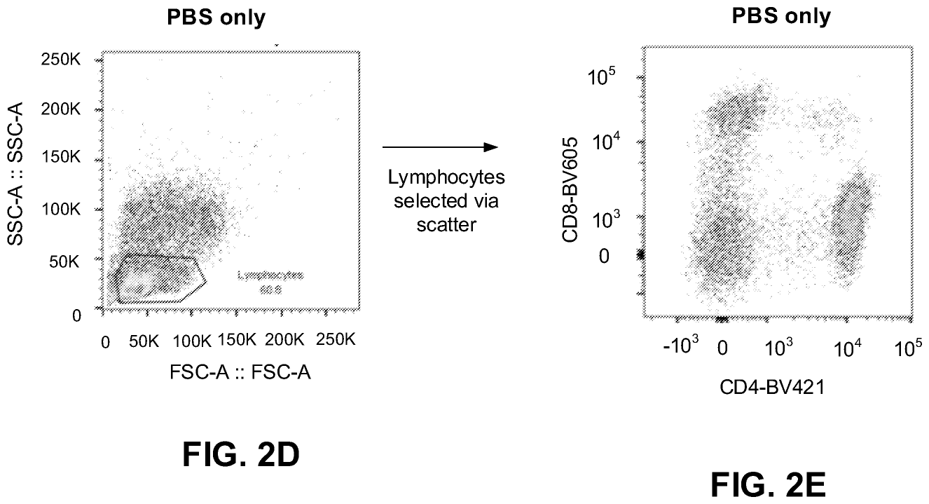

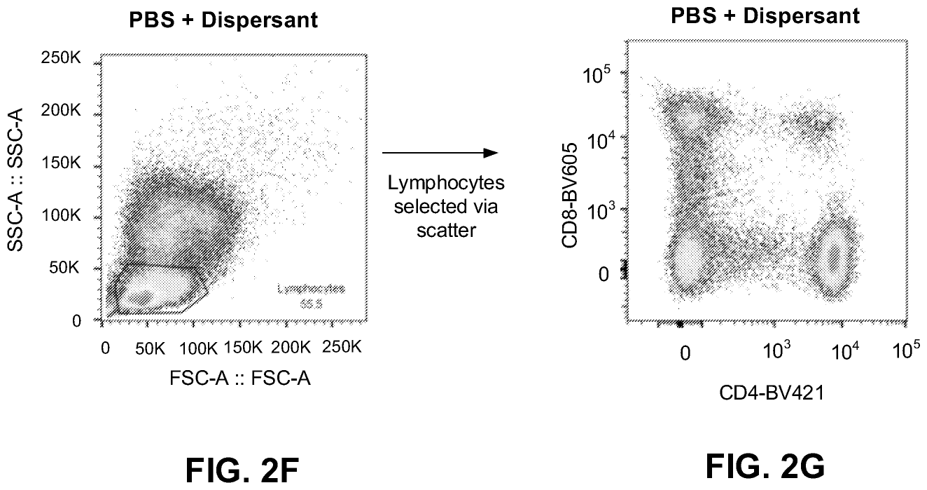

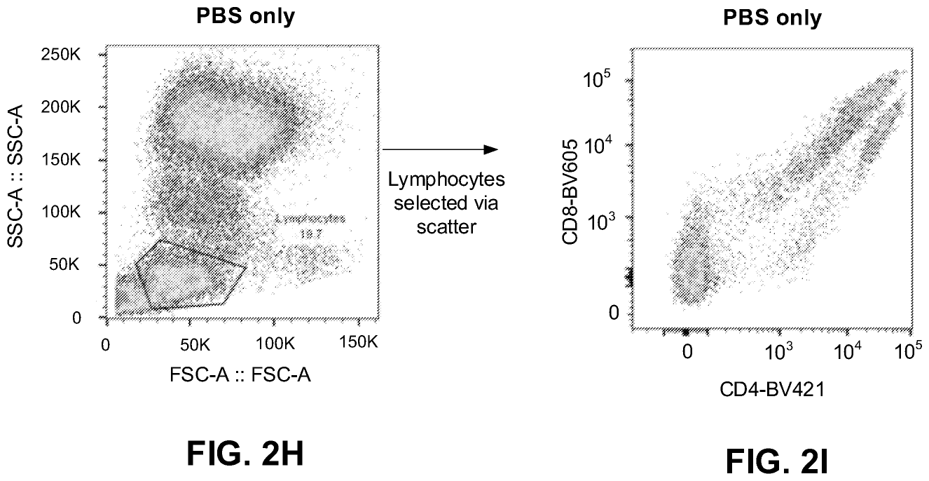

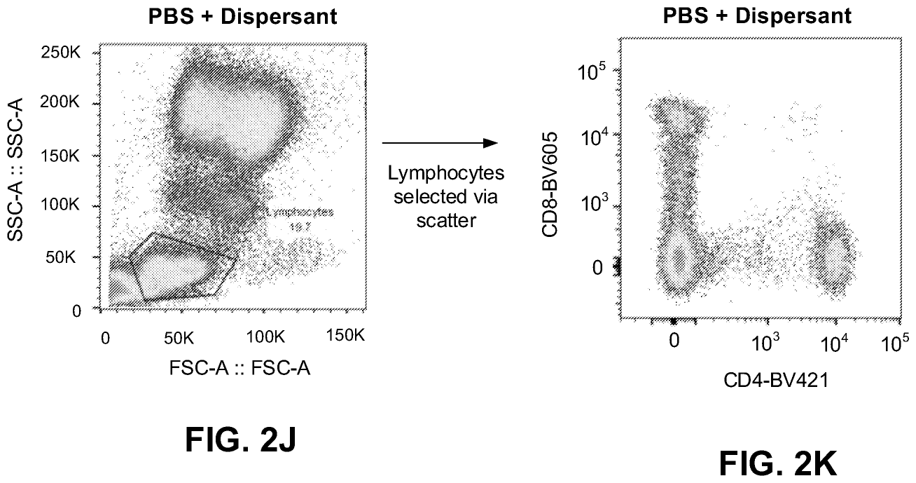

This Example demonstrates that the dispersants of the present disclosure are capable of preventing aggregation of labeled specific binding members, as well as being capable of reversing aggregation of labeled specific binding members. In this Example, the labeled specific binding members comprise the polymer dye conjugates CD4-BV421 (Cat #300531, BioLegend, Inc., San Diego, CA) and CD8-BV605 (Cat #344741, BioLegend, Inc., San Diego, CA). In a first experiment, a cocktail of the two polymer dye conjugates was prepared and diluted into PBS lacking any dispersants of the present disclosure. The PBS solution containing the cocktail of polymer dye conjugates was allowed to sit at room temperature for one hour, before the addition of peripheral blood mononuclear cells (PBMCs) (Cat #PF3201, First Choice Bio, LLC, Benicia, California), to label CD4+ and CD8+ cells with the CD4-BV421 and CD8-BV605 polymer dye conjugates, respectively. Following the labeling procedure, the cells were subjected to flow cytometry analysis. As illustrated at FIG. 2A, when the labeling was conducted with polymer dye conjugates that had not been contacted with any dispersant of the present disclosure, clear aggregation of the polymer dye conjugates is observed.

The polymer dye conjugate aggregation seen in the experimental results shown at FIG. 2A is reversible. Specifically, in a second experiment, again a cocktail of the two polymer dye conjugates CD4-BV421 and CD8-BV605 was prepared and diluted into PBS lacking any dispersants of the present disclosure. The prepared solution containing the two polymer dye conjugates was allowed to sit at room temperature for one hour. Following the room temperature incubation period, a volume of 100 mg/mL PDD-301 in PBS was added to a final concentration of 5 mg/mL, mixed, and allowed to sit for 10 minutes. Subsequent to the addition of the dispersant, a sample of PBMCs were added to label CD4+ and CD8+ cells with the CD4-BV421 and CD8-BV605 polymer dye conjugates, respectively. Following the labeling procedure, the cells were subjected to flow cytometry analysis. As illustrated at FIG. 2B, addition of the PDD-301 dispersant reversed polymer dye conjugate aggregation, leading to spectrally pure signatures corresponding to labeled CD4 and CD8 cell populations with the corresponding polymer dye conjugates. For reversal of aggregation, although not specifically illustrated time frames that the dispersant and polymer dye conjugates may be allowed to sit to achieve aggregation reversal comprise about 5-60 minutes, for example about 5-30 minutes, for example about 10-20 minutes.

The polymer dye conjugate aggregation seen in the experimental results of FIG. 2A is also preventable. Specifically, in a third experiment a volume of 100 mg/mL PDD-301 dispersant in PBS was added to a final concentration of 5 mg/mL in PBS. Next, the polymer dye conjugates CD4-BV421 and CD8-BV605 were added to the solution of PBS plus dispersant. The prepared sample was allowed to sit for one hour at room temperature, prior to the addition of PBMCs to label CD4+ and CD8+ cells with the CD4-BV421 and CD8-BV605 polymer dye conjugates, respectively. Following the labeling procedure, the cells were subjected to flow cytometry analysis. As illustrated at FIG. 2C, polymer dye conjugate aggregation was prevented in the presence of the dispersant, leading to spectrally pure signatures corresponding to labeled CD4 and CD8 cell populations with the corresponding polymer dye conjugates.

Additional evaluation data is provided herein for illustrative purposes. For the experiments, both PBMC samples and whole blood samples were used. For PBMC samples, freshly thawed PBMCs were washed and resuspended in PBS immediately prior to use. Staining of cells with antibodies (CD-BV421 and CD8-BV605 mAbs) was conducted by incubation at room temperature for twenty minutes. PBMCs were washed twice with PBS, and then resuspended in 1% PFA PBS for flow analysis. For whole blood samples, fresh whole blood anticoagulated with EDTA was utilized. Staining of cells with antibodies (CD-BV421 and CD8-BV605 mAbs) was completed by incubation at RT for 20 minutes. Post staining lysis of red blood cells (RBCs) was completed by 1:20 dilution with FACS Lyse (10 minutes). Cells were washed with PBS and then resuspended in 1% PFA PBS for flow analysis.

FIGS. 2D-2E illustrate flow cytometry data obtained from PBMC samples in absence of dispersant, and FIGS. 2F-2G illustrate flow cytometry data obtained from PBMC samples in presence of 5 mg/mL PDD-301 dispersant. FIGS. 2H-2I illustrate flow cytometry data obtained from whole blood samples in absence of dispersant, and FIGS. 2J-2K illustrate flow cytometry data obtained from whole blood samples in presence of 5 mg/mL PDD-301 dispersant. As illustrated, the use of dispersants resulted in substantially less aggregation as measured by flow cytometry as compared to samples similarly prepared and analyzed in absence of dispersant.

It may thus be understood that the dispersants of the present disclosure can be included in wash/flow buffer components for flow cytometry analysis, for example samples can be washed with PBS containing e.g., 0.1% w/v dispersant(s), and additionally or alternatively samples can be suspended in PBS containing, e.g., 1% PFA, 0.1% w/v dispersant(s) for the flow aspect of the analysis.

### Example 3. Prevention of Dye Aggregation Over Extended Time Periods

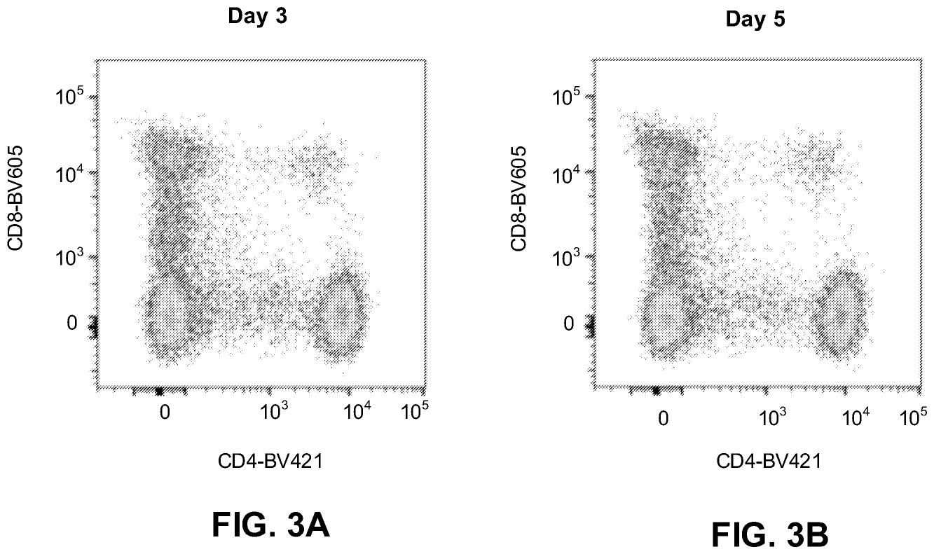

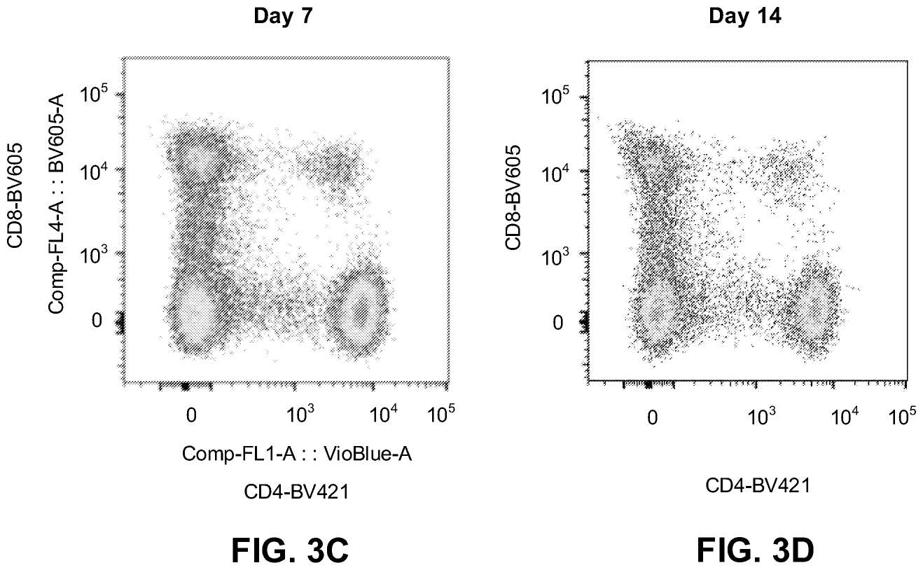

This Example demonstrates that polymer dye conjugate aggregation can be prevented over time frames of at least 14 days. In this Example, an experimental procedure similar to that discussed above at FIG. 2C was conducted. Briefly, a volume of 100 mg/mL PDD-301 in PBS was added to a final concentration of 5 mg/mL in PBS, followed by addition of the polymer dye conjugates CD4-BV421 and CD8-BV605. The solutions containing the dispersant and the polymer dye conjugates was allowed to sit at room temperature for either 3 days, 5 days, 7 days, or 14 days prior to the addition of PBMCs to label CD4+ and CD8+ cells with the CD4-BV421 and CD8-BV605 polymer dye conjugates, respectively. As shown in FIGS. 3A-3D, spectrally pure signatures corresponding to labeled CD4 and CD8 cell populations were obtained from a flow cytometry analysis following the incubation of the solutions containing polymer dye conjugates and dispersant for 3 days (FIG. 3A), 5 days (FIG. 3B), 7 days (FIG. 3C), or 14 days (FIG. 3D) at room temperature prior to addition of PBMCs.

### Example 4. Increased Cell Recovery in Presence of Dispersants

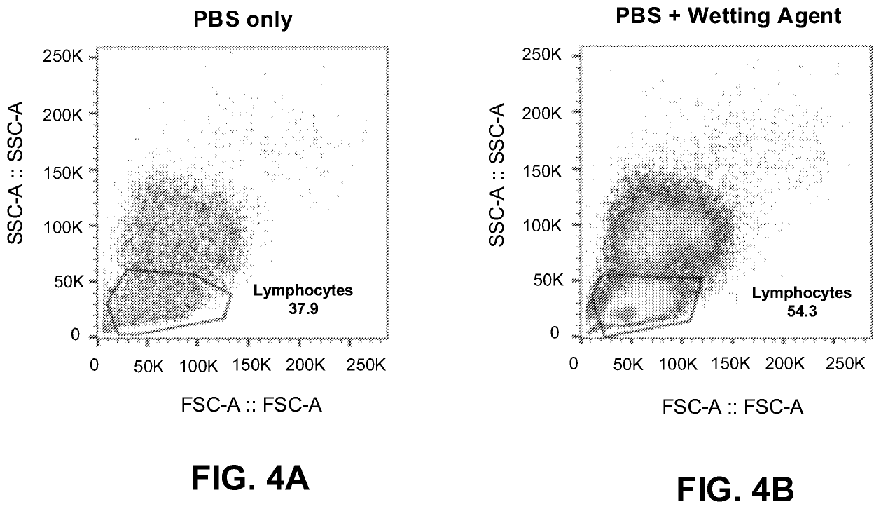

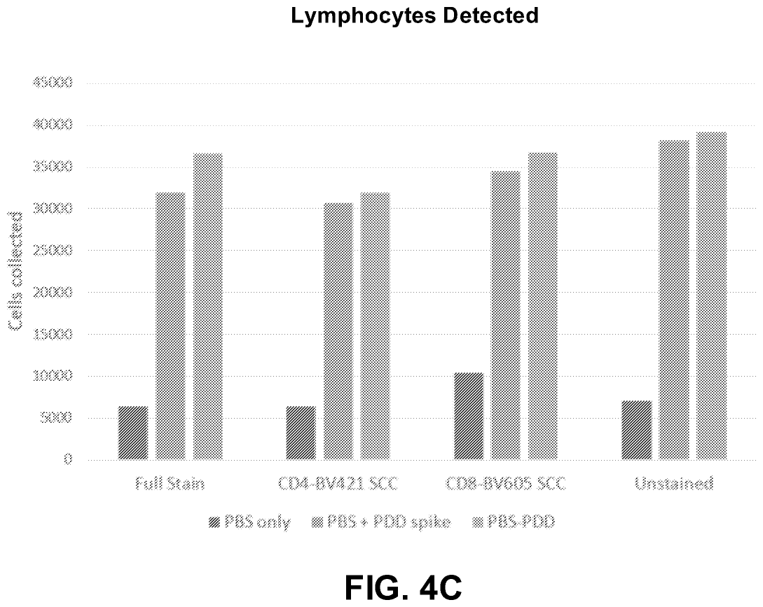

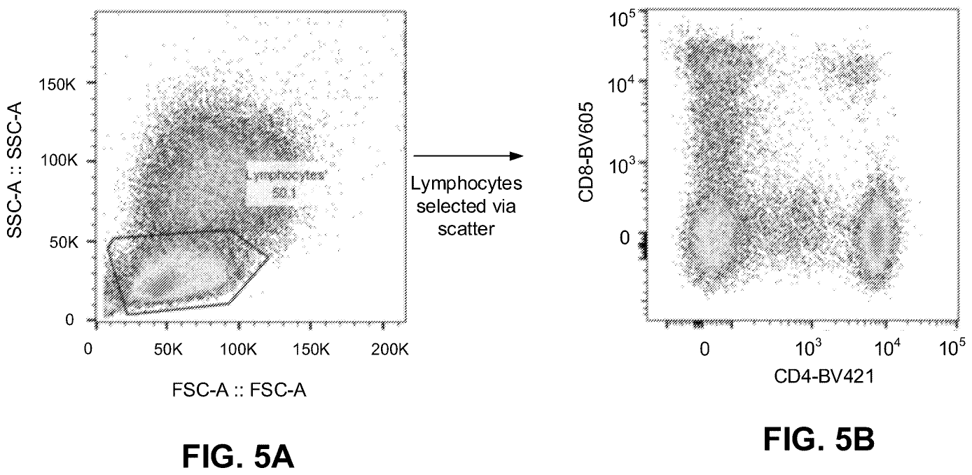

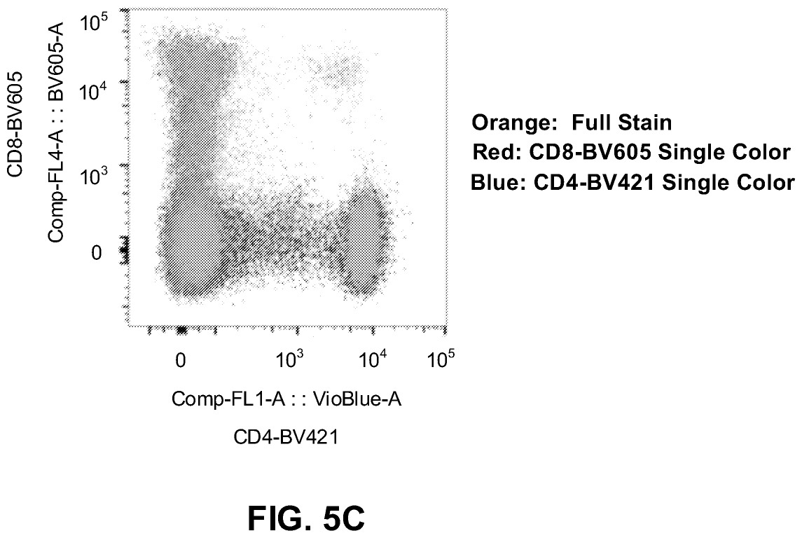

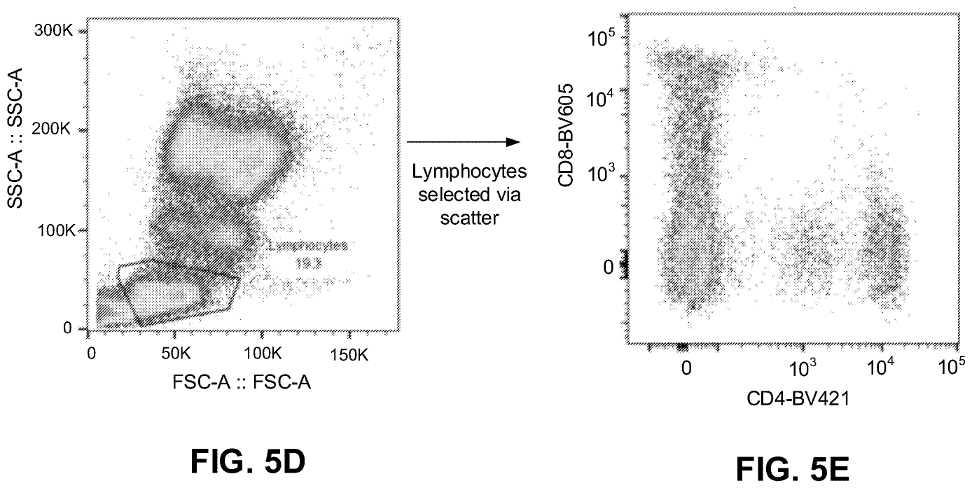

This Example demonstrates that recovery of cells used in experimental procedures such as flow cytometry analysis can be increased via the use of at least one wetting agent of the present in such experimental procedures. FIGS. 4A-4B show example scatter plots illustrating the recovery of lymphocytes as measured by flow cytometry analysis is increased when PBS buffers include 1 mg/mL of PDD-301 (FIG. 4B), as compared to PBS buffers lacking any such wetting agent (FIG. 4A). In regard to FIGS. 4A-4B, forward scatter (FCS-A::FCS-A) corresponds to the x-axis and side scatter (SSC-A::SSC-A) corresponds to the y-axis. As shown, recovery of lymphocytes is nearly doubled when buffers used include at least one wetting agent of the present disclosure.

FIG. 4C graphically illustrates that the use of a wetting agent as herein described increases the recovery of cells after multiple wash steps. Conditions tested included the use of buffers with PBS only, PBS+1 mg/mL PDD-301 spike, and PBS+1 mg/mL PDD-301. More specifically, the PBS+1 mg/mL PDD-301 spike condition refers to an experimental design where a cocktail of CD4-BV421 and CD8-BV605 polymer dye conjugates were added to PBS, and then 1 mg/mL PDD-301 was added to reverse any aggregation. Alternatively, the PBS+1 mg/mL PDD-301 condition refers to an experimental design where the 1 mg/mL PDD-301 was added to PBS prior to addition of a cocktail of CD4-BV421 and BD8-BV605. As shown at FIG. 4C, recovery was greatest for all PDD-301 conditions as measured via flow cytometry analysis (full stain, CD4-BV421 single color controls (SCCs), CD8-BV605 SCCs, and unstained cells), with comparable recovery observed for the PBS+1 mg/mL PDD-301 spike condition.

### Example 5. Prevention of Polymer Dye Aggregation in Dried Compositions

This Example demonstrates that dispersants of the present disclosure can be vitrified along with polymer dye conjugates, such that upon reconstitution in a cell-containing buffer, a lack of dye-dye interaction is observed.

This Example also demonstrates that vitrified samples of polymer dye conjugates can be formulated with a dispersant (e.g., as described herein) comprising a major mass component of the resultant film. This Example also demonstrates that vitrified samples of polymer dyes and dispersants of the present disclosure can be obtained with a high glass transition temperature (Tg). This Example also demonstrates that biological conformation of the antibodies conjugated to the polymer dyes is preserved (i.e., the antibodies are not denatured) during the process of vitrifying and reconstituting the samples. This Example also demonstrates that there is minimal spectral contribution corresponding to the dispersants from about 350 to about 850 nm, even at high w/v percentages. This Example also demonstrates that the dispersants of the present disclosure can out-compete polymer dye-dye interactions. Finally, this Example also demonstrates that the dispersants of the present disclosure can prevent non-specific binding of polymer dye conjugates to cells. Although not specifically disclosed, similar findings were observed with lyophilized samples.

### Example 6. Methods of Making Certain Compounds

Synthetic procedures for making polymers were adapted and modified in order to arrive at the instantly claimed compounds.

For example, certain methods in Lindhoud, et al., SMA-SH: Modified Styrene-Maleic Acid Copolymer for Functionalization of Lipid Nanodiscs, Biomacromolecules 2016, 17, 1516-1522, http://dx.doi.org/10.1021/acs.biomac.6b00140, were adapted to make certain claimed compounds. The entire contents of this publication are herein incorporated by reference in its entirety for all purposes. This includes processes for post polymerization modification of maleic anhydride copolymers (e.g., styrene or methylvinyl ether copolymers). In some examples, this includes a reaction such as Nucleophile+TEA in DMF.

Also, for example, Ugo, et al., Poly(2-vinylpyrazine) as a Soluble Polymeric Ligand and as an Electrode Coating, Reactions with Pentacyanoferrate(II), Anal. Chem. 1989, 61, 1799-1805, 0003-2700/89/0361-1799, were adapted to make certain claimed compounds. The entire contents of this publication are herein incorporated by reference in its entirety for all purposes. This includes processes for radical polymerization (AIBN as initiator) to obtain poly(2-vinylpyrazine) or other polyaromatics.

Also, for example, Gousse, et al., ACETALIZATION OF POLYVINYL ALCOHOL WITH FURFURAL, Eur. Polym. J. Vol. 33, No. 5, pp. 667-671, 1997, were adapted to make certain claimed compounds. The entire contents of this publication are herein incorporated by reference in its entirety for all purposes. This includes processes for obtaining acetylated PVA derivatives modified to incorporate pegylated aromatics or other water-soluble aromatics of the present disclosure.

Also, for example, Duncan, et al., Preparation and characterization of alkylated poly(vinyl alcohol) hydrogels using alkyl halides, Journal of Biomaterials Science, Polymer Edition, 7:8, 647-659, DOI: 10.1163/156856296X00426, were adapted to make certain claimed compounds. The entire contents of this publication are herein incorporated by reference in its entirety for all purposes. This includes processes for the alkylation or arylation of PVA modified to incorporate pegylated aromatics or other water-soluble aromatics of the present disclosure. In some examples, potassium or sodium tert-butoxide was used as the deprotonating agent.

Also, for example, Nichifor, et al., Copolymers of N-alkylacrylamides and styrene as new thermosensitive materials, Polymer. Vol. 44, No. 10, pp. 3053-3060, 2003, were adapted to make certain claimed compounds. The entire contents of this publication are herein incorporated by reference in its entirety for all purposes. This includes processes for radical polymerization (AIBN as initiator) to obtain copolymers of N-alkylacrylamides. The processes are modified to include water-soluble styrene derivatives or other water-soluble polyaromatics of the present disclosure.

Also, for example, Vijaykumar, et al., Copolymerization of N-Vinyl Pyrrolidone with Functionalized Vinyl Monomers: Synthesis, Characterization and Reactivity Relationships, Macromolecular Research. Vol. 17, No. 12, pp. 1003-1009, 2009, were adapted to make certain claimed compounds. The entire contents of this publication are herein incorporated by reference in its entirety for all purposes. This includes processes for radical polymerization (AIBN as initiator) to obtain copolymers of N-Vinyl Pyrrolidone. The processes are modified to include water-soluble styrene derivatives or other water-soluble polyaromatics of the present disclosure.

### Example 7. Methods of Making Certain Compounds

The following compound, entitled PDD-301, was made as follows:

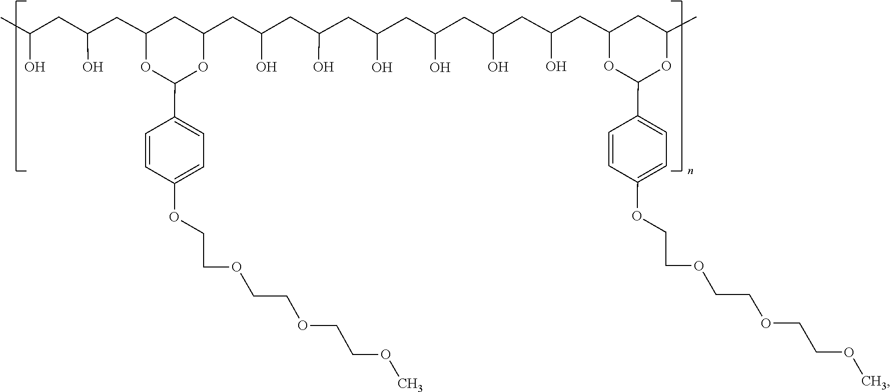

wherein n is an integer from 5 to 20.

400 mg of poly(vinyl alcohol) with an average MW of 3,000 Da (Cat #336, Scientific Polymer Products Inc., Ontario, NY) was dissolved in 4 mL of deionized water. 120 mg of m-PEG₄-benzaldehyde (Cat #BP-21100, BroadPharm, San Diego, CA), which equates to 0.1 molar equivalents of hydroxyl units present on the poly(vinyl alcohol) in solution, was added and dissolved in the mixture. The pH was titrated to 1.7 with methanesulfonic acid (Cat #471356, SigmaAldrich, St. Louis, MO) and the solution was mixed at room temperature for 24 hours. The reaction was quenched by adding triethylamine (Cat #T0886, SigmaAldrich, St. Louis, MO) to bring the pH to 10. The polymer was precipitated by adding acetone (Cat #179973, SigmaAldrich, St. Louis, MO) to the aqueous mixture and collecting via centrifugation. The precipitate was washed with acetone and then dried under vacuum for 12 hours. The material was used without any further purification.

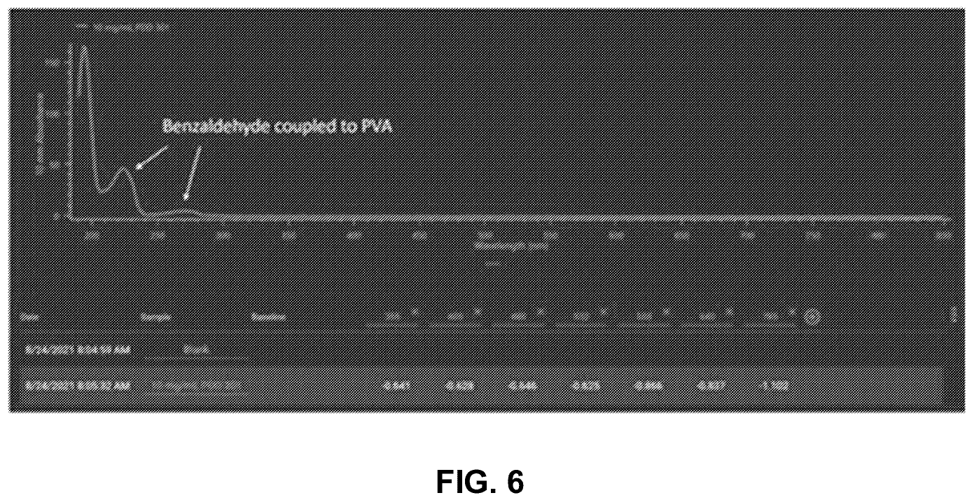

A UV-VIS spectra (FIG. 6) confirmed that benzaldehyde was successfully conjugated to PVA by the appearance of two absorbance peaks (˜225 nm and ˜275 nm) in the UV-VIS spectra of the obtained polymer that correspond to anisole. The spectra was acquired on a NanoDrop One offered by Thermo. https://www.thermofisher.com/order/catalog/product/ND-ONE-W#/ND-ONE-W. The Path length was 1.0 mm. The spectra was taken in deionized water.

### Example 8. Methods of Making Certain Compounds

The following compound was made as follows:

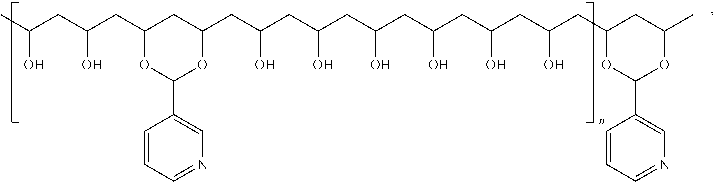

wherein n is an integer from 5 to 20.

400 mg of poly(vinyl alcohol) with an average MW of 3,000 Da (Cat #336, Scientific Polymer Products Inc., Ontario, NY) was dissolved in 4 mL of deionized water. 41.7 uL of 3-pyridinecarboxaldehyde (Cat #P62208, SigmaAldrich, St. Louis, MO), which equates to 0.1 molar equivalents of hydroxyl units present on the poly(vinyl alcohol) in solution, was added and mixed. The pH was titrated to 1.7 with methanesulfonic acid (Cat #471356, SigmaAldrich, St. Louis, MO) and the solution was mixed at room temperature for 24 hours. The reaction was quenched by adding triethylamine (Cat #T0886, SigmaAldrich, St. Louis, MO) to bring the pH to 10. The polymer was precipitated by adding acetone (Cat #179973, SigmaAldrich, St. Louis, MO) to the aqueous mixture and collecting via centrifugation. The precipitate was washed with acetone and then dried under vacuum for 12 hours. The material was used without any further purification.

### Example 9. Methods of Making Certain Compounds—Prophetic Example

The following compounds would be made as follows:

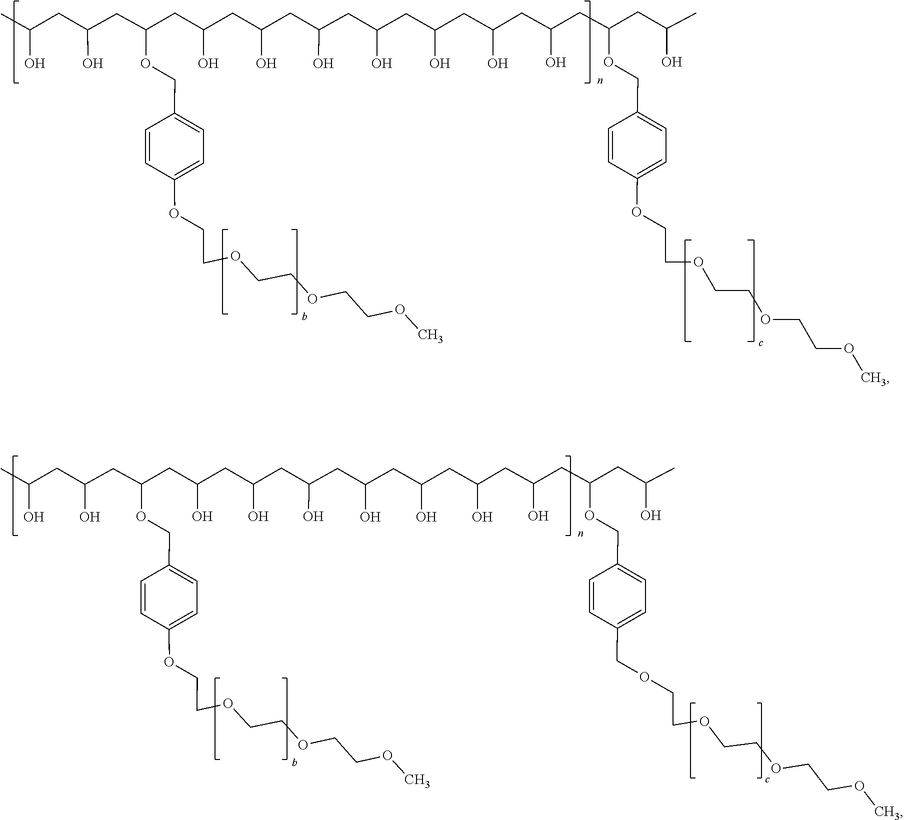

wherein n is an integer from 5 to 20.

Poly(vinyl alcohol) with a preferred average MW between 3,000 Da and 10,000 Da is dissolved in anhydrous DMSO. To the mixture is added a mPEGylated benzyl halide, mesylate, or tosylate at a 0.1-0.2× molar ratio to the amount of hydroxyl units presented by the poly(vinyl alcohol) in the solution. A 0.2× molar quantity of Potassium tert-butoxide is dissolved in anhydrous THF and is then slowly added drop wise to the mixing solution. The mixture is then heated to 80° C. for 2 hours. The reaction may also be monitored by TLC and stopped when completed. Any residual base is then quenched by the addition of acetic acid. The polymer is then obtained by precipitation and washing in acetone. The polymer can then be used directly or further purified via dialysis or chromatographic methods.

### Example 10. Methods of Making Certain Compounds

The following compound was made as follows:

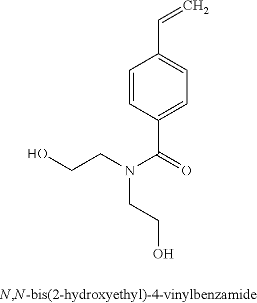 [SMILES: C=Cc1ccc(C(=O)N(CCO)CCO)cc1]

(C₁₃H₁₇NO₃), 235.28 g/mole, Ambeed catalog No. A2192458.

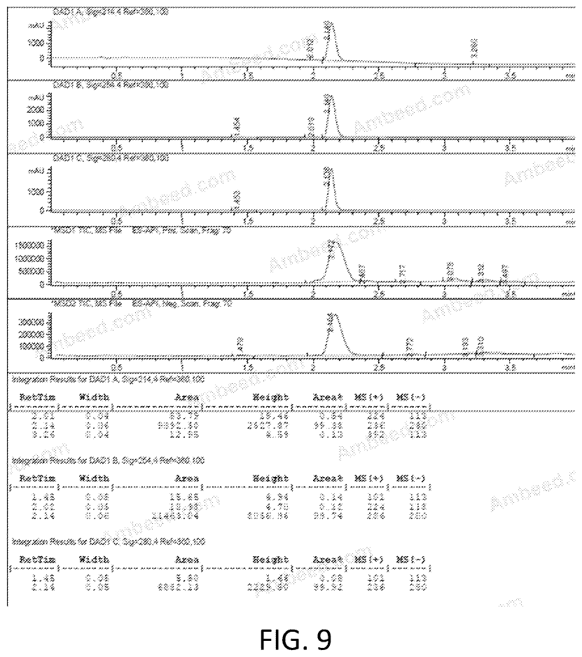

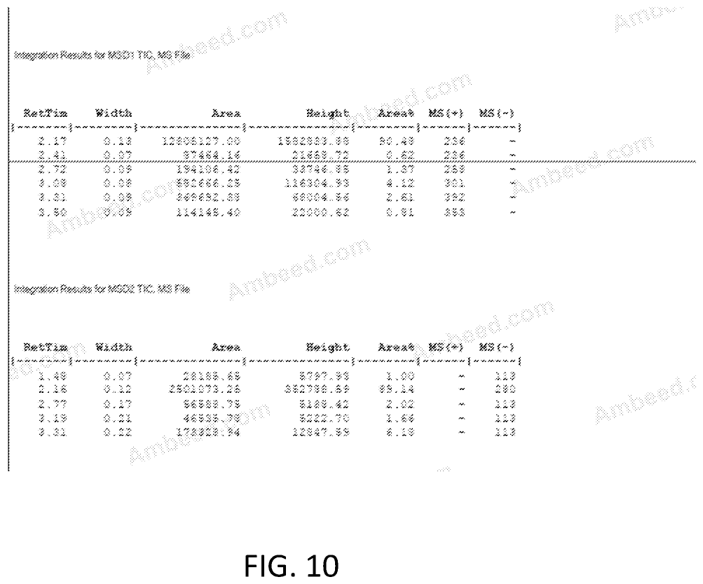

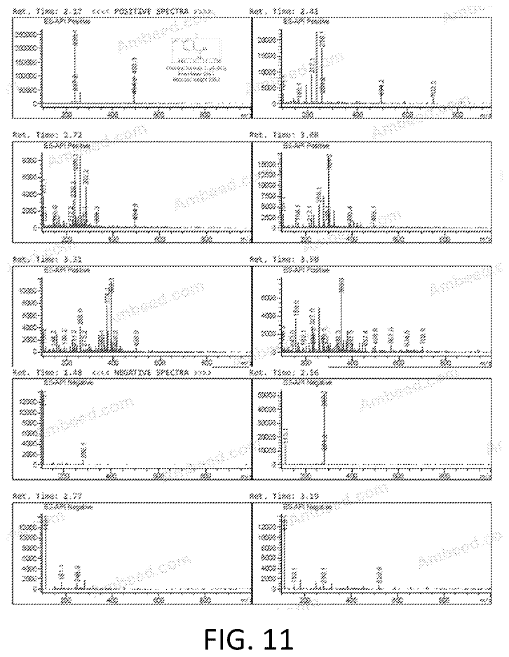

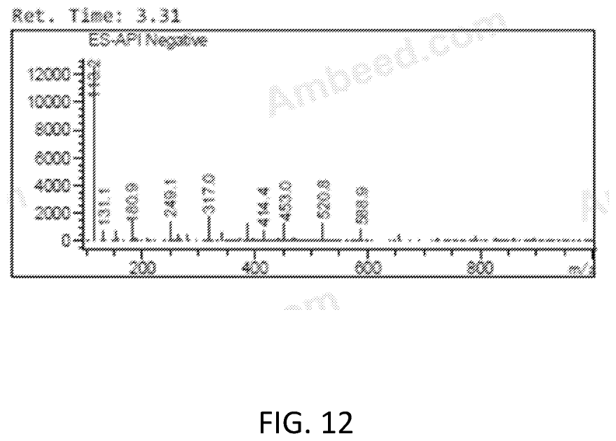

4-Vinylbenzoyl chloride (1.00 eq) was dissolved in THF (0.2 M solution) at 0° C. and allowed to stir for 10 min. Diethanolamine (2.20 eq) was then added dropwise and the reaction was warmed to room temperature and mixed overnight (18 hr) under nitrogen at which time the reaction was diluted with EtOAc, washed with 1 M NaOH and brine. The combined organic layers were dried over anhydrous Na₂SO₄, filtered, concentrated in vacuo, and the resulting residue was by flash column chromatography (EtOAc/hexanes 2:1) to afford the titled compound. ¹H NMR: NMR peaks—1H NMR (400 MHz, cd3od) δ 7.51 (d, J=8.1 Hz, 2H), 7.42 (d, J=8.0 Hz, 2H), 6.77 (dd, J=17.6, 11.0 Hz, 1H), 5.85 (d, J=17.6 Hz, 1H), 5.31 (d, J=11.0 Hz, 1H), 3.83 (d, J=5.2 Hz, 2H), 3.70 (d, J=5.3 Hz, 2H), 3.60 (d, J=5.3 Hz, 2H), 3.49 (d, J=5.3 Hz, 2H). LC-MS. Retention time of 2.14 to 2.17 minutes. See FIGS. 9, 10, 11, and 12.

### Example 11. Methods of Making Certain Compounds

The following compound was made as follows:

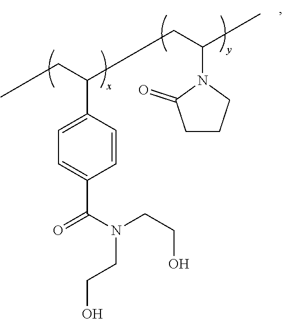

wherein the molar ratio of monomers x:y is 20:80 in the reaction solution prepared for polymerization.

376.45 mg (1.6 mmol) of N,N-Bis(2-hydroxyethyl)-4-vinylbenzamide (Ambeed Inc., Arlington Heights, IL) and 687.4 μL (6.4 mmol) of N-vinyl-2-pyrrolidone (Cat #26863, Chem-Impex International, Inc., Wood Dale, IL) was dissolved in 6 mL of benzyl alcohol (Cat #00371, Chem-Impex International, Inc., Wood Dale, IL). The solution was passed through 500 mg of SiliaBond Guanidine silica gel (Cat #R68230B, SiliCycle Inc., Quebec City, Canada) in a 10 mL syringe containing a 0.2 μm PTFE syringe filter to remove polymerization inhibitor. The filtered solution was added to a 10 mL glass vial containing 131.4 mg (0.8 mmol) of 2,2′-Azobis(2-methylpropionitrile) (Cat #755745, SigmaAldrich, St. Louis, MO) which is ˜10% molar equivalent of the total amount of monomers in the reaction solution. The vial was sealed with a PTFE coated rubber stopper and then purged with Argon gas for 15 minutes. The vial was placed into an aluminum heat block and the mixture was then heated to 95° C. for 2 hours. After cooling to room temperature, the solution was slowly pipetted into 80 mL of diethyl ether (Cat #309966, SigmaAldrich, St. Louis, MO). The polymerized product separated as a white powder and was pelleted via centrifugation. The diethyl ether was decanted, and the product was redissolved in a minimal amount of methanol (Cat #34860, SigmaAldrich, St. Louis, MO). The dissolved solution was slowly pipetted into another 80 mL volume of diethyl ether to precipitate the polymerized product and was again pelleted via centrifugation. The diethyl ether was decanted, and the product was dried at room temperature under vacuum for 24 hours. The resulting white powder, entitled poly(N,N-Bis(2-hydroxyethyl)-4-vinylbenzamide-co-N-vinyl-2-pyrrolidone) (x:y=20:80), was used without any further purification.

### Example 12. Methods of Making Certain Compounds

The following compound was made as follows:

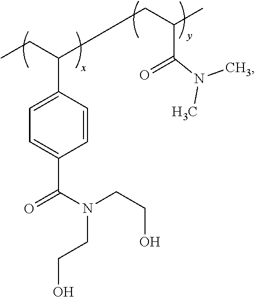

wherein the molar ratio of monomers x:y is 65:35 in the reaction solution prepared for polymerization.

305.9 mg (1.3 mmol) of N,N-Bis(2-hydroxyethyl)-4-vinylbenzamide (Ambeed Inc., Arlington Heights, IL) and 72.1 μL (0.7 mmol) of N,N-Dimethylacrylamide (Cat #D1091, TCI Chemicals, Portland, OR) was dissolved in 3 mL of benzyl alcohol (Cat #00371, Chem-Impex International, Inc., Wood Dale, IL). The solution was passed through 200 mg of SiliaBond Guanidine silica gel (Cat #R68230B, SiliCycle Inc., Quebec City, Canada) in a 10 mL syringe containing a 0.2 m PTFE syringe filter to remove polymerization inhibitor. The filtered solution was added to a 10 mL glass vial containing 32.8 mg (0.2 mmol) of 2,2′-Azobis(2-methylpropionitrile) (Cat #755745, SigmaAldrich, St. Louis, MO) which is ˜10% molar equivalent of the total amount of monomers in the reaction solution. The vial was sealed with a PTFE coated rubber stopper and then purged with Argon gas for 15 minutes. The vial was placed into an aluminum heat block and the mixture was then heated to 95° C. for 2 hours. After cooling to room temperature, the solution was slowly pipetted into 40 mL of diethyl ether (Cat #309966, SigmaAldrich, St. Louis, MO). The polymerized product separated as a white powder and was pelleted via centrifugation. The diethyl ether was decanted, and the product was redissolved in a minimal amount of methanol (Cat #34860, SigmaAldrich, St. Louis, MO). The dissolved solution was slowly pipetted into another 40 mL volume of diethyl ether to precipitate the polymerized product and was again pelleted via centrifugation. The diethyl ether was decanted, and the product was dried at room temperature under vacuum for 24 hours. The resulting white powder, entitled poly(N,N-Bis(2-hydroxyethyl)-4-vinylbenzamide-co-N,N-Dimethylacrylamide) (x:y=65:35), was used without any further purification.

### Example 13. Methods of Making Certain Compounds—Prophetic Example

The following compound could be made as follows:

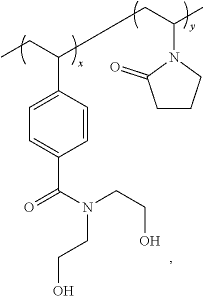

wherein the molar ratio of monomers x:y, in the reaction solution prepared for polymerization, may be varied from 100:0, of which results in an aromatic homopolymer after polymerization, to a ratio between 99:1 and 1:99, of which results in a copolymer after polymerization. The ratio of x:y is controlled by controlling the ratio of monomers in the reaction solution when the polymerization is initiated by AlBN. The nature of monomer addition to a growing chain is described in, for example, Nichifor, et al., Copolymers of N-alkylacrylamides and styrene as new thermosensitive materials, Polymer. Vol. 44, No. 10, pp. 3053-3060, 2003, for styrene-co-acrylamides (random addition), and Vijaykumar, et al., Copolymerization of N-Vinyl Pyrrolidone with Functionalized Vinyl Monomers: Synthesis, Characterization and Reactivity Relationships, Macromolecular Research. Vol. 17, No. 12, pp. 1003-1009, 2009, styrene-co-n-vinylpyrrolidones (gradient addition of styrene before n-vp).

A prescribed amount of N,N-Bis(2-hydroxyethyl)-4-vinylbenzamide (Ambeed Inc., Arlington Heights, IL) would be weighed out and combined with a prescribed amount of N-vinyl-2-pyrrolidone (Cat #26863, Chem-Impex International, Inc., Wood Dale, IL) such that the total amount of monomers would be equal to 8 millimoles. The monomers would then dissolve in 6 mL of benzyl alcohol (Cat #00371, Chem-Impex International, Inc., Wood Dale, IL). The solution would be passed through 500 mg of SiliaBond Guanidine silica gel (Cat #R68230B, SiliCycle Inc., Quebec City, Canada) in a 10 mL syringe containing a 0.2 μm PTFE syringe filter to remove polymerization inhibitor. The filtered solution would be added to a 10 mL glass vial containing 131.4 mg (0.8 mmol) of 2,2′-Azobis(2-methylpropionitrile) (Cat #755745, SigmaAldrich, St. Louis, MO) which would be ˜10% molar equivalent of the total amount of monomers in the reaction solution. The vial would be then sealed with a PTFE coated rubber stopper and purged with Argon gas for 15 minutes. The vial would be placed into an aluminum heat block and heated to 95° C. for 2 hours. After cooling to room temperature, the solution would be slowly pipetted into 80 mL of diethyl ether (Cat #309966, SigmaAldrich, St. Louis, MO). The polymerized product would separate as a white powder and would be pelleted via centrifugation. The diethyl ether would be decanted, and the product would be redissolved in a minimal amount of methanol (Cat #34860, SigmaAldrich, St. Louis, MO). The dissolved solution would be slowly pipetted into another 80 mL volume of diethyl ether to precipitate the polymerized product and again pelleted via centrifugation. The diethyl ether would be decanted, and the product would be then dried at room temperature under vacuum for 24 hours. The resulting white powder could be used without any further purification.

### Example 14. Methods of Making Certain Compounds—Prophetic Example

The following compound can be made as follows:

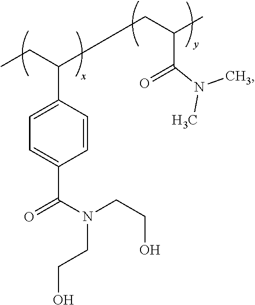

wherein the molar ratio of monomers x:y, in the reaction solution prepared for polymerization, may be varied from 100:0, of which results in an aromatic homopolymer after polymerization, to a ratio between 99:1 and 1:99, of which results in a copolymer after polymerization. The ratio of x:y is controlled by controlling the ratio of monomers in the reaction solution when the polymerization is initiated by AIBN. The nature of monomer addition to a growing chain is described in, for example, Nichifor, et al., Copolymers of N-alkylacrylamides and styrene as new thermosensitive materials, Polymer. Vol. 44, No. 10, pp. 3053-3060, 2003, for styrene-co-acrylamides (random addition), and Vijaykumar, et al., Copolymerization of N-Vinyl Pyrrolidone with Functionalized Vinyl Monomers: Synthesis, Characterization and Reactivity Relationships, Macromolecular Research. Vol. 17, No. 12, pp. 1003-1009, 2009, for styrene-co-n-vinylpyrrolidones (gradient addition of styrene before n-vp).

A prescribed amount of N,N-Bis(2-hydroxyethyl)-4-vinylbenzamide (Ambeed Inc., Arlington Heights, TL) would be weighed out and combined with a prescribed amount of N,N-Dimethylacrylamide (Cat #D1091, TCI Chemicals, Portland, OR) such that the total amount of monomers would be equal to 8 millimoles. The monomers would then dissolve in 6 mL of benzyl alcohol (Cat #00371, Chem-Impex International, Inc., Wood Dale, IL). The solution would be passed through 500 mg of SiliaBond Guanidine silica gel (Cat #R68230B, SiliCycle Inc., Quebec City, Canada) in a 10 mL syringe containing a 0.2 μm PTFE syringe filter to remove polymerization inhibitor. The filtered solution would be added to a 10 mL glass vial containing 131.4 mg (0.8 mmol) of 2,2′-Azobis(2-methylpropionitrile) (Cat #755745, SigmaAldrich, St. Louis, MO) which would be ˜10% molar equivalent of the total amount of monomers in the reaction solution. The vial would be then sealed with a PTFE coated rubber stopper and purged with Argon gas for 15 minutes. The vial would be placed into an aluminum heat block and heated to 95° C. for 2 hours. After cooling to room temperature, the solution would be slowly pipetted into 80 mL of diethyl ether (Cat #309966, SigmaAldrich, St. Louis, MO). The polymerized product would separate as a white powder and would be pelleted via centrifugation. The diethyl ether would be decanted, and the product would be redissolved in a minimal amount of methanol (Cat #34860, SigmaAldrich, St. Louis, MO). The dissolved solution would be slowly pipetted into another 80 mL volume of diethyl ether to precipitate the polymerized product and again pelleted via centrifugation. The diethyl ether would be decanted, and the product would be then dried at room temperature under vacuum for 24 hours. The resulting white powder can be used without any further purification.

### Example 15. Dye Aggregation Preventability and Reversibility

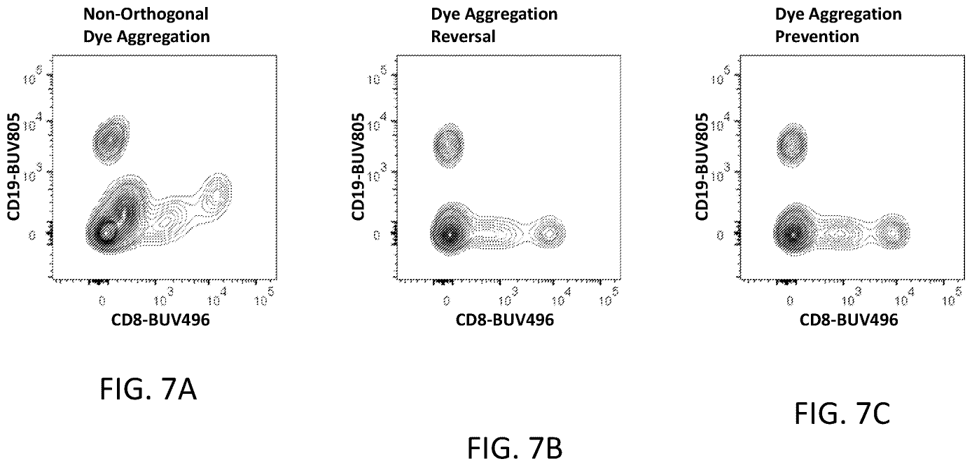

This Example demonstrates that the compound prepared in Example 11, poly(N,N-Bis(2-hydroxyethyl)-4-vinylbenzamide-co-N-vinyl-2-pyrrolidone) (x:y=20:80), is capable of preventing aggregation of labeled specific binding members, as well as being capable of reversing aggregation of labeled specific binding members. In this Example, the labeled specific binding members comprise the polymer dye conjugates CD8-BUV496 (Cat #612942, BD Biosciences, Franklin Lakes, NJ) and CD19-BUV805 (Cat #742007, BD Biosciences, Franklin Lakes, NJ). In a first experiment, a cocktail of the two polymer dye conjugates was prepared and diluted into PBS lacking any dispersants of the present disclosure. The PBS solution containing the cocktail of polymer dye conjugates was allowed to sit at room temperature for one hour, before the addition of fresh whole blood anticoagulated with EDTA, to label CD8+ and CD19+ cells with the CD8-BUV496 and CD19-BUV805 polymer dye conjugates, respectively. Following the labeling procedure, the cells were subjected to flow cytometry analysis. As illustrated at FIG. 7A, when the labeling was conducted with polymer dye conjugates that had not been contacted with any dispersant of the present disclosure, clear aggregation of the polymer dye conjugates is observed.

The polymer dye conjugate aggregation seen in the experimental results shown at FIG. 7A is reversible. Specifically, in a second experiment, again a cocktail of the two polymer dye conjugates CD8-BUV496 and CD19-BUV805 was prepared and diluted into PBS lacking any dispersants of the present disclosure. The prepared solution containing the two polymer dye conjugates was allowed to sit at room temperature for one hour. Following the room temperature incubation period, a volume of 50 mg/mL poly(N,N-Bis(2-hydroxyethyl)-4-vinylbenzamide-co-N-vinyl-2-pyrrolidone) (x:y=20:80) in PBS was added to a final concentration of 5 mg/mL, mixed, and allowed to sit for 10 minutes. Subsequent to the addition of the dispersant, a sample of fresh whole blood anticoagulated with EDTA was added to label CD8+ and CD19+ cells with the CD8-BUV496 and CD19-BUV805 polymer dye conjugates, respectively. Following the labeling procedure, the cells were subjected to flow cytometry analysis. As illustrated at FIG. 7B, addition of the poly(N,N-Bis(2-hydroxyethyl)-4-vinylbenzamide-co-N-vinyl-2-pyrrolidone) (x:y=20:80) reversed polymer dye conjugate aggregation, leading to spectrally pure signatures corresponding to labeled CD8 and CD19 cell populations with the corresponding polymer dye conjugates. For reversal of aggregation, although not specifically illustrated time frames that the dispersant and polymer dye conjugates may be allowed to sit to achieve aggregation reversal comprise about 5-60 minutes, for example about 5-30 minutes, for example about 10-20 minutes.

The polymer dye conjugate aggregation seen in the experimental results of FIG. 7A is also preventable. Specifically, in a third experiment a volume of 50 mg/mL poly(N,N-Bis(2-hydroxyethyl)-4-vinylbenzamide-co-N-vinyl-2-pyrrolidone) (x:y=20:80) was added to a final concentration of 5 mg/mL in PBS. Next, the polymer dye conjugates CD8-BUV496 and CD19-BUV805 were added to the solution of PBS plus dispersant. The prepared sample was allowed to sit for one hour at room temperature, prior to the addition of fresh whole blood to label CD8+ and CD19+ cells with the CD8-BUV496 and CD19-BUV805 polymer dye conjugates, respectively. Following the labeling procedure, the cells were subjected to flow cytometry analysis. As illustrated at FIG. 7C, polymer dye conjugate aggregation was prevented in the presence of the dispersant, leading to spectrally pure signatures corresponding to labeled CD8 and CD19 cell populations with the corresponding polymer dye conjugates.

### Example 16. Dye Aggregation Preventability

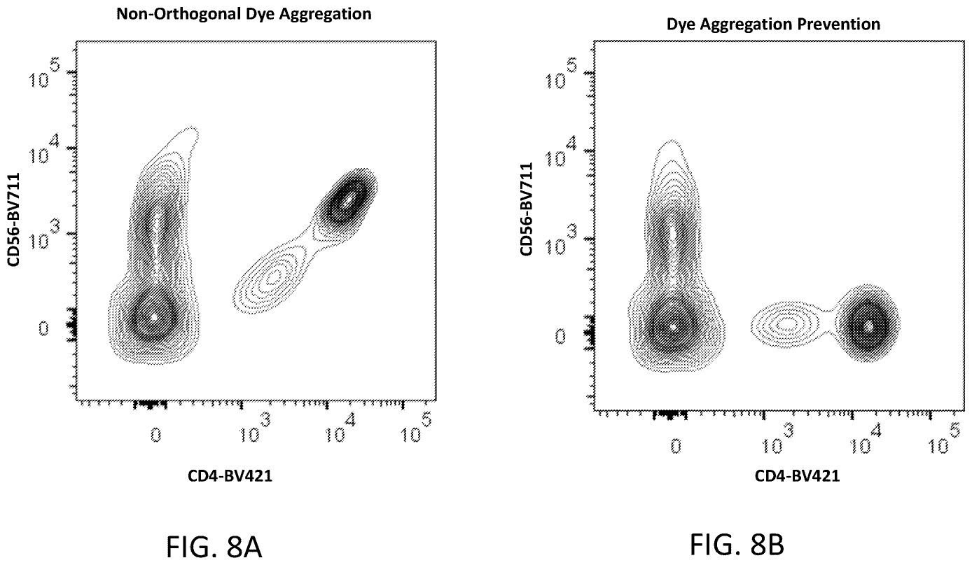

This Example demonstrates that the compound prepared in Example 12, poly(N,N-Bis(2-hydroxyethyl)-4-vinylbenzamide-co-N,N-Dimethylacrylamide) (x:y=65:35), is capable of preventing aggregation of labeled specific binding members. In this Example, the labeled specific binding members comprise the polymer dye conjugates CD4-BV421 (Cat #300531, BioLegend, San Diego, CA) and CD56-BV711 (Cat #362541, BioLegend, San Diego, CA). In a first experiment, a cocktail of the two polymer dye conjugates was prepared and diluted into PBS lacking any dispersants of the present disclosure. The PBS solution containing the cocktail of polymer dye conjugates was allowed to sit at room temperature for one hour, before the addition of fresh whole blood anticoagulated with EDTA, to label CD4+ and CD56+ cells with the CD4-BV421 and CD56-BV711 polymer dye conjugates, respectively. Following the labeling procedure, the cells were subjected to flow cytometry analysis. As illustrated at FIG. 8A, when the labeling was conducted with polymer dye conjugates that had not been contacted with any dispersant of the present disclosure, clear aggregation of the polymer dye conjugates is observed.

The polymer dye conjugate aggregation seen in the experimental results of FIG. 8A is preventable. Specifically, in a second experiment a volume of 50 mg/mL poly(N,N-Bis(2-hydroxyethyl)-4-vinylbenzamide-co-N,N-Dimethylacrylamide) (x:y=65:35) was added to a final concentration of 5 mg/mL in PBS. Next, the polymer dye conjugates CD4-BV421 and CD56-BV711 were added to the solution of PBS plus dispersant. The prepared sample was allowed to sit for one hour at room temperature, prior to the addition of fresh whole blood to label CD4+ and CD56+ cells with the CD4-BV421 and CD56-BV711 polymer dye conjugates, respectively. Following the labeling procedure, the cells were subjected to flow cytometry analysis. As illustrated at FIG. 8B, polymer dye conjugate aggregation was prevented in the presence of the dispersant, leading to spectrally pure signatures corresponding to labeled CD4 and CD56 cell populations with the corresponding polymer dye conjugates.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

1. A compound having the following structure: 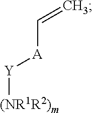
wherein:
A is a six-membered aryl or a six-membered heteroaryl, wherein heteroaryl comprises one to three heteroatoms selected from the group consisting of N, O, S, and combinations thereof;
Y is, individually in each instance, C═O or SO₂;
wherein R¹ and R² are each, individually in each instance, C₁₋₆alkyl or [PEG]ₚ—R^{A};
R^{A} is H or C₁₋₆alkyl;
subscript m is an integer of 1 to 3; and
subscript p is an integer of 1 to 10.

2. The compound of claim 1, having the following structure: 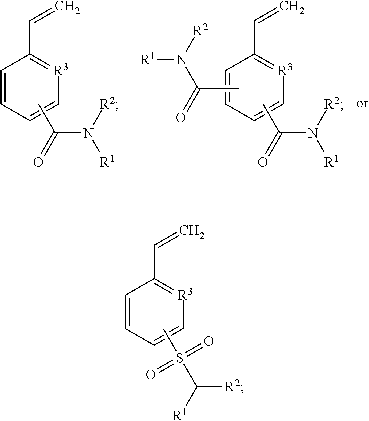
wherein R¹ and R² are each, individually in each instance, methyl or —C₂H₂O—[C₂H₂O]ₙ—R⁴;
R³ is CH or N;
R⁴ is H or methyl; and
wherein subscript n is 0, 1, 2, 3, 4, or 5.

3.-4. (canceled)

5. The compound of claim 1, wherein the compound has the following structure:

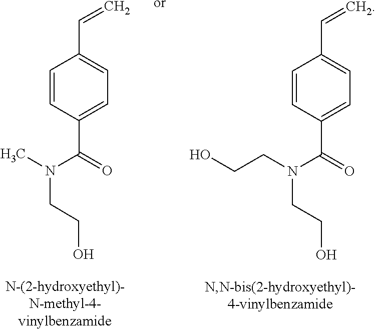 [SMILES: C=Cc1ccc(C(=O)N(C)CCO)cc1 ; "or" ; C=Cc1ccc(C(=O)N(CCO)CCO)cc1]

6. A polymer made by polymerizing a solution or mixture comprising at least one compound of claim 1.

7. A polymer comprising a saturated hydrocarbon substituted with at least one or more substituents,
wherein each substituent, individually in each instance is:
(a) selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; heteroaryl-heteroalkyl; and combinations thereof; and
(b) non-ionic at 6≤pH≤8;
(c) optionally substituted with 1 to 6 substituents selected from the group consisting of polyethylene glycol (PEG), —OH, —SH, —NO₂, —NO₃, —C(O)OH, —NH₃, —CH₃, —CH₂—OH, —CH₂—CH₂—OH, ═O, and —CN, wherein the optional substituents are not further substituted;
wherein the polymer:
(c) is water soluble; and
(d) is transparent to 350 nm to 800 nm wavelength light.

8.-11. (canceled)

12. The polymer of claim 7, wherein the polymer comprises a structure selected from Formula (I): 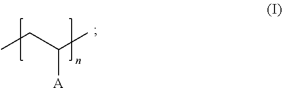
wherein
A is, independently in each instance, selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; and heteroaryl-heteroalkyl; and
subscript n is an integer from 1 to 100,000.

13. The polymer of claim 12, wherein A is selected from: 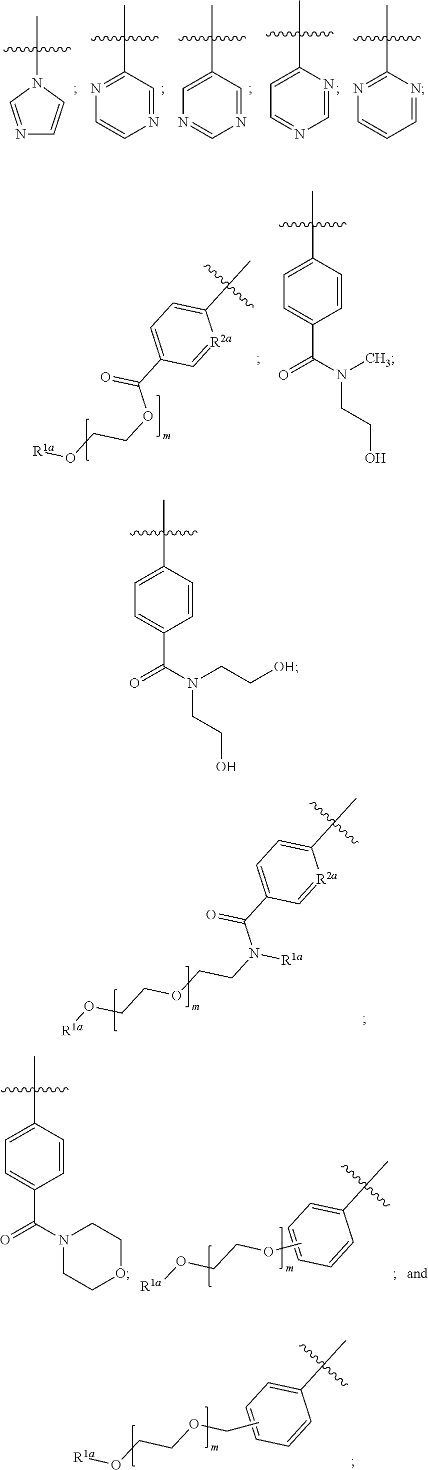
wherein
subscript m is an integer from 1 to 10;
R^{1a} is H, CH₃, or —C₂H₂O—[C₂H₂O]ₙ—R^{4a};
R^{2a} is CH or N;
R^{4a} is H or methyl; and
wherein subscript n is 0, 1, 2, 3, 4, or 5; and 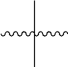
is the bond from A to Formula (I).

14.-15. (canceled)

16. The polymer of claim 7, wherein the polymer comprises a structure selected from Formula (IIa): 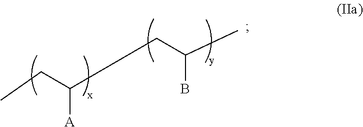
wherein
A and B are each, independently in each instance, selected from the group consisting of aryl; alkyl-aryl; aryl-alkyl; heteroalkyl-aryl; aryl-heteroalkyl; heteroaryl; alkyl-heteroaryl; heteroaryl-alkyl; heteroalkyl-heteroaryl; heteroaryl-heteroalkyl; cycloalkyl; heteroalkyl; and heterocycloalkyl; and combinations thereof;
wherein A is optionally substituted with one to three —NR¹R²;
wherein heteroalkyl and heterocycloalkyl are optionally substituted with 1 to 6 substituents selected from the group consisting of polyethylene glycol (PEG), —OH, —SH, —NO₂, —NO₃, —C(O)OH, —NH₃, —CH₃, —CH₂—OH, —CH₂—CH₂—OH, ═O, and —CN, wherein the optional substituents are not further substituted;
wherein R¹ and R² are each, individually in each instance, C₁₋₆alkyl or [PEG]ᵣ—R⁴;
R⁴ is H or C₁₋₆alkyl; and
subscript r is 1 to 10;
and the ratio of subscript x to subscript y is [0.05 to 0.25]:[0.75 to 0.95].

17. The polymer of claim 16, wherein the ratio of subscript x to subscript y is [0.15 to 0.2]:[0.8 to 0.85].

18. The polymer of claim 16, wherein B is selected from the group consisting of: 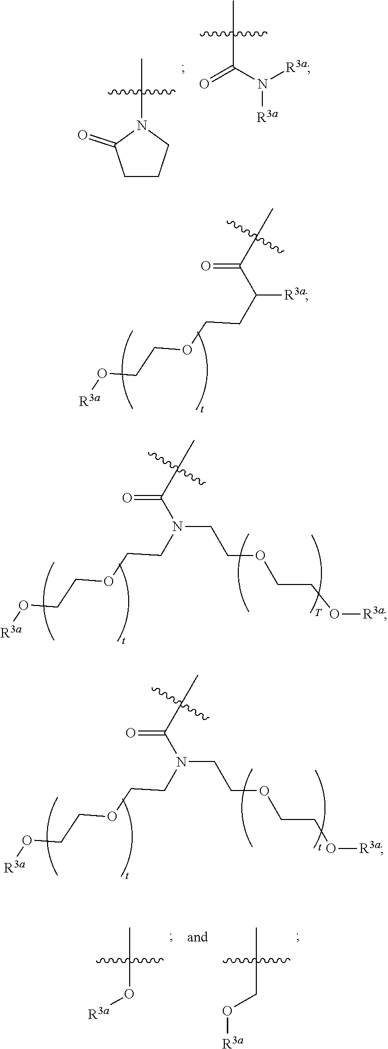
wherein subscript t is an integer from 0 to 10;
wherein R^{3a} is H or CH₃; and 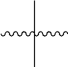
is the bond from B to Formula (II).

19. (canceled)

20. The polymer of claim 16, wherein A is selected from: 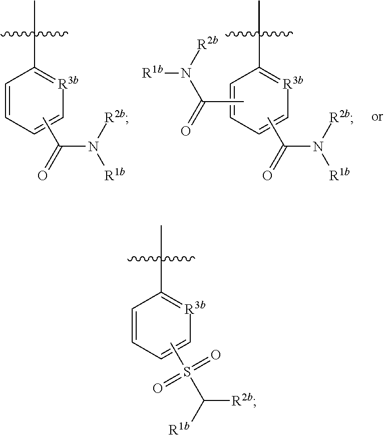
wherein R^{1b} and R^{2b} are each, individually in each instance, methyl or —C₂H₂O—[C₂H₂O]ₙ—R^{4b};
R^{3b} is CH or N;
R^{4b} is H or methyl; and
wherein subscript n is 0, 1, 2, 3, 4, or 5.

21. The polymer of claim 16, wherein A is selected from: 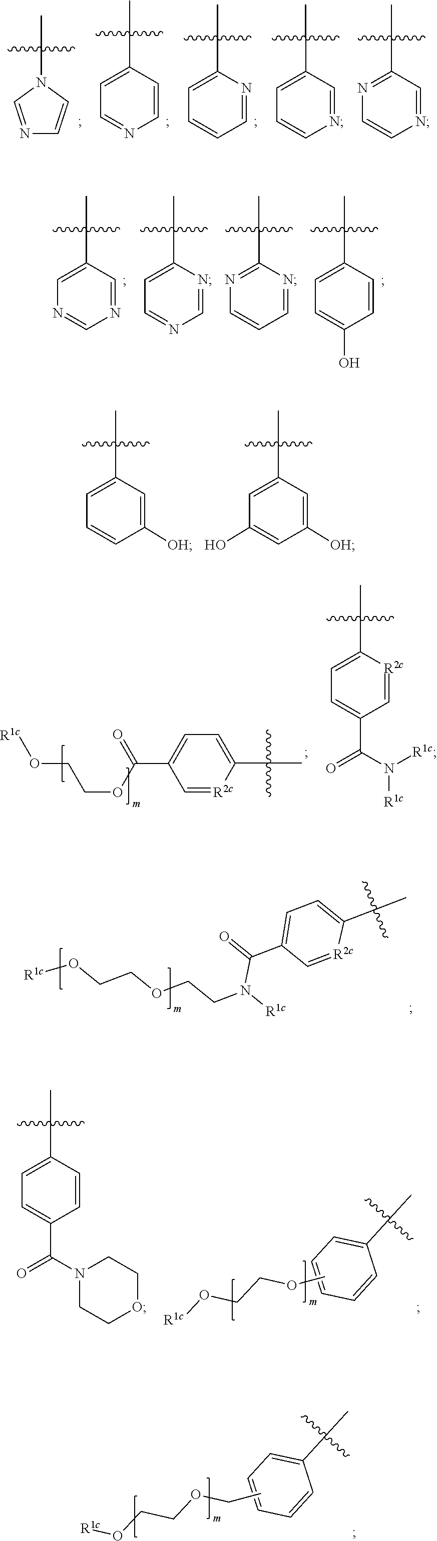
wherein
wherein subscript m is an integer from 1 to 10;
R^{1c} is H or CH₃;
R^{2c} is CH or N; and 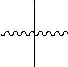
is the bond from A to Formula (IIa).

22. The polymer of claim 7, wherein the polymer comprises a structure selected from 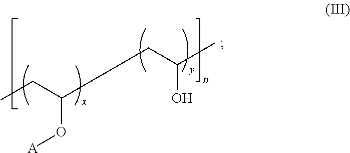
wherein
A is, independently in each instance, selected from the group consisting of aryl, alkyl-aryl;
heteroalkyl-aryl; heteroaryl, alkyl-heteroaryl, and heteroalkyl-heteroaryl;
subscript x is an integer from 1 to 50;
subscript y is an integer from 1 to 50; and
subscript n is an integer from 1 to 100,000.

23. The polymer of claim 7, wherein the polymer comprises the following structure: 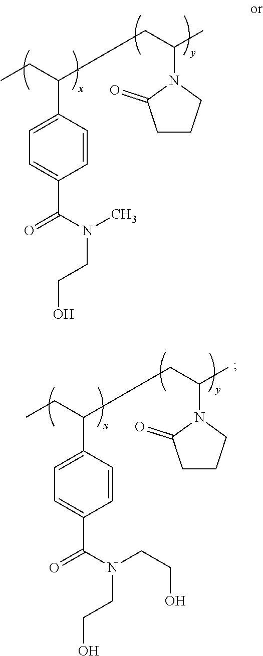
wherein the ratio of subscript x to subscript y is [0.05 to 0.25]:[0.75 to 0.95].

24.-30. (canceled)

31. The polymer of claim 7 wherein the polymer comprises a structure selected from Formula (IV) 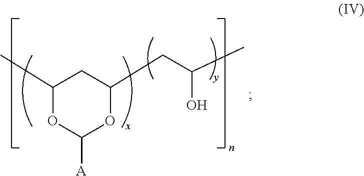
wherein
A is, independently in each instance, selected from the group consisting of aryl, alkyl-aryl; heteroalkyl-aryl; heteroaryl, alkyl-heteroaryl, and heteroalkyl-heteroaryl;
subscript x is an integer from 1 to 50;
subscript y is an integer from 1 to 50; and
subscript n is an integer from 1 to 100,000.

32. The polymer of claim 31, wherein the polymer comprises the following structure: 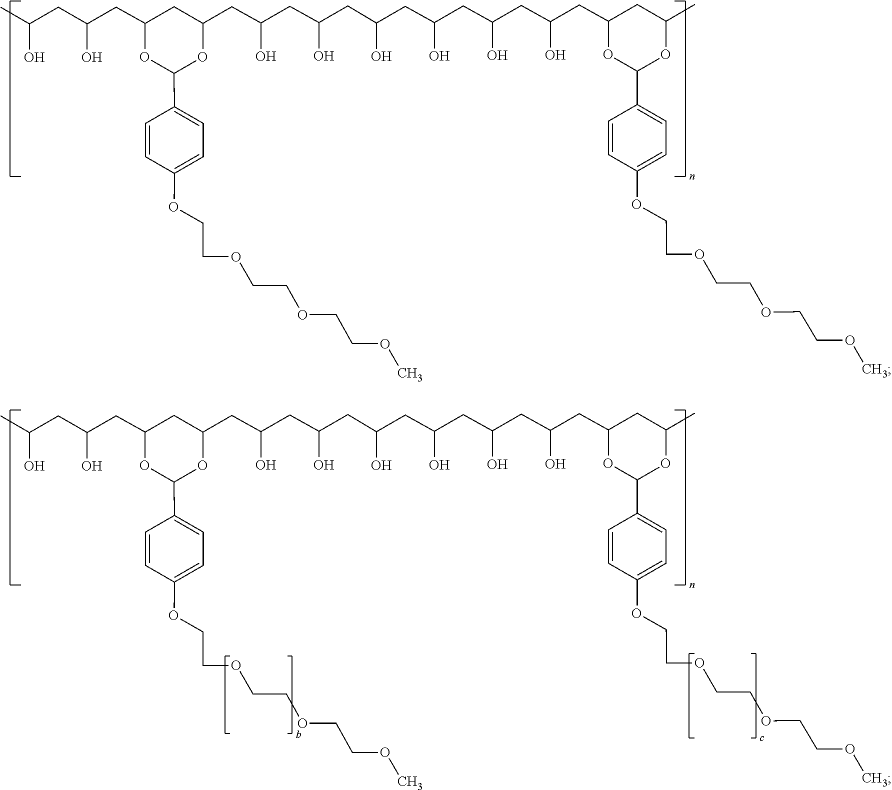 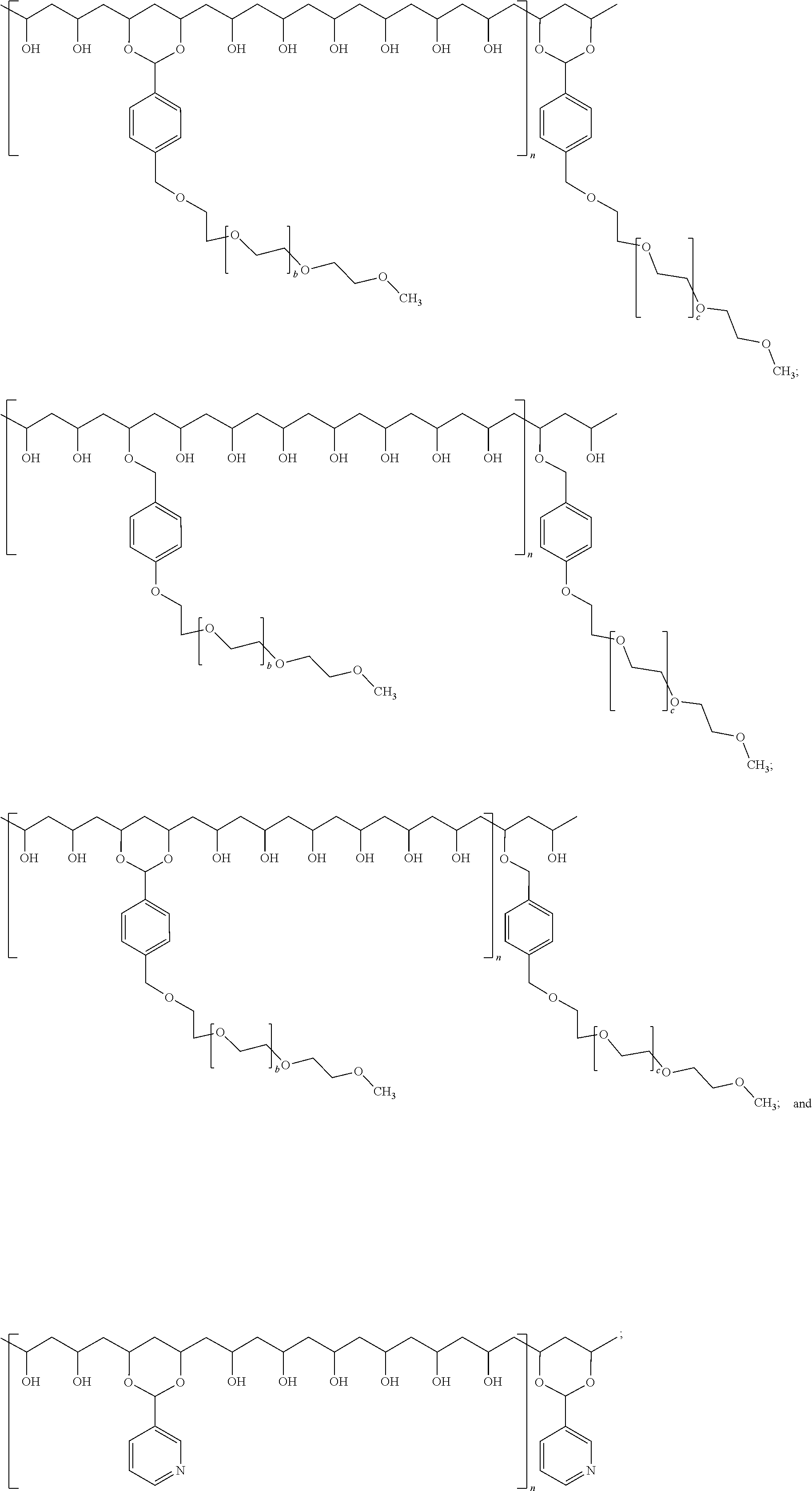
wherein subscript n is an integer from 1 to 100,000;
subscript b is an integer from 0 to 6; and
subscript c is an integer from 0 to 6.

33. (canceled)

34. A reaction mixture, comprising:
a dispersing agent and at least one fluorophore, wherein the dispersing agent comprises a polymer according to claim 7, or combinations thereof.

35. The reaction mixture according to claim 34, wherein the fluorophore comprises a polymeric dye, polymeric tandem dye, a tandem dye, or a combination thereof.

36. (canceled)

37. (canceled)

38. A method of labeling one or more of a target analyte present in a sample, comprising:
contacting the sample with the reaction mixture of claim 34 under conditions sufficient for specific binding of the target analyte to a labeled specific binding member included in the reaction mixture,
thereby providing a labeling composition contacted sample.

39. A method of preventing aggregation between two or more labeled specific binding members in a solution, comprising:
providing a solution of a dispersing agent comprising a polymer according to claim 7, or combinations thereof; and
separately adding each of the two or more labeled specific binding members to the solution to provide a labeled specific binding member-dispersing agent composition,
thereby preventing aggregation between the two or more labeled specific binding members.

40.-46. (canceled)
